(19) United States
(12) Patent Application Publication
(10) Pub. No.: US 2023/0149407 A1
(43) Pub. Date: May 18, 2023

(54) Methods of Treating Coronavirus Disease 2019

(71) Applicant: Pfizer Inc., New York, NY (US)

(72) Inventors: Michael Corbo, Flemington, NJ (US); Spencer Ian Danto, Sharon, MA (US); Tamas Andras Koncz, New York, NY (US); Elena Peeva, Tarrytown, NY (US); Hernan Valdez, V, New York, NY (US); Michael Steven Vincent, Newton, MA (US); Cara Margaret May Williams, Beverly, MA (US)

(73) Assignee: Pfizer Inc., New York, NY (US)

(21) Appl. No.: 17/907,707

(22) PCT Filed: Apr. 1, 2021
(86) PCT No.: PCT/IB2021/052749

§ 371 Date: Sep. 29, 2022

Related U.S. Application Data
(60) Provisional application No. 63/164,616, filed on Mar. 23, 2021.
(60) Provisional application No. 63/162,600, filed on Mar. 18, 2021.
(60) Provisional application No. 63/018,828, filed on May 1, 2020.
(60) Provisional application No. 63/005,303, filed on Apr. 4, 2020.

(51) Int. Cl. A61K 31/519; A61K 31/4725; A61P 31/14; A61P 11/00

(52) U.S. Cl. CPC A61K 31/519; A61K 31/4725; A61P 11/00; A61P 31/14

(57) ABSTRACT

The present invention is related to the discovery of new methods for treating the pathologic inflammatory response associated with patients infected with SARS-CoV-2 comprising administering orally to the patient in need of such treatment a therapeutically effective amount of a JAK inhibitor, a JAK/TYK inhibitor, or an IRAK4 inhibitor, or a combination thereof.

### FIELD OF THE INVENTION

The present invention relates to the discovery of new methods for treating patients that are afflicted with coronavirus disease 2019 (COVID-19).

### BACKGROUND OF THE INVENTION

Coronavirus disease 2019 (COVID-19) is a viral disease caused by a novel coronavirus, severe acute respiratory syndrome coronavirus 2 (SARS-CoV-2), that can cause acute respiratory distress syndrome (ARDS). ARDS is an acute lung disease due to destruction of the alveolar epithelium (diffuse alveolar damage) that is a response to a variety of injurious stimuli including viral pathogens such as SARS-CoV-2. The destruction of the alveolar epithelial barrier leads to an exudation of interstitial fluid and inflammatory cells (neutrophils and macrophages) that ultimately compromises lung dynamics, ventilation, and oxygenation. Clinically, development of ARDS is characterized by bilateral pulmonary infiltrates, decreased pulmonary compliance, and progressive hypoxemia. The severity of COVID-19 can vary from asymptomatic illness to severe or fatal disease. Many patients may rapidly (within 1 - 2 weeks of infection) develop dyspnea and pneumonia and require hospitalization for respiratory support. Of these hospitalized patients, 20 - 30% have required admission to intensive care units (ICUs) for ventilatory support due to development of ARDS, with ventilatory failure being a major cause of overall mortality due to COVID-19.

The genome sequence of SARS-CoV-2 was sequenced from isolates from nine patients in Wuhan, China and found to be of the genus betacoronovirus sharing about 79% homology with severe acute respiratory syndrome coronavirus (SARS-CoV), the causative agent of the SARS outbreak in 2002-2003. Preclinical data from betacoronaviruses similar to SARS-CoV-2 suggest that the pathogenic characteristics of progressive disease are dominated by an intense inflammatory response. The ultimate result is progressive destruction of the alveolar epithelium leading to ARDS. Moreover, the exudative phase of ARDS is due, at least in part, to a pro-inflammatory response involving influx of innate immune cells (neutrophils and macrophages) and elevations of inflammatory cytokines such as interleukin (IL)-6, IL-8, and tumor necrosis factor (TNF)-α, with higher levels of both IL-6 and IL-8 levels being correlated with increased mortality. While innate immune signaling is likely important for the initial response to SARS-CoV-2 infection, once pneumonia has developed, immunomodulatory therapy may be beneficial in reducing the deleterious effects of lung inflammation and mitigating progressive lung injury.

Pre-clinical coronavirus challenge models demonstrate that Janus Kinase (JAK) dependent pathways play a role in driving pathologic inflammation. Elevated JAK-dependent pro-inflammatory cytokines such as interferon alpha (IFNα), interferon beta (IFNβ), interferon gamma (IFN-γ), interleukin-6 (IL-6), interleukin-12 (IL-12) and inflammatory signatures in human SARS-CoV-2 patients are indicative of broad immune activation. Inhibition of JAK-dependent pathways reduces signaling for multiple pathogenic pro-inflammatory cytokines responsible for tissue damage including both innate and adaptive pathways that are present in SARS-CoV-2 infected patients. The JAK family of non-receptor protein tyrosine kinases consists of JAK1, JAK2, JAK3 and tyrosine kinase 2 (TYK2).

Interleukin-1 receptor associated kinase (IRAK) 4 is a serine, threonine kinase that plays a role in intracellular signaling node downstream of the mydossome associated Toll-Like receptor (TLR 1, 2, 4, 5, 6, 7, 8, 9 and 10) and the interleukin (IL)-1 family receptors (IL-1R, IL-18R and IL-33R). There is emerging evidence of increased activation of these pathways in patients who succumb to SARS-COVID-19 compared to survivors. Rare genetic variants in humans lacking IRAK4 or MYD88 are susceptible to infections with pyogenic bacteria, but there is no evidence of increased viral susceptibility, presumably due to redundant innate pathways recognizing viral nucleic acids upstream of the nuclear factor kappa-light-chain-enhancer of activated B cells (NF-κB). Furthermore, kinase inhibition preserves the IRAK4 scaffolding function within the myddosome and NF-κB activation downstream of TLR activation, suggesting a less immunosuppressive profile than the genetic deletion. In addition to inflammatory signals coming directly from the virus and anti-viral host response, another driver of inflammatory tone is the massive production of cellular debris presenting damage associated molecular patterns (DAMPs) that also signal through the TLR family of receptors.

There are currently no established treatments for COVID-19 infections and therefore a need exits for therapies that prevent the progression of infection to severe disease and death. The present invention provides methods for treating SARS-CoV-2 patients by inhibiting the inflammatory pathways activated by COVID-19 infection.

### SUMMARY OF THE INVENTION

The present invention provides a method of treating a patient infected with SARS-CoV-2 comprising administering to the patient in need of such treatment a therapeutically effective amount of a JAK inhibitor or a pharmaceutically acceptable salt thereof.

In another embodiment, the present invention provides a pharmaceutical composition comprising a therapeutically effective amount of a JAK inhibitor and at least one pharmaceutically acceptable vehicle, diluent or carrier.

In another embodiment, the present invention provides a method of treating a patient infected with SARS-CoV-2 comprising administering to the patient in need of such treatment a therapeutically effective amount of a tyrosine kinase 2 (TYK2) inhibitor or a pharmaceutically acceptable salt thereof.

In another embodiment, the present invention provides a pharmaceutical composition comprising a therapeutically effective amount of a TYK2 inhibitor and at least one pharmaceutically acceptable vehicle, diluent or carrier.

In another embodiment, the present invention provides a method of treating a patient infected with SARS-CoV-2 comprising administering to the patient in need of such treatment a therapeutically effective amount of an IRAK4 inhibitor or a pharmaceutically acceptable salt thereof.

In another embodiment, the present invention provides a pharmaceutical composition comprising a therapeutically effective amount of a IRAK4 inhibitor and at least one pharmaceutically acceptable vehicle, diluent or carrier.

In another embodiment, the present invention provides a method of treating a patient infected with SARS-CoV-2 comprising administering to the patient in need of such treatment a therapeutically effective amount of a pharmaceutical combination comprising at least two inhibitors selected from an IRAK4 inhibitor or a pharmaceutically acceptable salt thereof, a JAK inhibitor or a pharmaceutically acceptable salt thereof, and a TYK2 inhibitor or a pharmaceutically acceptable salt thereof, wherein the combination is administered simultaneously or sequentially.

In another embodiment, the present invention provides a pharmaceutical composition comprising a therapeutically effective amount of a pharmaceutical combination comprising at least one pharmaceutically acceptable vehicle, diluent or carrier and at least two inhibitors selected from an IRAK4 inhibitor or a pharmaceutically acceptable salt thereof, a JAK inhibitor or a pharmaceutically acceptable salt thereof, and a TYK2 inhibitor or a pharmaceutically acceptable salt thereof.

### BRIEF DESCRIPTION OF THE DRAWINGS

FIG. 1 provides a diagram of the JAK and JAK/TYK biological pathways and the cytokines/chemokines associated with these pathways.

FIG. 2 provides a diagram of 3-((3R,4R)-4-Methyl-3-(methyl(7H-pyrrolo[2,3-d]pyrimidin-4-yl)amino)piperidin-1-yl)-3-oxopropanenitrile’s (tofacitinib’s) mechanism of action.

FIG. 3 provides a diagram of tofacitinib’s effect on IFNα, IFNγ, TNFα, IL-2, IL4, IL-6, IL-7, IL-10, IL-12, IL13, IL-15, and IL-23 levels.

FIG. 4 provides a diagram of tofacitinib’s effect on IL-6, IFNα, IFNβ, and TNFα levels.

FIG. 5A provides a diagram of tofacitinib’s effect on neutrophil levels in humans following administration of tofacitinib over 12 months.

FIG. 5B provides a diagram of tofacitinib’s effect on total lymphocyte levels in humans following administration of tofacitinib over 12 months.

FIG. 6 provides a diagram of 1-(((2S,3S,4S)-3-Ethyl-4-fluoro-5-oxopyrrolidin-2-yl)methoxy)-7-methoxyisoquinoline-6- carboxamide’s (PF-06650833’s) effect on IL-6, IFNα, IFNγ, and TNFα levels in humans.

FIG. 7A provides a diagram representing the proposed 10 mg tofacitinib BID Phase 2 clinical study design for treating COVID-19 disease in patients.

FIG. 7B provides a diagram representing the proposed 400 mg PF-06650833 QD Phase 2 clinical study design for treating COVID-19 disease in patients.

FIG. 7C provides a diagram representing the proposed 200 mg PF-06650833 Q6H Phase 2 clinical study design for treating COVID-19 disease in patients.

FIG. 8 provides a diagram representing the proposed 400 mg PF-06650833 QD Phase 2 clinical study design for treating COVID-19 disease in patients up to 28 days.

FIG. 9 provides data demonstrating suppression of CRP in healthy adults following administration of PF-06650833.

FIG. 10 provides a schematic representation of the mechanism of action for IRAK4.

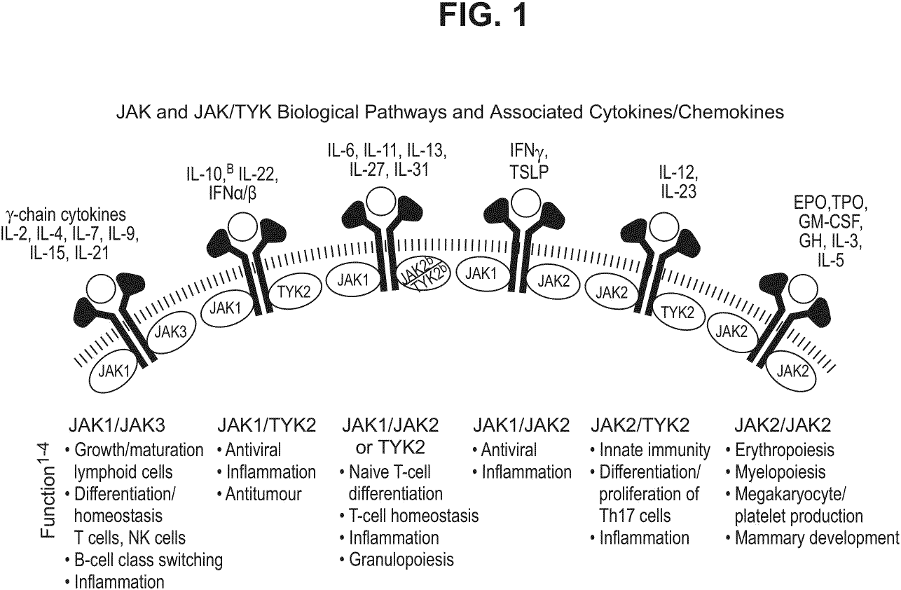

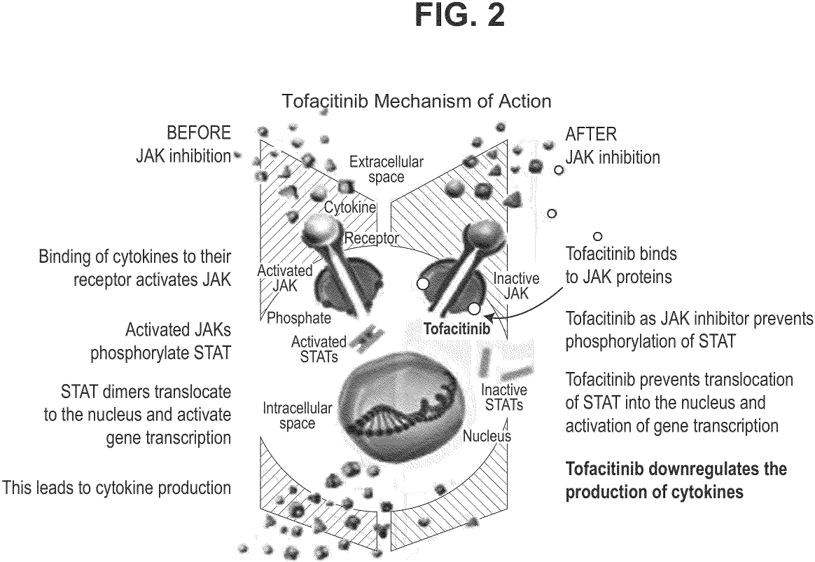

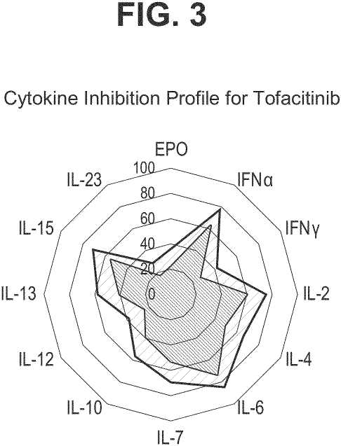

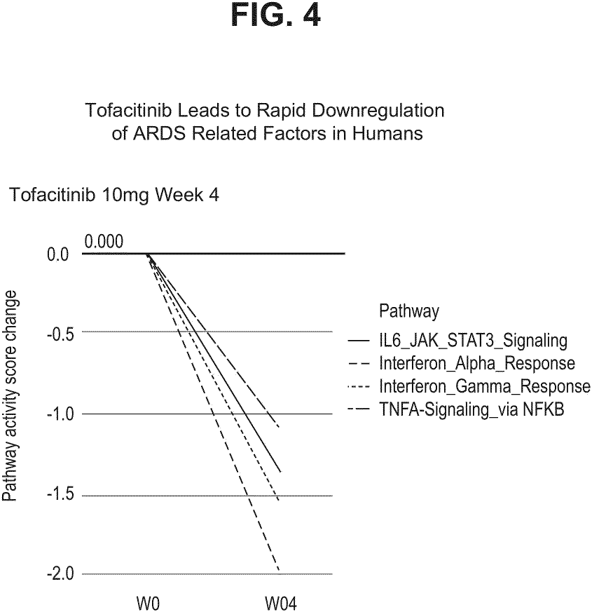

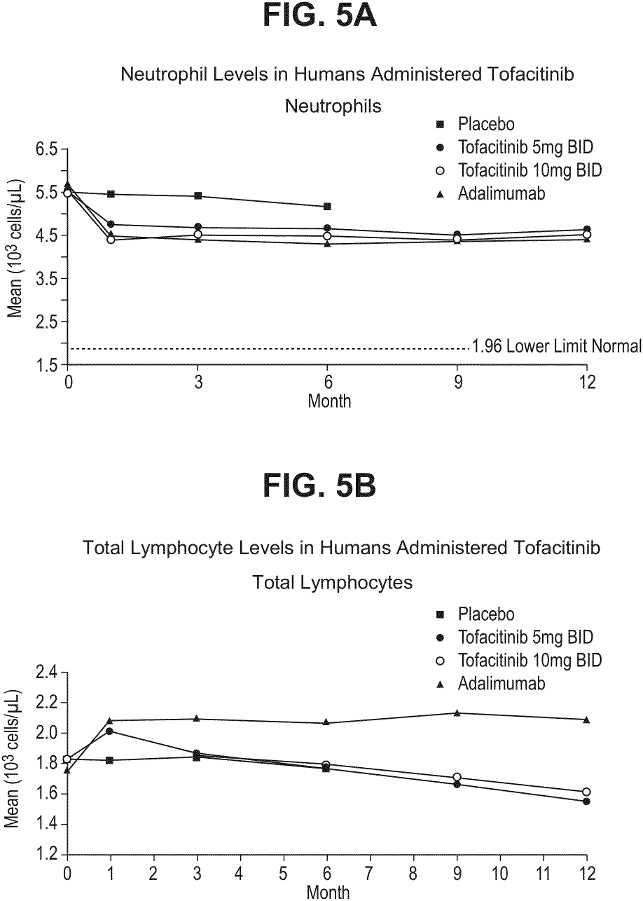

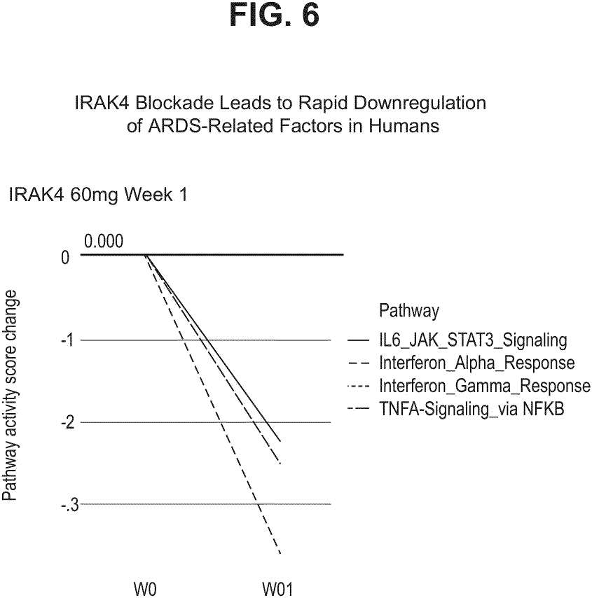

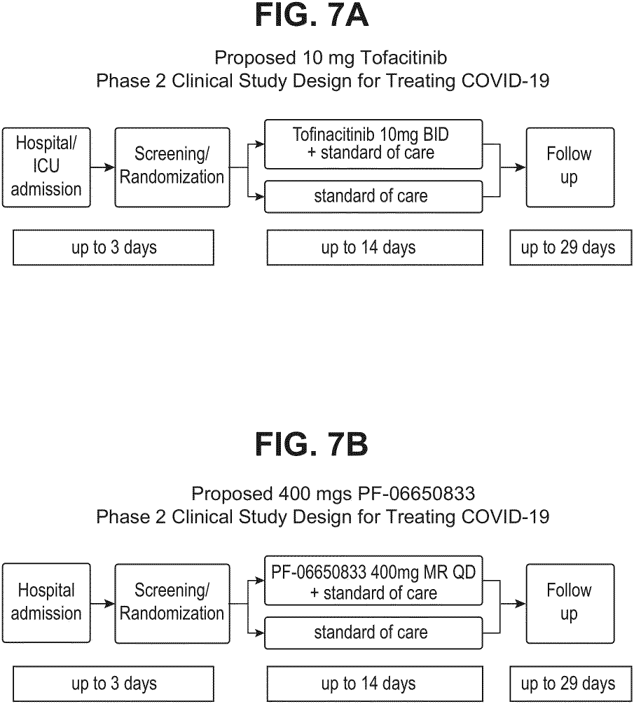

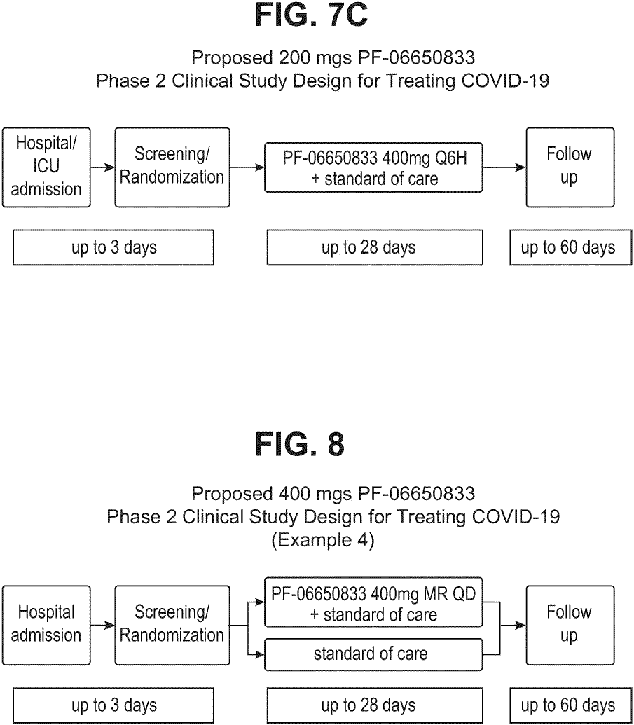

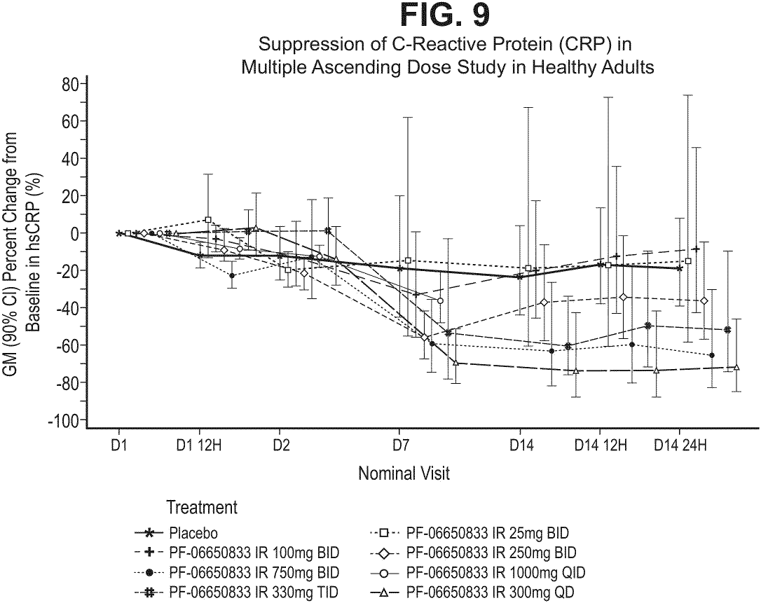

### DETAILED DESCRIPTION OF THE INVENTION

There are three clinical phases associated with patients infected with SARS-CoV-2 that utilize JAK and/or IRAK4 dependent pathways.

The first phase is characterized by robust virus replication that initiates the patient’s anti-viral defense that includes: early IFN response; inflammatory monocyte-macrophage and neutrophil infiltration; and pro-inflammatory cytokines and chemokines. An effective endogenous response at this stage leads to: minimal epithelial and endothelial cell apoptosis; reduced vascular leakage; optimal T cell and antibody responses; and effective virus clearance. Reducing JAK and/or IRAK4 dependent cytokines at this stage may not be desired.

The second phase is associated with high fever, hypoxemia, and progression to pneumonia-like symptoms despite a progressive decline in virus titers towards the end of this phase. Anti-viral cytokines and chemokines at this stage lead to an overexuberant response including monocyte/macrophage and polymorphonuclear leukocytes (neutrophils, eosinophils, and basophils). Reducing pathologic levels of JAK and/or IRAK4 dependent cytokines/chemokines may have benefit at this stage of disease.

The third phase, of which ~20% of patients progress, is characterized by ARDS and often results in death. Due to the progressive decline in virus titers, this phase may result from overexpression of pro-inflammatory cytokines/chemokines. Reducing pathologic levels of JAK and/or IRAK4 dependent cytokines/chemokines may have benefit at this stage of disease.

3-((3R,4R)-4-Methyl-3-(methyl(7H-pyrrolo[2,3-d]pyrimidin-4-yl)amino)piperidin-1-yl)-3-oxopropanenitrile (tofacitinib) is an orally bioavailable small molecule JAK inhibitor, approved for treating rheumatoid arthritis, psoriatic arthritis, and ulcerative colitis, that modulates the signaling of multiple JAK-dependent cytokine families. Tofacitinib binds to the adenosine triphosphate (ATP) binding site in the catalytic cleft of the kinase domain of JAK. As a result of binding to the ATP site, tofacitinib inhibits phosphorylation and activation of JAK, thereby preventing activation of gene transcription leading to decreased cytokine production. In particular, tofacitinib binds to the JAK family, JAK1 (IC₅₀=15 nM), JAK2 (IC₅₀=77 nM), JAK3 (IC₅₀=55 nM), and TYK2 (IC₅₀=488 nM) and reduces levels of IFNα, IFNβ, IFNγ, TNFα, IL-1, IL-2, IL4, IL-6, IL-7, IL-9, IL-10, IL-12, IL13, IL-15, IL-21, IL-23, and IL-27. Hodge JA, et al. Clin Exp Rheumatol. 2016;34(2):318-328.

The safety and clinical pharmacology of tofacitinib has been very well characterized. The pharmacodynamics (PD) of tofacitinib, in terms of cytokine inhibition and biomarkers of pharmacologic activity, have been characterized in in vitro studies, in animal models, and in vivo clinical studies. These data indicate rapid onset, followed by sustained pharmacologic activity over the dosing period. At a 10 mg BID dose, approximately 80% suppression of IL-6 may be expected, in addition to substantial inhibition of multiple other pro-inflammatory cytokines, such as IFNγ, IL-15, IL-21, and IL-27, supporting the use of tofacitinib 10 mg BID for prevention of overexpression of cytokines in COVID-19 infected patients. Given the high mortality in COVID-19 complicated by ARDS, and the overall case mortality rate in in COVID-19 patients, a tofacitinib dose of 10 mg immediate release formulation BID is expected to provide maximal cytokine suppression while maintaining an overall positive benefit-risk profile. Tofacitinib is to be administered to patients who have the ability to swallow two 5 mg tofacitinib IR tablets together or in sequence, i.e. patients not on a ventilator.

In another embodiment, the present invention provides a method of treating a patient infected with SARS-CoV-2 comprising administering to the patient in need of such treatment a therapeutically effective amount of tofacitinib or a pharmaceutically acceptable salt thereof.

In another embodiment, the present invention provides a method of treating a patient infected with SARS-CoV-2 comprising administering orally to the patient in need of such treatment 5 mgs of tofacitinib BID or an equivalent amount of tofacitinib in the form of a pharmaceutically acceptable salt thereof.

In another embodiment, the present invention provides a method of treating a patient infected with SARS-CoV-2 comprising administering orally to the patient in need of such treatment 10 mgs of tofacitinib BID or an equivalent amount of tofacitinib in the form of a pharmaceutically acceptable salt thereof.

In another embodiment, the present invention provides a method of treating a patient infected with SARS-CoV-2 comprising administering orally to the patient in need of such treatment 11 mgs of tofacitinib QD or an equivalent amount of tofacitinib in the form of a pharmaceutically acceptable salt thereof.

In another embodiment, the present invention provides a method of treating a patient infected with SARS-CoV-2 comprising administering orally to the patient in need of such treatment 5 mgs of tofacitinib BID or an equivalent amount of tofacitinib in the form of a pharmaceutically acceptable salt thereof for up to 14 days.

In another embodiment, the present invention provides a method of treating a patient infected with SARS-CoV-2 comprising administering orally to the patient in need of such treatment 10 mgs of tofacitinib BID or an equivalent amount of tofacitinib in the form of a pharmaceutically acceptable salt thereof for up to 14 days.

In another embodiment, the present invention provides a method of treating a patient infected with SARS-CoV-2 comprising administering orally to the patient in need of such treatment 11 mgs of tofacitinib BID or an equivalent amount of tofacitinib in the form of a pharmaceutically acceptable salt thereof for up to 14 days.

In another embodiment, the present invention provides a method of treating a patient infected with SARS-CoV-2 comprising administering orally to the patient in need of such treatment 11 mgs of tofacitinib QD or an equivalent amount of tofacitinib in the form of a pharmaceutically acceptable salt thereof for up to 14 days.

In another embodiment, the present invention provides a method of treating a patient infected with SARS-CoV-2 comprising administering orally to the patient in need of such treatment 10 mgs of tofacitinib QD or an equivalent amount of tofacitinib in the form of a pharmaceutically acceptable salt thereof for up to 14 days.

In another embodiment, the present invention provides a method of treating a patient infected with SARS-CoV-2 comprising administering orally to the patient in need of such treatment 5 mgs of tofacitinib QD or an equivalent amount of tofacitinib in the form of a pharmaceutically acceptable salt thereof for up to 14 days.

In another embodiment, the present invention provides a method of treating a patient infected with SARS-CoV-2 comprising administering orally to the patient in need of such treatment about 5 mgs of tofacitinib BID or an equivalent amount of tofacitinib in the form of a pharmaceutically acceptable salt thereof to about 11 mgs of tofacitinib BID or an equivalent amount of tofacitinib in the form of a pharmaceutically acceptable salt thereof for up to 14 days.

In another embodiment, the present invention provides a method of treating a patient infected with SARS-CoV-2 comprising administering orally to the patient in need of such treatment about 5 mgs of tofacitinib QD or an equivalent amount of tofacitinib in the form of a pharmaceutically acceptable salt thereof to about 11 mgs of tofacitinib QD or an equivalent amount of tofacitinib in the form of a pharmaceutically acceptable salt thereof for up to 14 days.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IL-6 levels are reduced by at least 20%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IL-6 levels are reduced by at least 20%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein IL-6 levels are reduced by at least 20%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein IL-6 levels are reduced by at least 20%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein IL-6 levels are reduced by at least 20%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IL-6 levels are reduced by at least 50%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IL-6 levels are reduced by at least 50%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein IL-6 levels are reduced by at least 50%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein IL-6 levels are reduced by at least 50%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein IL-6 levels are reduced by at least 50%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IL-6 levels are reduced by at least 75%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IL-6 levels are reduced by at least 75%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein IL-6 levels are reduced by at least 75%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein IL-6 levels are reduced by at least 75%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein IL-6 levels are reduced by at least 75%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IL-6 and IL-8 levels are each reduced by at least 20%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IL-6 and IL-8 levels are each reduced by at least 20%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein IL-6 and IL-8 levels are each reduced by at least 20%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein IL-6 and IL-8 levels are each reduced by at least 20%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein IL-6 and IL-8 levels are each reduced by at least 20%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IL-6, IL-8, and TNFα levels are each reduced by at least 20%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IL-6, IL-8, and TNFα levels are each reduced by at least 20%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein IL-6, IL-8, and TNFα levels are each reduced by at least 20%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein IL-6, IL-8, and TNFα levels are each reduced by at least 20%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein IL-6, IL-8, and TNFα levels are each reduced by at least 20%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IL-6, IFNα, IFNβ, and TNFα levels are each reduced by at least 20%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IL-6, IFNα, IFNβ, and TNFα levels are each reduced by at least 20%.

In another embodiment, the present invention provides a method of treating a patient infected with SARS-CoV-2 comprising administering orally to the patient in need of such treatment 5 mgs of tofacitinib BID, wherein IL-6, IFNα, IFNβ, and TNFα levels are each reduced by at least 20%.

In another embodiment, the present invention provides a method of treating a patient infected with SARS-CoV-2 comprising administering orally to the patient in need of such treatment 10 mgs of tofacitinib BID, wherein IL-6, IFNα, IFNβ, and TNFα levels are each reduced by at least 20%.

In another embodiment, the present invention provides a method of treating a patient infected with SARS-CoV-2 comprising administering orally to the patient in need of such treatment 11 mgs of tofacitinib QD, wherein IL-6, IFNα, IFNβ, and TNFα levels are each reduced by at least 20%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IL-6, IFNα, IFNβ, and TNFα levels are each reduced by at least 35%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IL-6, IFNα, IFNβ, and TNFα levels are each reduced by at least 35%.

In another embodiment, the present invention provides a method of treating a patient infected with SARS-CoV-2 comprising administering orally to the patient in need of such treatment 5 mgs of tofacitinib BID, wherein IL-6, IFNα, IFNβ, and TNFα levels are each reduced by at least 35%.

In another embodiment, the present invention provides a method of treating a patient infected with SARS-CoV-2 comprising administering orally to the patient in need of such treatment 10 mgs of tofacitinib BID, wherein IL-6, IFNα, IFNβ, and TNFα levels are each reduced by at least 35%.

In another embodiment, the present invention provides a method of treating a patient infected with SARS-CoV-2 comprising administering orally to the patient in need of such treatment 11 mgs of tofacitinib QD, wherein IL-6, IFNα, IFNβ, and TNFα levels are each reduced by at least 35%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IFNγ, IL-6, IL-15, IL-21, and IL-27 levels are each reduced by at least 20%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IFNγ, IL-6, IL-15, IL-21, and IL-27 levels are each reduced by at least 20%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein IFNγ, IL-6, IL-15, IL-21, and IL-27 levels are each reduced by at least 20%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein IFNγ, IL-6, IL-15, IL-21, and IL-27 levels are each reduced by at least 20%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein IFNγ, IL-6, IL-15, IL-21, and IL-27 levels are each reduced by at least 20%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IFNα, IFNγ, IL-2, IL4, IL-6, IL-7, IL-10, IL-12, IL13, IL-15, and IL-23 levels are each reduced by at least 20%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IFNα, IFNγ, IL-2, IL4, IL-6, IL-7, IL-10, IL-12, IL13, IL-15, and IL-23 levels are each reduced by at least 20%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein IFNα, IFNγ, IL-2, IL4, IL-6, IL-7, IL-10, IL-12, IL13, IL-15, and IL-23 levels are each reduced by at least 20%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein IFNα, IFNγ, IL-2, IL4, IL-6, IL-7, IL-10, IL-12, IL13, IL-15, and IL-23 levels are each reduced by at least 20%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein IFNα, IFNγ, IL-2, IL4, IL-6, IL-7, IL-10, IL-12, IL13, IL-15, and IL-23 levels are each reduced by at least 20%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein IFNα, IFNβ, IFNγ, TNFα, IL-1, IL-2, IL4, IL-6, IL-7, IL-9, IL-10, IL-12, IL13, IL-15, IL-21, IL-23, and IL-27 levels are each reduced by at least 10%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein IFNα, IFNβ, IFNγ, TNFα, IL-1, IL-2, IL4, IL-6, IL-7, IL-9, IL-10, IL-12, IL13, IL-15, IL-21, IL-23, and IL-27 levels are each reduced by at least 10%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein IFNα, IFNβ, IFNγ, TNFα, IL-1, IL-2, IL4, IL-6, IL-7, IL-9, IL-10, IL-12, IL13, IL-15, IL-21, IL-23, and IL-27 levels are each reduced by at least 10%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein IFNα, IFNβ, IFNγ, TNFα, IL-1, IL-2, IL4, IL-6, IL-7, IL-9, IL-10, IL-12, IL13, IL-15, IL-21, IL-23, and IL-27 levels are each reduced by at least 10%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein IFNα, IFNβ, IFNγ, TNFα, IL-1, IL-2, IL4, IL-6, IL-7, IL-9, IL-10, IL-12, IL13, IL-15, IL-21, IL-23, and IL-27 levels are each reduced by at least 10%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein total lymphocyte levels are reduced by at least 5%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein total lymphocyte levels are reduced by at least 5%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein total lymphocyte levels are reduced by at least 5%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein total lymphocyte levels are reduced by at least 5%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein total lymphocyte levels are reduced by at least 5%.

In another embodiment, the present invention provides a method of treating a patient infected with SARS-CoV-2 comprising administering to the patient in need of such treatment a therapeutically effective amount of tofacitinib, or a pharmaceutically acceptable salt thereof, wherein neutrophil levels are reduced by at least 15%.

In another embodiment, the present invention provides a method of treating a patient infected with SARS-CoV-2 comprising administering orally to the patient in need of such treatment 2.5-15 mgs of tofacitinib, or an equivalent amount of tofacitinib in the form of a pharmaceutically acceptable salt thereof, wherein neutrophil levels are reduced by at least 15%.

In another embodiment, the present invention provides a method of treating a patient infected with SARS-CoV-2 comprising administering orally to the patient in need of such treatment 5 mgs of tofacitinib BID, or an equivalent amount of tofacitinib in the form of a pharmaceutically acceptable salt thereof, wherein neutrophil levels are reduced by at least 15%.

In another embodiment, the present invention provides a method of treating a patient infected with SARS-CoV-2 comprising administering orally to the patient in need of such treatment 10 mgs of tofacitinib BID, or an equivalent amount of tofacitinib in the form of a pharmaceutically acceptable salt thereof, wherein neutrophil levels are reduced by at least 15%.

In another embodiment, the present invention provides a method of treating a patient infected with SARS-CoV-2 comprising administering orally to the patient in need of such treatment 11 mgs of tofacitinib QD, or an equivalent amount of tofacitinib in the form of a pharmaceutically acceptable salt thereof, wherein neutrophil levels are reduced by at least 15%.

It is to be understood that the method of treatment embodiments of the present invention can also be construed as use type embodiments. For example the embodiment: A method of treating a patient infected with SARS-CoV-2 comprising administering to the patient in need of such treatment a therapeutically effective amount of tofacitinib or a pharmaceutically acceptable salt thereof is understood to be equivalent to the use-type embodiment: Use of a therapeutically effective amount of tofacitinib or a pharmaceutically acceptable salt thereof for the treatment of SARS-CoV-2 infection.

IRAK4 kinase activity is required for inflammatory signaling mediated via action of the MYD88 associated toll like receptors (TLRs) plus the IL-1 family of receptors. Cytokines associated with TLR activity are increased in ARDS patients with a correlation in severity. Agents that inhibit IL-6 and IL-1 are currently being investigated for ARDS, IRAK4 inhibitors may have improved efficacy due to multi-cytokine impact (IL6, IL8, TNF, IL1, etc.). Inhibition of IRAK4 may reduce pathogen-associated molecular patterns (PAMPs) and damage-associated molecular patterns (DAMPs) that are increased during ARDS. Inhibition of IRAK4 should reduce PAMP/DAMP mediated immune cell activation, thereby decreasing cytokine, chemokine, MMP release (e.g. IL-6, IL-1, TNF(α) and downstream inflammatory processes (e.g. NETosis) that should curtail the cellular damage seen in ARDS. Humans with a genetic deletion of IRAK4 have impaired inflammatory signaling to a range of stimuli, but don’t appear more susceptible to viral illnesses. Therefore, small molecule inhibitors of IRAK4 represent a novel therapeutic strategy for modulation of innate immunity.

1-(((2S,3S,4S)-3-Ethyl-4-fluoro-5-oxopyrrolidin-2-yl)methoxy)-7-methoxyisoquinoline-6-carboxamide (PF-06650833) is an oral, selective small molecule reversible inhibitor of IRAK4 (IC₅₀ = 2.4 nM) that has demonstrated efficacy in a 12-week study in patients with moderately to severely active RA at doses of 200 and 400 mg as modified release (MR) tablets, and has demonstrated an acceptable safety profile in multiple human clinical trials. PF-06650833 dose-dependently inhibits blood transcriptional programs for type I interferons and IL-6 in healthy volunteers and RA patients. PF-06650833 also reduces levels of inflammatory monocytes and chemokine production from human bronchial epithelial cells, specifically IL-1β-stimulated release of CXCL8 and IL-6.

PF-06650833 has been generally well tolerated with an acceptable safety profile based on clinical studies conducted to date in healthy participants and patients with RA. Nonclinical efficacy and clinical biomarker data suggest that a continuous high degree of inhibition of IRAK4 is required for efficacy. An exploratory exposure response model suggests that maintaining PF-06650833 minimum observed concentration (Cₘᵢₙ) above the in vitro 90% inhibitory concentration ([IC₉₀]) is desirable for maximal C-reactive protein (CRP) reduction. A dose of 400 mg PF-06650833 modified release (MR) tablets administered QD to patients not on a ventilator or 200 mgs PF-06650833 immediate release (IR) suspension administered Q6H to patients on a ventilator is expected to inhibit TLR7/8 ligand-induced IL-6 production by approximately 90 and 95% at Cₘᵢₙ and Cₘₐₓ, respectively, which would translate to about 50-60% reduction in CRP. Given the high mortality in COVID-19 complicated by ARDS, and the overall case mortality rate in in COVID-19 patients, a PF-06650833 dose of 400 mg MR tablets QD, fasted, is expected to provide maximal cytokine suppression while maintaining an overall positive benefit-risk profile. Alternatively, a PF-06650833 dose of 200 mg IR suspension administered every six hours or four times a day (Q6H) would also be expected to suppress pro-inflammatory cytokines. It is to be understood that patients on a ventilator will receive PF-06650833 200 mg immediate release (IR) suspension administered orally (via NG or OG tube) every 6 hours, starting at the time of study enrollment for up to 28 days or to an earlier occurrence of one of the following: death, discharge from ICU, discharge from the hospital, or 14 days after return to their clinical baseline (as defined by need for supplementary oxygen) prior to SARS-CoV-2. Subjects who are able to take tablets by mouth (PO) will receive 400 mg PF-06650833 as modified release (MR) tablets orally once daily (QD), preferably under fasted conditions (at least 4 hours after and 1.5 hours before a meal). Patients on a ventilator may transition from the 200 mg IR suspension Q6H to the 400 mg MR tablets after they no longer require the ventilator and are able to swallow said tablets. Also, patients taking the 400 mg MR tablets may transition to the 200 mg IR suspension Q6H if they are unable to swallow the tablets or require a ventilator.

In another embodiment, the present invention provides a method of treating a patient infected with SARS-CoV-2 comprising administering to the patient in need of such treatment a therapeutically effective amount of PF-06650833 or a pharmaceutically acceptable salt thereof.

In another embodiment, the present invention provides a method of treating a patient infected with SARS-CoV-2 comprising administering orally to the patient in need of such treatment 400 mgs of PF-06650833 or an equivalent amount of PF-06650833 in the form of a pharmaceutically acceptable salt thereof.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein IL-6 levels are reduced by at least 20%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein IL-6 levels are reduced by at least 20%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein IL-6 levels are reduced by at least 20%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein IL-6 levels are reduced by at least 20%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein IL-6 levels are reduced by at least 50%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein IL-6 levels are reduced by at least 50%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein IL-6 levels are reduced by at least 50%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein IL-6 levels are reduced by at least 50%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein IL-6 levels are reduced by at least 75%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein IL-6 levels are reduced by at least 75%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein IL-6 levels are reduced by at least 75%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein IL-6 levels are reduced by at least 75%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein IL-6 levels are reduced by at least 90%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein IL-6 levels are reduced by at least 90%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein IL-6 levels are reduced by at least 90%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein IL-6 levels are reduced by at least 90%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein IL-6 and IL-1 levels are each reduced by at least 20%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein IL-6 and IL-1 levels are each reduced by at least 20%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein IL-6 and IL-1 levels are each reduced by at least 20%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein IL-6 and IL-1 levels are each reduced by at least 20%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein IL-6 IL-1, and TNFα levels are each reduced by at least 20%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein IL-6 IL-1, and TNFα levels are each reduced by at least 20%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein IL-6 IL-1, and TNFα levels are each reduced by at least 20%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein IL-6 IL-1, and TNFα levels are each reduced by at least 20%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein IL-6 IL-1, TNFα, IFNα, and IFNγ levels are each reduced by at least 20%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein IL-6 IL-1, TNFα, IFNα, and IFNγ levels are each reduced by at least 20%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein IL-6 IL-1, TNFα, IFNα, and IFNγ levels are each reduced by at least 20%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein IL-6 IL-1, TNFα, IFNα, and IFNγ levels are each reduced by at least 20%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein CRP levels are reduced by at least 20%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein CRP levels are reduced by at least 20%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein CRP levels are reduced by at least 20%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein CRP levels are reduced by at least 20%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein CRP levels are reduced by at least 50%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein CRP levels are reduced by at least 50%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein CRP levels are reduced by at least 50%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein CRP levels are reduced by at least 50%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein CRP levels are reduced by at least 20% and wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein CRP levels are reduced by at least 20% and wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein CRP levels are reduced by at least 20% and wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein CRP levels are reduced by at least 20% and wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein CRP levels are reduced by at least 50% and wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein CRP levels are reduced by at least 50% and wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein CRP levels are reduced by at least 50% and wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein CRP levels are reduced by at least 50% and wherein IL-6 IL-1, TNFα, IFNα, IFNγ, and CXCL8 levels are each reduced by at least 20%.

In another embodiment, the present invention provides a method of treating a patient infected with SARS-CoV-2 comprising administering to the patient in need of such treatment a therapeutically effective amount of PF-06650833, or a pharmaceutically acceptable salt thereof, wherein monocyte levels are reduced by at least 10%.

In another embodiment, the present invention provides a method of treating a patient infected with SARS-CoV-2 comprising administering to the patient in need of such treatment 100-800 mgs of PF-06650833, or an equivalent amount of PF-06650833 in the form of a pharmaceutically acceptable salt thereof, wherein monocyte levels are reduced by at least 10%.

In another embodiment, the present invention provides a method of treating a patient infected with SARS-CoV-2 comprising administering orally to the patient in need of such treatment 400 mgs of PF-06650833 QD, or an equivalent amount of PF-06650833 in the form of a pharmaceutically acceptable salt thereof, wherein monocyte levels are reduced by at least 10%.

In another embodiment, the present invention provides a method of treating a patient infected with SARS-CoV-2 comprising administering to the patient in need of such treatment 200 mgs of PF-06650833 Q6H, or an equivalent amount of PF-06650833 in the form of a pharmaceutically acceptable salt thereof, wherein monocyte levels are reduced by at least 10%.

1-((2S,5R)-5-((7H-Pyrrolo[2,3-d]pyrimidin-4-yl)amino)-2-methylpiperidin-1-yl)prop-2-en-1-one (PF-06651600) is a small molecule JAK3 inhibitor (IC₅₀ = 29 nM) currently being evaluated in clinical studies for inflammatory disorders.

In another embodiment, the present invention provides a method of treating a patient infected with SARS-CoV-2 comprising administering to the patient in need of such treatment a therapeutically effective amount of PF-06651600 or a pharmaceutically acceptable salt thereof.

In another embodiment, the present invention provides a method of treating a patient infected with SARS-CoV-2 comprising administering to the patient in need of such treatment a therapeutically effective amount of PF-06651600, or a pharmaceutically acceptable salt thereof, wherein IL-2 levels are reduced by at least 10%.

In another embodiment, the present invention provides a method of treating a patient infected with SARS-CoV-2 comprising administering orally to the patient in need of such treatment 50-200 mgs of PF-06651600, or an equivalent amount of PF-06651600 in the form of a pharmaceutically acceptable salt thereof, wherein IL-2 levels are reduced by at least 10%.

In another embodiment, the present invention provides a method of treating a patient infected with SARS-CoV-2 comprising administering orally to the patient in need of such treatment 100 mgs of PF-06651600 QD, or an equivalent amount of PF-06651600 in the form of a pharmaceutically acceptable salt thereof, wherein IL-2 levels are reduced by at least 10%.

In another embodiment, the present invention provides a method of treating a patient infected with SARS-CoV-2 comprising administering to the patient in need of such treatment a therapeutically effective amount of PF-06651600, or a pharmaceutically acceptable salt thereof, wherein IL-4 levels are reduced by at least 10%.

In another embodiment, the present invention provides a method of treating a patient infected with SARS-CoV-2 comprising administering orally to the patient in need of such treatment 50-200 mgs of PF-06651600, or an equivalent amount of PF-06651600 in the form of a pharmaceutically acceptable salt thereof, wherein IL-4 levels are reduced by at least 10%.

In another embodiment, the present invention provides a method of treating a patient infected with SARS-CoV-2 comprising administering orally to the patient in need of such treatment 100 mgs of PF-06651600 QD, or an equivalent amount of PF-06651600 in the form of a pharmaceutically acceptable salt thereof, wherein IL-4 levels are reduced by at least 10%.

In another embodiment, the present invention provides a method of treating a patient infected with SARS-CoV-2 comprising administering to the patient in need of such treatment a therapeutically effective amount of PF-06651600, or a pharmaceutically acceptable salt thereof, wherein IL-7 levels are reduced by at least 10%.

In another embodiment, the present invention provides a method of treating a patient infected with SARS-CoV-2 comprising administering orally to the patient in need of such treatment 50-200 mgs of PF-06651600, or an equivalent amount of PF-06651600 in the form of a pharmaceutically acceptable salt thereof, wherein IL-7 levels are reduced by at least 10%.

In another embodiment, the present invention provides a method of treating a patient infected with SARS-CoV-2 comprising administering orally to the patient in need of such treatment 100 mgs of PF-06651600 QD, or an equivalent amount of PF-06651600 in the form of a pharmaceutically acceptable salt thereof, wherein IL-7 levels are reduced by at least 10%.

In another embodiment, the present invention provides a method of treating a patient infected with SARS-CoV-2 comprising administering to the patient in need of such treatment a therapeutically effective amount of PF-06651600, or a pharmaceutically acceptable salt thereof, wherein IL-9 levels are reduced by at least 10%.

In another embodiment, the present invention provides a method of treating a patient infected with SARS-CoV-2 comprising administering orally to the patient in need of such treatment 50-200 mgs of PF-06651600, or an equivalent amount of PF-06651600 in the form of a pharmaceutically acceptable salt thereof, wherein IL-9 levels are reduced by at least 10%.

In another embodiment, the present invention provides a method of treating a patient infected with SARS-CoV-2 comprising administering orally to the patient in need of such treatment 100 mgs of PF-06651600 QD, or an equivalent amount of PF-06651600 in the form of a pharmaceutically acceptable salt thereof, wherein IL-9 levels are reduced by at least 10%.

In another embodiment, the present invention provides a method of treating a patient infected with SARS-CoV-2 comprising administering to the patient in need of such treatment a therapeutically effective amount of PF-06651600, or a pharmaceutically acceptable salt thereof, wherein IL-15 levels are reduced by at least 10%.

In another embodiment, the present invention provides a method of treating a patient infected with SARS-CoV-2 comprising administering orally to the patient in need of such treatment 50-200 mgs of PF-06651600, or an equivalent amount of PF-06651600 in the form of a pharmaceutically acceptable salt thereof, wherein IL-15 levels are reduced by at least 10%.

In another embodiment, the present invention provides a method of treating a patient infected with SARS-CoV-2 comprising administering orally to the patient in need of such treatment 100 mgs of PF-06651600 QD, or an equivalent amount of PF-06651600 in the form of a pharmaceutically acceptable salt thereof, wherein IL-15 levels are reduced by at least 10%.

In another embodiment, the present invention provides a method of treating a patient infected with SARS-CoV-2 comprising administering to the patient in need of such treatment a therapeutically effective amount of PF-06651600, or a pharmaceutically acceptable salt thereof, wherein IL-21 levels are reduced by at least 10%.

In another embodiment, the present invention provides a method of treating a patient infected with SARS-CoV-2 comprising administering orally to the patient in need of such treatment 50-200 mgs of PF-06651600, or an equivalent amount of PF-06651600 in the form of a pharmaceutically acceptable salt thereof, wherein IL-21 levels are reduced by at least 10%.

In another embodiment, the present invention provides a method of treating a patient infected with SARS-CoV-2 comprising administering orally to the patient in need of such treatment 100 mgs of PF-06651600 QD, or an equivalent amount of PF-06651600 in the form of a pharmaceutically acceptable salt thereof, wherein IL-21 levels are reduced by at least 10%.

In another embodiment, the present invention provides a method of treating a patient infected with SARS-CoV-2 comprising administering to the patient in need of such treatment a therapeutically effective amount of PF-06651600, or a pharmaceutically acceptable salt thereof, wherein IL-2, IL-4, IL-7, IL-9, IL-15 and IL-21 levels are each reduced by at least 10%.

In another embodiment, the present invention provides a method of treating a patient infected with SARS-CoV-2 comprising administering orally to the patient in need of such treatment 50-200 mgs of PF-06651600, or an equivalent amount of PF-06651600 in the form of a pharmaceutically acceptable salt thereof, wherein IL-2, IL-4, IL-7, IL-9, IL-15 and IL-21 levels are each reduced by at least 10%.

In another embodiment, the present invention provides a method of treating a patient infected with SARS-CoV-2 comprising administering orally to the patient in need of such treatment 100 mgs of PF-06651600 QD, or an equivalent amount of PF-06651600 in the form of a pharmaceutically acceptable salt thereof, wherein IL-2, IL-4, IL-7, IL-9, IL-15 and IL-21 levels are each reduced by at least 10%.

The present invention is related to methods for treating COVID-19 disease in a patient, the method comprising admistering to the patient in need of such treatment compounds which inhibit certain JAK, such as JAK1, JAK3, Tyk2/JAK1, and Tyk2. Accordingly, in another embodiment, the present invention provides a method of treating a patient infected with SARS-CoV-2 comprising administering to the patient in need of such treatment a therapeutically effective amount of a compound selected from the group consisting of:
[(1S)-2,2-difluorocyclopropyl]{(1R,5S)-3-[2-({5-fluoro-6-[(3S)-3-hydroxypyrrolidin-1-yl]pyridin-3-yl}amino)pyrimidin-4-yl]-3,8-diazabicyclo[3.2.1]oct-8-yl}methanone;
(1R,5S)-N-ethyl-3-[2-(1,2-thiazol-4-ylamino)pyrimidin-4-yl]-3,8-diazabicyclo[3.2.1]octane-8-carboxamide;
4-{(1R,5S)-8-[(2,2-difluorocyclopropyl)methyl]-3,8-diazabicyclo[3.2.1]oct-3-yl}-N-(1H-pyrazol-4-yl)pyrimidin-2-amine;
(1R,5S)-3-(2-{[5-chloro-6-(methylcarbamoyl)pyridin-3-yl]amino}pyrimidin-4-yl)-N-ethyl-3,8-diazabicyclo[3.2.1]octane-8-carboxamide;
cyclopropyl[(1R,5S)-3-(2-{[1-(2-hydroxyethyl)-1H-pyrazol-4-yl]amino}pyrimidin-4-yl)-3,8-diazabicyclo[3.2.1]oct-8-yl]methanone;
N-(1-methyl-1H-pyrazol-4-yl)-4-{(1R,5S)-8-[1-(methylsulfonyl)azetidin-3-yl]-3,8-diazabicyclo[3.2.1]oct-3-yl}pyrimidin-2-amine;
4-({4-[(1R,5S)-8-{[(1S)-2,2-difluorocyclopropyl]carbonyl}-3,8-diazabicyclo[3.2.1]oct-3-yl]pyrimidin-2-yl}amino)-N,6-dimethylpyridine-2-carboxamide;
5-({4-[(1R,5S)-8-{[(1R,2S)-2-fluorocyclopropyl]carbonyl}-3,8-diazabicyclo[3.2.1]oct-3-yl]pyrimidin-2-yl}amino)-N,3-dimethylpyridine-2-carboxamide;
cyclopropyl[(1R,5S)-3-{2-[(1-methyl-1H-pyrazol-4-yl)amino]pyrimidin-4-yl}-3,8-diazabicyclo[3.2.1]oct-8-yl]methanone;
3-{(1R,5S)-3-[2-(1H-pyrazol-4-ylamino)pyrimidin-4-yl]-3,8-diazabicyclo[3.2.1]oct-8-yl}butanenitrile;
5-({4-[(1R,5S)-8-(cyclopropylcarbonyl)-3,8-diazabicyclo[3.2.1]oct-3-yl]pyrimidin-2-yl}amino)-N-ethyl-3-methylpyridine-2-carboxamide;
3-[(1R,5S)-3-{2-[(1-methyl-1H-pyrazol-4-yl)amino]pyrimidin-4-yl}-3,8-diazabicyclo[3.2.1]oct-8-yl]butanenitrile;
5-({4-[(1R,5S)-8-(cyclopropylcarbonyl)-3,8-diazabicyclo[3.2.1]oct-3-yl]pyrimidin-2-yl}amino)-3-methylpyridine-2-carboxamide;
(1R,5S)-N-ethyl-3-(2-{[5-fluoro-6-(methylcarbamoyl)pyridin-3-yl]amino}pyrimidin-4-yl)-3,8-diazabicyclo[3.2.1]octane-8-carboxamide;
3-chloro-5-({4-[(1R,5S)-8-(cyclopropylcarbonyl)-3,8-diazabicyclo[3.2.1]oct-3-yl]pyrimidin-2-yl}amino)-N-methylpyridine-2-carboxamide;
(1R,5S)-3-{2-[(1-methyl-1H-pyrazol-4-yl)amino]pyrimidin-4-yl}-N-(propan-2-yl)-3,8-diazabicyclo[3.2.1]octane-8-carboxamide;
(3,3-difluorocyclobutyl)[(1R,5S)-3-{2-[(1-methyl-1H-pyrazol-4-yl)amino]pyrimidin-4-yl}-3,8-diazabicyclo[3.2.1]oct-8-yl]methanone;
1-({(1R,5S)-3-[2-(1H-pyrazol-4-ylamino)pyrimidin-4-yl]-3,8-diazabicyclo[3.2.1]oct-8-yl}methyl)cyclopropanecarbonitrile;
3-[(1R,5S)-3-{2-[(1-methyl-1H-pyrazol-4-yl)amino]pyrimidin-4-yl}-3,8-diazabicyclo[3.2.1]oct-8-yl]butanenitrile;
(1S,2R)-2-{[(1R,5S)-3-{2-[(1-methyl-1H-pyrazol-4-yl)amino]pyrimidin-4-yl}-3,8-diazabicyclo[3.2.1]oct-8-yl]carbonyl}cyclopropanecarbonitrile;
(1R,2S)-2-{[(1R,5S)-3-{2-[(1-methyl-1H-pyrazol-4-yl)amino]pyrimidin-4-yl}-3,8-diazabicyclo[3.2.1]oct-8-yl]carbonyl}cyclopropanecarbonitrile;
[(1R,2R)-2-fluorocyclopropyl][(1R,5S)-3-{2-[(1-methyl-1H-pyrazol-4-yl)amino]pyrimidin-4-yl}-3,8-diazabicyclo[3.2.1]oct-8-yl]methanone;
[(1R,2R)-2-fluorocyclopropyl][(1R,5S)-3-{2-[(1-methyl-1H-pyrazol-4-yl)amino]pyrimidin-4-yl}-3,8-diazabicyclo[3.2.1]oct-8-yl]methanone;
(1R,5S)-3-{2-[(1-methyl-1H-pyrazol-4-yl)amino]pyrimidin-4-yl}-3,8-diazabicyclo[3.2.1]octane-8-carboxamide;
(1R,5S)-3-{2-[(1-methyl-1H-pyrazol-4-yl)amino]pyrimidin-4-yl}-N-[5-(trifluoromethyl)pyridin-2-yl]-3,8-diazabicyclo[3.2.1]octane-8-carboxamide;
N,3-dimethyl-5-[(4-{(1R,5S)-8-[(3-methyloxetan-3-yl)methyl]-3,8-diazabicyclo[3.2.1]oct-3-yl}pyrimidin-2-yl)amino]pyridine-2-carboxamide;
{3-[(1R,5S)-3-{2-[(1-methyl-1H-pyrazol-4-yl)amino]pyrimidin-4-yl}-3,8-diazabicyclo[3.2.1]oct-8-yl]-1-(methylsulfonyl)azetidin-3-yl}acetonitrile;
4-({4-[8-(cyanoacetyl)-3,8-diazabicyclo[3.2.1]oct-3-yl]pyrimidin-2-yl}amino)-N-ethylbenzamide;
(1R,5S)-N-(cyanomethyl)-3-{2-[(1-methyl-1H-pyrazol-4-yl)amino]pyrimidin-4-yl}-3,8-diazabicyclo[3.2.1]octane-8-carboxamide;
5-({4-[(1R,5S)-8-{[(1S,2R)-2-fluorocyclopropyl]carbonyl}-3,8-diazabicyclo[3.2.1]oct-3-yl]pyrimidin-2-yl}amino)-N,3-dimethylpyridine-2-carboxamide;
5-({4-[(1R,5S)-8-(cis-3-cyanocyclobutyl)-3,8-diazabicyclo[3.2.1]oct-3-yl]pyrimidin-2-yl}amino)-N,3-dimethylpyridine-2-carboxamide;
5-({4-[(1R,5S)-8-{[(1R)-2,2-difluorocyclopropyl]methyl}-3,8-diazabicyclo[3.2.1]oct-3-yl]pyrimidin-2-yl}amino)-N,3-dimethylpyridine-2-carboxamide;
N,3-dimethyl-5-({4-[(1R,5S)-8-(1,2-oxazol-5-ylmethyl)-3,8-diazabicyclo[3.2.1]oct-3-yl]pyrimidin-2-yl}amino)pyridine-2-carboxamide;
2-[5-({4-[(1R,5S)-8-{[(1S)-2,2-difluorocyclopropyl]carbonyl}-3,8-diazabicyclo[3.2.1]oct-3-yl]pyrimidin-2-yl}amino)pyridin-2-yl]-2-methylpropanenitrile;
3-{(1R,5S)-3-[2-(1H-pyrazol-4-ylamino)pyrimidin-4-yl]-3,8-diazabicyclo[3.2.1]oct-8-yl}propanenitrile;
(1R,5S)-N-ethyl-3-[2-(1H-pyrazol-4-ylamino)pyrimidin-4-yl]-3,8-diazabicyclo[3.2.1]octane-8-carboxamide;
4-[(1R,5S)-8-{[(1S)-2,2-difluorocyclopropyl]methyl}-3,8-diazabicyclo[3.2.1]oct-3-yl]-N-(1-methyl-1H-pyrazol-4-yl)pyrimidin-2-amine;
[(1S)-2,2-difluorocyclopropyl][(1R,5S)-3-(2-{[5-fluoro-6-(2-hydroxyethyl)pyridin-3-yl]amino}pyrimidin-4-yl)-3,8-diazabicyclo[3.2.1]oct-8-yl]methanone;
[(1S)-2,2-difluorocyclopropyl][(1R,5S)-3-(2-{[5-fluoro-6-(3-hydroxyazetidin-1-yl)pyridin-3-yl]amino}pyrimidin-4-yl)-3,8-diazabicyclo[3.2.1]oct-8-yl]methanone;
[(1R,5S)-3-(2-{[5-chloro-6-(2-hydroxyethoxy)pyridin-3-yl]amino}pyrimidin-4-yl)-3,8-diazabicyclo[3.2.1]oct-8-yl][(1S)-2,2-difluorocyclopropyl]methanone;
{3-[(1R,5S)-3-{2-[(1-methyl-1H-pyrazol-4-yl)amino]pyrimidin-4-yl}-3,8-diazabicyclo[3.2.1]oct-8-yl]oxetan-3-yl}acetonitrile;
[(1R,5S)-3-(2-{[5-chloro-6-(2-hydroxyethyl)pyridin-3-yl]amino}pyrimidin-4-yl)-3,8-diazabicyclo[3.2.1]oct-8-yl][(1S)-2,2-difluorocyclopropyl]methanone;
2-[(1R,5S)-3-{2-[(1-methyl-1H-pyrazol-4-yl)amino]pyrimidin-4-yl}-3,8-diazabicyclo[3.2.1]oct-8-yl]pyridine-4-carbonitrile;
3-[(1R,5S)-3-{2-[(1-methyl-1H-pyrazol-4-yl)amino]pyrimidin-4-yl}-3,8-diazabicyclo[3.2.1]oct-8-yl]cyclobutanecarbonitrile;
2-[(1R,5S)-3-{2-[(1-methyl-1H-pyrazol-4-yl)amino]pyrimidin-4-yl}-3,8-diazabicyclo[3.2.1]oct-8-yl]-1,3-oxazole-5-carbonitrile;
(1R,5S)-N-(2-cyanoethyl)-3-{2-[(1-methyl-1H-pyrazol-4-yl)amino]pyrimidin-4-yl}-3,8-diazabicyclo[3.2.1]octane-8-carboxamide;
N-(1-methyl-1H-pyrazol-4-yl)-4-[(1R,5S)-8-(1,2-oxazol-4-ylmethyl)-3,8-diazabicyclo[3.2.1]oct-3-yl]pyrimidin-2-amine;
4-({4-[(1R,5S)-8-{[(1S)-2,2-difluorocyclopropyl]carbonyl}-3,8-diazabicyclo[3.2.1]oct-3-yl]pyrimidin-2-yl}amino)-6-(hydroxymethyl)-N-methylpyridine-2-carboxamide;
(1-fluorocyclopropyl)[(1R,5S)-3-{2-[(1-methyl-1H-pyrazol-4-yl)amino]pyrimidin-4-yl}-3,8-diazabicyclo[3.2.1]oct-8-yl]methanone;
N-(1-methyl-1H-pyrazol-4-yl)-4-[(1R,5S)-8-(1,3-thiazol-2-ylmethyl)-3,8-diazabicyclo[3.2.1]oct-3-yl]pyrimidin-2-amine;
cyclopropyl{(1R,5S)-3-[2-(1,2-thiazol-4-ylamino)pyrimidin-4-yl]-3,8-diazabicyclo[3.2.1]oct-8-yl}methanone;
[(1S)-2,2-difluorocyclopropyl]{(1R,5S)-3-[2-({5-fluoro-6-[(3R)-3-hydroxypyrrolidin-1-yl]pyridin-3-yl}amino)pyrimidin-4-yl]-3,8-diazabicyclo[3.2.1]oct-8-yl}methanone;
5-({4-[(1R,5S)-8-{[(1S)-2,2-difluorocyclopropyl]methyl}-3,8-diazabicyclo[3.2.1]oct-3-yl]pyrimidin-2-yl}amino)-N,3-dimethylpyridine-2-carboxamide;
4-[(1R,5S)-8-{[(1R)-2,2-difluorocyclopropyl]methyl}-3,8-diazabicyclo[3.2.1]oct-3-yl]-N-(1-methyl-1H-pyrazol-4-yl)pyrimidin-2-amine;
6-({4-[(1R,5S)-8-(cyclopropylcarbonyl)-3,8-diazabicyclo[3.2.1]oct-3-yl]-5-fluoropyrimidin-2-yl}amino)imidazo[1,2-a]pyridine-2-carboxamide;
5-({4-[(1R,5S)-8-(cyclopropylcarbonyl)-3,8-diazabicyclo[3.2.1]oct-3-yl]-5-fluoropyrimidin-2-yl}amino)pyridine-2-sulfonamide;
5-({4-[(1R,5S)-8-(trans-3-cyanocyclobutyl)-3,8-diazabicyclo[3.2.1]oct-3-yl]pyrimidin-2-yl}amino)-N,3-dimethylpyridine-2-carboxamide;
1,2-oxazol-5-yl{(1R,5S)-3-[2-(1H-pyrazol-4-ylamino)pyrimidin-4-yl]-3,8-diazabicyclo[3.2.1]oct-8-yl}methanone;
N-(1-methyl-1H-pyrazol-4-yl)-4-[(1R,5S)-8-(methylsulfonyl)-3,8-diazabicyclo[3.2.1]oct-3-yl]pyrimidin-2-amine;
(1S,2S)-2-{[(1R,5S)-3-{2-[(1-methyl-1H-pyrazol-4-yl)amino]pyrimidin-4-yl}-3,8-diazabicyclo[3.2.1]oct-8-yl]methyl}cyclopropanecarbonitrile;
3-({4-[(1R,5S)-8-(cyclopropylcarbonyl)-3,8-diazabicyclo[3.2.1]oct-3-yl]-5-fluoropyrimidin-2-yl}amino)-N-propyl-1H-pyrazole-5-carboxamide;
(1S,2S)-2-{[(1R,5S)-3-{2-[(1-methyl-1H-pyrazol-4-yl)amino]pyrimidin-4-yl}-3,8-diazabicyclo[3.2.1]oct-8-yl]methyl}cyclopropanecarbonitrile;
cyclopropyl{(1R,5S)-3-[5-fluoro-2-(pyridazin-4-ylamino)pyrimidin-4-yl]-3,8-diazabicyclo[3.2.1]oct-8-yl}methanone;
4-({4-[6-(2,2-difluoropropanoyl)-3,6-diazabicyclo[3.1.1]hept-3-yl]-5-fluoropyrimidin-2-yl}amino)-N-ethyl-2-methylbenzamide;
(1S,2S)-2-cyano-N-[(1S,5R,6R)-3-(2-{[6-(2-hydroxyethoxy)pyridin-3-yl]amino}-5-methylpyrimidin-4-yl)-6-methyl-3-azabicyclo[3.1.0]hex-1-yl]cyclopropanecarboxamide;
N-[(1S,5R)-3-(5-chloro-2-{[1-(2-hydroxyethyl)-1H-pyrazol-4-yl]amino}pyrimidin-4-yl)-3-azabicyclo[3.1.0]hex-1-yl]cyclopropanecarboxamide;
(1S)-2,2-difluoro-N-[(1S,5R,6R)-3-(5-fluoro-2-{[1-(oxetan-3-yl)-1H-pyrazol-4-yl]amino}pyrimidin-4-yl)-6-methyl-3-azabicyclo[3.1.0]hex-1-yl]cyclopropanecarboxamide;
(1S)-2,2-difluoro-N-[(1S,5S)-3-{5-fluoro-2-[(1-methyl-1H-pyrazol-4-yl)amino]pyrimidin-4-yl}-5-(hydroxymethyl)-3-azabicyclo[3.1.0]hex-1-yl]cyclopropanecarboxamide;
N-{(1S,5R,6R)-3-[5-fluoro-2-({6-[(2S)-1-hydroxypropan-2-yl]pyridin-3-yl}amino)pyrimidin-4-yl]-6-methyl-3-azabicyclo[3.1.0]hex-1-yl}cyclopropanecarboxamide;
5-[(4-{(1S,5R,6R)-1-[(cyclopropylcarbonyl)amino]-6-methyl-3-azabicyclo[3.1,0]hex-3-yl}-5-fluoropyrimidin-2-yl)amino]-N,3-dimethylpyridine-2-carboxamide;
N-{(1S,5R,6R)-3-[2-({5-chloro-6-[(1R)-1-hydroxyethyl]pyridin-3-yl}amino)-5-fluoropyrimidin-4-yl]-6-methyl-3-azabicyclo[3.1.0]hex-1-yl}cyclopropanecarboxamide;
(1R)-2,2-difluoro-N-[(1R,5S,6S)-3-{5-fluoro-2-[(1-methyl-1H-pyrazol-4-yl)amino]pyrimidin-4-yl}-6-methyl-3-azabicyclo[3.1.0]hex-1-yl]cyclopropanecarboxamide;
5-[(4-{(1R,5S,6S)-1-[(cyclopropylcarbonyl)amino]-6-methyl-3-azabicyclo[3.1.0]hex-3-yl}-5-fluoropyrimidin-2-yl)amino]-N,3-dimethylpyridine-2-carboxamide;
N-[(1R,5S)-3-(5-chloro-2-{[1-(2-hydroxyethyl)-1H-pyrazol-4-yl]amino}pyrimidin-4-yl)-3-azabicyclo[3.1.0]hex-1-yl]cyclopropanecarboxamide;
N-{(1S,5R,6R)-3-[5-fluoro-2-({6-[(2R)-1-hydroxypropan-2-yl]pyridin-3-yl}amino)pyrimidin-4-yl]-6-methyl-3-azabicyclo[3.1.0]hex-1-yl}cyclopropanecarboxamide;
(1S)-2,2-difluoro-N-[(1R,5S,6S)-3-{5-fluoro-2-[(1-methyl-1H-pyrazol-4-yl)amino]pyrimidin-4-yl}-6-methyl-3-azabicyclo[3.1.0]hex-1-yl]cyclopropanecarboxamide; and
((S)-2,2-difluorocyclopropyl)((1R,5S)-3-(2-((1-methyl-1H-pyrazol-4-yl)amino)pyrimidin-4-yl)-3,8-diazabicyclo[3.2.1]octan-8-yl)methanone; or, a pharmaceutically acceptable salt thereof.

In another embodiment, the present invention provides a method of treating a patient infected with SARS-CoV-2 comprising administering to the patient in need of such treatment a therapeutically effective amount of ((S)-2,2-difluorocyclopropyl)((1R,5S)-3-(2-((1-methyl-1H-pyrazol-4-yl)amino)pyrimidin-4-yl)-3,8-diazabicyclo[3.2.1]octan-8-yl)methanone p-toluenesulfonic acid salt.

In another embodiment, the present invention provides a method of treating a patient infected with SARS-CoV-2 comprising administering to the patient in need of such treatment a therapeutically effective amount of [(1R)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone or a pharmaceutically acceptable salt thereof.

In another embodiment, the present invention provides a method of treating a patient infected with SARS-CoV-2 comprising administering to the patient in need of such treatment a therapeutically effective amount of a compound selected from the group consisting of:
4-cyano-N-{cis-3-[methyl(7H-pyrrolo[2,3-d]pyrimidin-4-yl)amino]cyclobutyl}pyridine-2-sulfonamide;
2,2,2-trifluoro-N-{cis-3-[methyl(7H-pyrrolo[2,3-d]pyrimidin-4-yl)amino]cyclobutyl}-ethanesulfonamide;
2-methyl-N-{cis-3-[methyl(7H-pyrrolo[2,3-d]pyrimidin-4-yl)amino]cyclobutyl}-propane-1-sulfonamide;
N-{cis-3-[methyl(7H-pyrrolo[2,3-d]pyrimidin-4-yl)amino]cyclobutyl}propane-1-sulfonamide;
1-cyclopropyl-N-{cis-3-[methyl(7H-pyrrolo[2,3-d]pyrimidin-4-yl)amino]cyclobutyl}-methanesulfonamide;
N-{cis-3-[(butylsulfonyl)methyl]cyclobutyl}-N-methyl-7H-pyrrolo[2,3-d]pyrimidin-4-amine;
1-cyclopropyl-N-{cis-3-[methyl(7H-pyrrolo[2,3-d]pyrimidin-4-yl)amino]cyclobutyl}-azetidine-3-sulfonamide;
3-cyano-N-{cis-3-[methyl(7H-pyrrolo[2,3-d]pyrimidin-4-yl)amino]cyclobutyl}-azetidine-1-sulfonamide;
(1R5S)-N-{cis-3-[methyl(7H-pyrrolo[2,3-d]pyrimidin-4-yl)amino]cyclobutyl}-6-oxa-3-azabicyclo[3.1.1]heptane-3-sulfonamide;
(3R)-3-cyanoN-{cis-3-[methyl(7H-pyrrolo[2,3-d]pyrimidin-4-yl)amino]cyclobutyl}-pyrrolidine-1-sulfonamide;
(3S)-3-cyanoN-{cis-3-[methyl(7H-pyrrolo[2,3-d]pyrimidin-4-yl)amino]cyclobutyl}-pyrrolidine-1-sulfonamide;
N-cis-3-[methyl(7H-pyrrolo[2,3-d]pyrimidin-4-yl)amino]cyclobutyl}-1-(oxetan-3-yl)methane-sulfonamide;
1-(3,3-difluorocyclobutyl)-N-{cis-3-[methyl(7H-pyrrolo[2,3-d]pyrimidin-4-yl)amino]cyclobutyl}methane-sulfonamide;
trans-3-(cyanomethyl)-N-{cis-3-[methyl(7H-pyrrolo[2,3-d]pyrimidin-4-yl)amino]-cyclobutyl}cyclo-butanesulfonamide;
cis-3-(cyanomethyl)-N-{cis-3-[methyl(7H-pyrrolo[2,3-d]pyrimidin-4-yl)amino]-cyclobutyl}cyclobutane-sulfonamide;
N-[cis-3-({[(3,3-difluorocyclobutyl)methyl]sulfonyl}methyl)cyclobutyl]-N-methyl-7H-pyrrolo[2,3-d]pyrimidin-4-amine;
(1S,5S)-1-cyano-N-{cis-3-[methyl(7H-pyrrolo[2,3-d]pyrimidin-4-yl)amino]cyclobutyl}-3-azabicyclo[3.1.0]hexane-3-sulfonamide;
(1R,5R)-1-cyano-N-{cis-3-[methyl(7H-pyrrolo[2,3-d]pyrimidin-4-yl)amino]cyclobutyl}-3-azabicyclo[3.1.0]hexane-3-sulfonamide;
(3R)-1-[({cis-3-[methyl(7H-pyrrolo[2,3-d]pyrimidin-4-yl)amino]cyclobutyl}methyl)sulfonyl]pyrrolidine-3-carbonitrile;
1-[({cis-3-[methyl(7H-pyrrolo[2,3-d]pyrimidin-4-yl)amino]cyclobutyl)methyl)sulfonyl]-4-(trifluoromethyl)piperidin-4-ol;
N-(cis-3-{[(4,4-difluoropiperidin-1-yl)sulfonyl]methyl}cyclobutyl)-N-methyl-7H-pyrrolo[2,3-d]pyrimidin-4-amine;
(3S)-1-[({cis-3-[methyl(7H-pyrrolo[2,3-d]pyrimidin-4-yl)amino]cyclobutyl}meth-yl)sulfonyl]pyrrolidine-3-carbonitrile;
N-(cis-3-{[(3-chloro-4-fluorophenyl)sulfonyl]methyl}cyclobutyl)-N-methyl-7H-pyrrolo[2,3-d]pyrimidin-4-amine;
N-(cis-3-{[(2-cyclopropylethyl)sulfonyl]methyl}cyclobutyl)-N-methyl-7H-pyrrolo[2,3-d]pyrimidin-4-amine;
N-methyl-N-[cis-3-({[1-(propan-2-yl)pyrrolidin-3-yl]sulfonyl}methyl)cyclobutyl]-7H-pyrrolo[2,3-d]pyrimidin-4-amine;
3,3-difluoro-N-{cis-3-[methyl(7H-pyrrolo[2,3-d]pyrimidin-4-yl)amino]cyclobutyl}cyclobutane-sulfonamide;
1-[3-(cyanomethyl)oxetan-3-yl]-N-{cis-3-[methyl(7H-pyrrolo[2,3-d]pyrimidin-4-yl)amino]cyclobutyl}-methanesulfonamide;
cis-3-(cyanomethyl)-3-methyl-N-{cis-3-[methyl(7H-pyrrolo[2,3-d]pyrimidin-4-yl)amino]cyclobutyl}-cyclobutanesulfonamide;
trans-3-(cyanomethyl)-3-methyl-N-{cis-3-[methyl(7H-pyrrolo[2,3-d]pyrimidin-4-yl)amino]cyclobutyl}cyclobutanesulfonamide;
N-(2-cyanoethyl)-N-methyl-N′-{cis-3-[methyl(7H-pyrrolo[2,3-d]pyrimidin-4-yl)amino]cyclobutyl}sulfuric diamide;
N-{(1S,3R)-3-[methyl(7H-pyrrolo[2,3-d]pyrimidin-4-yl)amino]cyclopentyl}propane-1-sulfonamide;
3-(2-hydroxypropan-2-yl)-N-{cis-3-[methyl(7H-pyrrolo[2,3-d]pyrimidin-4-yl)amino]cyclobutyl}benzene-sulfonamide;
N-(cyclopropylmethyl)-N′-{cis-3-[methyl(7H-pyrrolo[2,3-d]pyrimidin-4-yl)amino]cyclobutyl}sulfuric diamide;
N-{cis-3-[methyl(7H-pyrrolo[2,3-d]pyrimidin-4-yl)amino]cyclobutyl}-4-(1H-pyrazol-3-yl)piperidine-1-sulfonamide;
2-methyl-N-{cis-3-[methyl(7H-pyrrolo[2,3-d]pyrimidin-4-yl)amino]cyclobutyl}-2,6-dihydropyrrolo[3,4-c]pyrazole-5(4H)-sulfonamide;
N-cyclopropyl-1-{trans-3-[methyl(7H-pyrrolo[2,3-d]pyrimidin-4-yl)amino]cyclobutyl}methane-sulfonamide;
2-[({cis-3-[methyl(7H-pyrrolo[2,3-d]pyrimidin-4-yl)amino]cyclobutyl}methyl)sulfonyl]pyridine-4-carbonitrile;
(1S,3S)-3-[({cis-3-[methyl(7H-pyrrolo[2,3-d]pyrimidin-4-yl)amino]cyclobutyl}methyl)-sulfonyl]cyclopentanecarbonitrile;
(1R,3R)-3-[({cis-3-[methyl(7H-pyrrolo[2,3-d]pyrimidin-4-yl)amino]cyclobutyl}methyl)sulfonyl] cyclopentanecarbonitrile;
1-cyclopropyl-N-{trans-3-[methyl(7H-pyrrolo[2,3-d]pyrimidin-4-yl)amino]cyclobutyl}methane sulfonamide;
3-cyano-N-{trans-3-[methyl(7H-pyrrolo[2,3-d]pyrimidin-4-yl)amino]cyclobutyl}pyrrolidine-1-sulfonamide;
N-methyl-N-{trans-3-[(propylsulfonyl)methyl]cyclobutyl}-7H-pyrrolo[2,3-d]pyrimidin-4-amine; and,
2-methyl-N-{cis-3-[methyl(7H-pyrrolo[2,3-d]pyrimidin-4-yl)amino]cyclobutyl}-1,3-thiazole-5-sulfonamide; or, a pharmaceutically acceptable salt thereof.

In another embodiment, the present invention provides a method of treating a patient infected with SARS-CoV-2 comprising administering orally to the patient in need of such treatment a therapeutically effective amount of N-{cis-3-[methyl(7H-pyrrolo[2,3-d]pyrimidin-4-yl)amino]cyclobutyl}-propane-1-sulfonamide, or a pharmaceutically acceptable salt thereof.

In another embodiment, the present invention provides a method of treating a patient infected with SARS-CoV-2 comprising administering orally to the patient in need of such treatment a therapeutically effective amount of a compound selected from the group consisting of:
(1r,3r)-3-(4-(6-(3-amino-1-methyl-1H-pyrazol-4-yl)pyrazolo[1,5-a]pyrazin-4-yl)-1H-pyrazol-1-yl)-3-(cyanomethyl)cyclobutane-1-carbonitrile;
2,2′-(3-(4-(6-(1-methyl-1H-pyrazol-4-yl)pyrazolo[1,5-a]pyrazin-4-yl)-1H-pyrazol-1-yl)azetidine-1,3-diyl)diacetonitrile;
2-((1s,3r)-1-(4-(6-(5-(hydroxymethyl)-1H-pyrazol-3-yl)pyrazolo[1,5-a]pyrazin-4-yl)-1H-pyrazol-1-yl)-3-methoxycyclobutyl)acetonitrile;
5-(4-(1-((1s,3r)-1-(cyanomethyl)-3-methoxycyclobutyl)-1H-pyrazol-4-yl)pyrazolo[1,5-a]pyrazin-6-yl)-1H-pyrazole-3-carboxamide;
(1s,3s)-3-(cyanomethyl)-3-(4-(6-(5-(hydroxymethyl)isoxazol-3-yl)pyrazolo[1,5-a]pyrazin-4-yl)-1H-pyrazol-1-yl)cyclobutane-1-carbonitrile;
(1r,3r)-3-(cyanomethyl)-3-(4-(6-(1-methyl-1H-pyrazol-4-yl)pyrazolo[1,5-a]pyrazin-4-yl)-1H-pyrazol-1-yl)cyclobutane-1-carbonitrile;
(1s,3s)-3-(cyanomethyl)-3-(4-(6-(1-methyl-1H-pyrazol-4-yl)pyrazolo[1,5-a]pyrazin-4-yl)-1H-pyrazol-1-yl)cyclobutane-1-carbonitrile;
(1r,3r)-3-(cyanomethyl)-3-(4-(3-methyl-6-(1-methyl-1H-pyrazol-4-yl)pyrazolo[1,5-a]pyrazin-4-yl)-1H-pyrazol-1-yl)cyclobutane-1-carbonitrile;
2-((1r,3s)-1-(4-(6-(3-amino-1H-pyrazol-5-yl)pyrazolo[1,5-a]pyrazin-4-yl)-1H-pyrazol-1-yl)-3-methoxycyclobutyl)acetonitrile;
2-(1-ethyl-3-(4-(6-(5-(hydroxymethyl)-1H-pyrazol-3-yl)pyrazolo[1,5-a]pyrazin-4-yl)-1H-pyrazol-1-yl)azetidin-3-yl)acetonitrile;
(1r,3r)-3-(cyanomethyl)-3-(4-(6-(1-methyl-3-oxo-2,3-dihydro-1H-pyrazol-4-yl)pyrazolo[1,5-a]pyrazin-4-yl)-1H-pyrazol-1-yl)cyclobutane-1-carbonitrile (trans isomer); and
(1r,3r)-3-(cyanomethyl)-3-(4-(6-(1-(hydroxymethyl)-1H-pyrazol-4-yl)pyrazolo[1 ,5-a]pyrazin-4-yl)-1H-pyrazol-1-yl)cyclobutane-1-carbonitrile; or a pharmaceutically acceptable salt thereof.

In another embodiment, the present invention provides a method of treating a patient infected with SARS-CoV-2 comprising administering to the patient in need of such treatment a therapeutically effective amount of (1r,3r)-3-(cyanomethyl)-3-(4-(6-(1-methyl-1H-pyrazol-4-yl)pyrazolo[1,5-a]pyrazin-4-yl)-1H-pyrazol-1-yl)cyclobutane-1-carbonitrile or a pharmaceutically acceptable salt thereof.

In another embodiment, the present invention provides a method of treating a patient infected with SARS-CoV-2 comprising administering to the patient in need of such treatment a therapeutically effective amount of (1s,3s)-3-(cyanomethyl)-3-(4-(6-(1-methyl-1H-pyrazol-4-yl)pyrazolo[1,5-a]pyrazin-4-yl)-1H-pyrazol-1-yl)cyclobutane-1-carbonitrile or a pharmaceutically acceptable salt thereof.

In another embodiment, the present invention provides a method of treating a patient infected with SARS-CoV-2 comprising administering to the patient in need of such treatment a therapeutically effective amount of a compound selected from the group consisting of ruxolitinib, baricitinib, oclacitinib, fedratinib, upadacitinib and peficitinib.

In another embodiment, the present invention provides a method of treating a patient infected with SARS-CoV-2 comprising administering to the patient in need of such treatment a therapeutically effective amount of a compound selected from the group consisting of filgotinib, decernotinib, cerdulatinib, gandotinib, lestaurtinib, momelotinib, and pacritinib.

In another embodiment, the present invention provides a method of treating a patient infected with SARS-CoV-2 comprising administering to the patient in need of such treatment a therapeutically effective amount of a compound selected from the group consisting of adalimumab, infliximab, certolizumab, golimumab, and vedolizumab.

In another embodiment, the present invention provides a method of treating a patient infected with SARS-CoV-2 comprising administering to the patient in need of such treatment a therapeutically effective amount of etanercept.

In another embodiment, the present invention provides a method of treating a patient infected with SARS-CoV-2 comprising administering to the patient in need of such treatment a therapeutically effective amount of a compound selected from the group consisting of abrilada, hadlima, hyrimoz, cyltezo, and Amjevita.

In another embodiment, the present invention provides a method of treating a patient infected with SARS-CoV-2 comprising administering to the patient in need of such treatment a therapeutically effective amount of a compound selected from the group consisting of cucurbitacin, CHZ868, GLPG-0634, INCB-47986, INCB-039110, XL-019, ABT-494, R-348, GSK-2586184, AC-410, BMS-911543, and JTE-052.

In another embodiment, the present invention provides a method of treating a patient infected with SARS-CoV-2 comprising administering to the patient in need of such treatment a therapeutically effective amount of a compound selected from the group consisting of: 4-(1-(1-ethoxyethyl)-1H-pyrazol-4-yl)-6-(1-methyl-1Hpyrazol-4-yl)pyrazolo[1,5-a]pyrazine; 4-(1-cycloheptyl-1H-pyrazol-4-yl)-6-(1-methyl-1H-pyrazol-4-yl)pyrazolo[1,5-a]pyrazine; 4-(2-(4-(4-(1-(pentan-3-yl)-1H-pyrazol-4-yl)pyrazolo[1,5-a]pyrazin-6-yl)-1H-pyrazol-1-yl)ethyl)morpholine; 4-(1-(pentan-3-yl)-1H-pyrazol-4-yl)-6-(1-((tetrahydro-2H-pyran-4-yl)methyl)-1H-pyrazol-4-yl)pyrazolo[1,5-a]pyrazine; N,N-dimethyl-2-(4-(4-(1-(pentan-3-yl)-1H-pyrazol-4-yl)pyrazolo[1,5-a]pyrazin-6-yl)-1H-pyrazol-1-yl)acetamide; 1-morpholino-2-(4-(4-(1-(pentan-3-yl)-1H-pyrazol-4-yl)pyrazolo[1,5-a]pyrazin-6-yl)-1H-pyrazol-1-yl)ethanone; 6-(1-(3-(methylsulfonyl)propyl)-1 H-pyrazol-4-yl)-4-(1-(pentan-3-yl)-1H-pyrazol-4-yl)pyrazolo[1,5-a]pyrazine; 5-((4-(4-(1-(pentan-3-yl)-1H-pyrazol-4-yl)pyrazolo[1,5-a]pyrazin-6-yl)-1H-pyrazol-1-yl)methyl)oxazolidin-2-one; N-methyl-N-(2-(4-(4-(1-(pentan-3-yl)-1H-pyrazol-4-yl)pyrazolo[1,5-a]pyrazin-6-yl)-1H-pyrazol-1-yl)ethyl)methanesulfonamide; N,N-dimethyl-2-(4-(4-(1-(pentan-3-yl)-1H-pyrazol-4-yl)pyrazolo[1,5-a]pyrazin-6-yl)-1H-pyrazol-1-yl)ethanamine; 4-((4-(4-(1-(pentan-3-yl)-1H-pyrazol-4-yl)pyrazolo[1,5-a]pyrazin-6-yl)-1H-pyrazol-1-yl)methyl)tetrahydro-2H-thiopyran 1,1-dioxide; N,N-dimethyl-3-(4-(4-(1-(pentan-3-yl)-1H-pyrazol-4-yl)pyrazolo[1,5-a]pyrazin-6-yl)-1H-pyrazol-1-yl)propan-1-amine; 3-(4-(4-(1-(pentan-3-yl)-1H-pyrazol-4-yl)pyrazolo[1,5-a]pyrazin-6-yl)-1H-pyrazol-1-yl)thietane 1,1-dioxide; (R)-2-methyl-3-(4-(4-(1-(pentan-3-yl)-1H-pyrazol-4-yl)pyrazolo[1 ,5-a]pyrazin-6-yl)-1H-pyrazol-1-yl)propan-1-ol; (S)-2-methyl-3-(4-(4-(1-(pentan-3-yl)-1H-pyrazol-4-yl)pyrazolo[1,5-a]pyrazin-6-yl)-1H-pyrazol-1-yl)propan-1-ol; (3-((4-(4-(1-(pentan-3-yl)-1H-pyrazol-4-yl)pyrazolo[1,5-a]pyrazin-6-yl)-1-H-pyrazol-1-yl)methyl)oxetan-3-yl)methanol; (S)-5-((4-(4-(1-(pentan-3-yl)-1H-pyrazol-4-yl)pyrazolo[1,5-a]pyrazin-6-yl)-1H-pyrazol-1-yl)methyl)pyrrolidin-2-one; (R)-5-((4-(4-(1-(pentan-3-yl)-1H-pyrazol-4-yl)pyrazolo[1,5-a]pyrazin-6-yl)-1H-pyrazol-1-yl)methyl)pyrrolidin-2-one; 3-(4-(4-(1-(pentan-3-yl)-1H-pyrazol-4-yl)pyrazolo[1,5-a]pyrazin-6-yl)-1 H-pyrazol-1-yl)propan-1-ol; 2-(4-(4-(1-(pentan-3-yl)-1H-pyrazol-4-yl)pyrazolo[1,5-a]pyrazin-6-yl)-1H-pyrazol-1-yl)ethanol; (R)-4-(4-(4-(1-(sec-butyl)-1H-pyrazol-4-yl)pyrazolo[1,5-a]pyrazin-6-yl)-1-H-pyrazol-1-yl)tetrahydro-2H-thiopyran 1,1-dioxide; 6-(1-(2-(methylsulfonyl)ethyl)-1H-pyrazol-4-yl)-4-(1-(pentan-3-yl)-1H-pyrazol-4-yl)pyrazolo[1,5-a]pyrazine; N,N-dimethyl-2-(4-(4-(1-(pentan-3-yl)-1H-pyrazol-4-yl)pyrazolo[1,5-a]pyrazin-6-yl)-1 H-pyrazol-1-yl)ethanesulfonamide; 2-(4-(4-(1-(pentan-3-yl)-1H-pyrazol-4-yl)pyrazolo[1,5-a]pyrazin-6-yl)-1H-pyrazol-1-yl)ethanesulfonamide; 4-(1-(pentan-3-yl)-1H-pyrazol-4-yl)-6-(1-(piperidin-4-yl)-1H-pyrazol-4-yl)pyrazolo[1,5-a]pyrazine; 4-(1-isopropyl-1H-pyrazol-4-yl)-6-(1-(piperidin-4-yl)-1H-pyrazol-4-yl)pyrazlo[1,5-a]pyrazine; (R)-4-(1-(pentan-3-yl)-1H-pyrazol-4-yl)-6-(1-(pyrrolidin-2-ylmethyl)-1H-pyrazol-4-yl)pyrazolo[1,5-a]pyrazine; (S)-4-(1-(pentan-3-yl)-1H-pyrazol-4-yl)-6-(1-(pyrrolidin-2-ylmethyl)-1H-pyrazol-4-yl)pyrazolo[1,5-a]pyrazine; (R)-4-(1-(pentan-3-yl)-1H-pyrazol-4-yl)-6-(1-(piperidin-3-yl)-1H-pyrazol-4-yl)pyrazolo[1,5-a]pyrazine; (S)-3-((4-(4-(1-(pentan-3-yl)-1H-pyrazol-4-yl)pyrazolo[1,5-a]pyrazin-6-yl)-1H-pyrazol-1-yl)methyl)morpholine; 1-(4-(4-(4-(1-(pentan-3-yl)-1H-pyrazol-4-yl)pyrazolo[1,5-a]pyrazin-6-yl)-1H-pyrazol-1-yl)piperidin-1-yl)ethanone; 6-(1-(1-(methylsulfonyl)piperidin-4-yl)-1H-pyrazol-4-yl)-4-(1-(pentan-3-y-l)-1H-pyrazol-4-yl)pyrazolo[1,5-a]pyrazine; 2-methoxy-1-(4-(4-(4-(1-(pentan-3-yl)-1H-pyrazol-4-yl)pyrazolo[1,5-a]pyrazin-6-yl)-1H-pyrazol-1-yl)piperidin-1-yl)ethanone; N-methyl-4-(4-(4-(1-(pentan-3-yl)-1H-pyrazol-4-yl)pyrazolo[1,5-a]pyrazin-6-yl)-1H-pyrazol-1-yl)piperidine-1-carboxamide; N,N-dimethyl-4-(4-(4-(1-(pentan-3-yl)-1H-pyrazol-4-yl)pyrazolo[1,5-a]pyrazin-6-yl)-1H-pyrazol-1-yl)piperidine-1-carboxamide; 2-amino-1-(4-(4-(4-(1-(pentan-3-yl)-1H-pyrazol-4-yl)pyrazolo[1,5-a]pyrazine-6-yl)-1H-pyrazol-1-yl)piperidin-1-yl)ethanone; 6-(1-(1-(methylsulfonyl)azetidin-3-yl)-1H-pyrazol-4-yl)-4-(1-(pentan-3-yl)-1H-pyrazol-4-yl)pyrazolo[1,5-a]pyrazine; 1-(3-(4-(4-(1-(pentan-3-yl)-1H-pyrazol-4-yl)pyrazolo[1 ,5-a]pyrazin-6-yl)-1H-pyrazol-1-yl)azetidin-1-yl)ethanone; N-methyl-3-(4-(4-(1-(pentan-3-yl)-1H-pyrazol-4-yl)pyrazolo[1,5-a]pyrazin-6-yl)-1H-pyrazol-1-yl)azetidine-1-carboxamide; N,N-dimethyl-3-(4-(4-(1-(pentan-3-yl)-1H-pyrazol-4-yl)pyrazolo[1,5-a]pyrazin-6-yl)-1H-pyrazol-1-yl)azetidine-1-carboxamide; Bis-N,N-dimethyl-P-(3-(4-(4-(1-(pentan-3-yl)-1H-pyrazol-4-yl)pyrazolo[1,5-a]pyrazin-6-yl)-1H-pyrazol-1-yl)azetidin-1-yl)phosphonic amide; 2-methyl-1-(3-(4-(4-(1-(pentan-3-yl)-1H-pyrazol-4-yl)pyrazolo[1,5-a]pyrazin-6-yl)-1H-pyrazol-1-yl)azetidin-1-yl)propan-1-one; cyclopropyl(3-(4-(4-(1-(pentan-3-yl)-1H-pyrazol-4-yl)pyrazolo[1,5-a]pyrazin-6-yl)-1H-pyrazol-1-yl)azetidin-1-yl)methanone; 2-(4-(6-(1-methyl-1H-pyrazol-4-yl)pyrazolo[1,5-a]pyrazine-4-yl)-1H-pyrazol-1-yl)butanoic acid; 2-(4-(6-(1-methyl-1H-pyrazol-4-yl)pyrazolo[1 ,5-a]pyrazin-4-yl)-1H-pyrazol-1-yl)butan-1-ol; 2-(4-(6-(1-methyl-1H-pyrazol-4-yl)pyrazolo[1,5-a]pyrazin-4-yl)-1H-pyrazol-1-yl)propan-1-ol; 3-(4-(6-(1-methyl-1 H-pyrazol-4-yl)pyrazolo[1,5-a]pyrazin-4-yl)-1H-pyrazol-1-yl)pentan-1-ol; 4-(1-(3-ethyl-1-((trifluoromethyl)sulfonyl)azetidin-3-yl)-1H-pyrazol-4-yl)-6-(1-methyl-1H-pyrazol-4-yl)pyrazolo[1,5-a]pyrazine; 6-(1-methyl-1H-pyrazol-4-yl)-4-(1-(1-methylcyclopentyl)-1H-pyrazol-4-yl)p-yrazolo[1,5-a]pyrazine; 6-(1-methyl-1H-pyrazol-4-yl)-4-(1-(3-methylpentan-3-yl)-1H-pyrazol-4-yl)pyrazolo[1,5-a]pyrazine; (2-(4-(6-(1-methyl-1H-pyrazol-4-yl)pyrazolo[1,5-a]pyrazin-4-yl)-1H-pyrazol-1-yl)cyclopentyl)methanol; 6-(1-methyl-1H-pyrazol-4-yl)-4-(1-(2-methylcycloheptyl)-1H-pyrazol-4-yl)pyrazolo[1,5-a]pyrazine; 2-(4-(6-(1-methyl-1H-pyrazol-4-yl)pyrazolo[1,5-a]pyrazin-4-yl)-1H-pyrazol-1-yl)cyclopentanol; (R)-3-(4-(4-(1-(pentan-3-yl)-1H-pyrazol-4-yl)pyrazolo[1,5-a]pyrazin-6-yl)-1H-pyrazol-1-yl)propane-1 ,2-diol; (S)-3-(4-(4-(1-(pentan-3-yl)-1H-pyrazol-4-yl)pyrazolo[1,5-a]pyrazin-6-yl)-1H-pyrazol-1-yl)propane-1 ,2-diol; (S)-3-methyl-1-(4-(4-(1-(pentan-3-yl)-1H-pyrazol-4-yl)pyrazolo[1,5-a]pyrazin-6-yl)-1H-pyrazol-1-yl)butane-2,3-diol; (R)-3-methyl-1-(4-(4-(1-(pentan-3-yl)-1H-pyrazol-4-yl)pyrazolo[1,5-a]pyrazin-6-yl)-1H-pyrazol-1-yl)butane-2,3-diol; 3-(4-(4-(1-(pentan-3-yl)-1H-pyrazol-4-yl)pyrazolo[1,5-a]pyrazin-6-yl)-1H-pyrazol-1-yl)propane-1,2-diol; (2R,3R)-1-(4-(4-(1-(pentan-3-yl)-1H-pyrazol-4-yl)pyrazolo[1,5-a]pyrazin-6-yl)-1H-pyrazol-1-yl)butane-2,3-diol; 2-methyl-3-(4-(4-(1-(pentan-3-yl)-1H-pyrazol-4-yl)pyrazolo[1,5-a]pyrazin-6-yl)-1H-pyrazol-1-yl)propane-1,2-diol; 2-((4-(4-(1-(pentan-3-yl)-1H-pyrazol-4-yl)pyrazolo[1,5-a]pyrazin-6-yl)-1H- -pyrazol-1-yl)methyl)propane-1,3-diol; (S)-4-(4-(4-(1-(pentan-3-yl)-1H-pyrazol-4-yl)pyrazolo[1,5-a]pyrazin-6-yl)-1H-pyrazol-1-yl)butane-1,2-diol; 4-(4-(4-(1-(pentan-3-yl)-1H-pyrazol-4-yl)pyrazolo[1,5-a]pyrazin-6-yl)-1H-pyrazol-1-yl)butane-1,3-diol; (R)-4-(4-(4-(1-(pentan-3-yl)-1H-pyrazol-4-yl)pyrazolo[1,5-a]pyrazin-6-yl)-1H-pyrazol-1-yl)butane-1,2-diol; (2S,3S)-1-(4-(4-(1-(pentan-3-yl)-1H-pyrazol-4-yl)pyrazolo[1,5-a]pyrazin-6-yl)-1H-pyrazol-1-yl)butane-2,3-diol; (2R)-3-(4-(4-(1-(1-phenylpropyl)-1H-pyrazol-4-yl)pyrazolo[1,5-a]pyrazin-6-yl)-1H-pyrazol-1-yl)propane-1,2-diol; (2R)-3-(4-(4-(1-(sec-butyl)-1H-pyrazol-4-yl)pyrazolo[1,5-a]pyrazin-6-yl)-1H-pyrazol-1-yl)propane-1,2-diol; (R)-3-(4-(4-(1-((S)-sec-butyl)-1H-pyrazol-4-yl)pyrazolo[1,5-a]pyrazin-6-yl)-1H-pyrazol-1-yl)propane-1,2-diol; (R)-3-(4-(4-(1-((R)-sec-butyl)-1H-pyrazol-4-yl)pyrazolo[1,5-a]pyrazin-6-yl)-1H-pyrazol-1-yl)propane-1,2-diol; (S)-3-(4-(4-(1-((S)-sec-butyl)-1H-pyrazol-4-yl)pyrazolo[1,5-a]pyrazin-6-yl)-1H-pyrazol-1-yl)propane-1,2-diol; (S)-3-(4-(4-(1-((R)-sec-butyl)-1H-pyrazol-4-yl)pyrazolo[1,5-a]pyrazin-6-yl)-1H-pyrazol-1-yl)propane-1 ,2-diol; (R)-3-(4-(4-(1-((S)-pentan-2-yl)-1H-pyrazol-4-yl)pyrazolo[1,5-a]pyrazin-6-yl)-1H-pyrazol-1-yl)propane-1,2-diol; (R)-3-(4-(4-(1-((R)-pentan-2-yl)-1H-pyrazol-4-yl)pyrazolo[1,5-a]pyrazin-6-yl)-1H-pyrazol-1-yl)propane-1,2-diol; (S)-3-(4-(4-(1-((R)-pentan-2-yl)-1H-pyrazol-4-yl)pyrazolo[1,5-a]pyrazin-6-yl)-1H-pyrazol-1-yl)propane-1,2-diol; (R)-3-(4-(4-(1-((S)-4-methylpentan-2-yl)-1H-pyrazol-4-yl)pyrazolo[1,5-a]pyrazin-6-yl)-1H-pyrazol-1-yl)propane-1,2-diol; (S)-3-(4-(4-(1-((S)-4-methylpentan-2-yl)-1H-pyrazol-4-yl)pyrazolo[1,5-a]pyrazin-6-yl)-1H-pyrazol-1-yl)propane-1,2-diol; (R)-3-(4-(4-(1-((R)-4-methylpentan-2-yl)-1H-pyrazol-4-yl)pyrazolo[1,5-a]pyrazin-6-yl)-1H-pyrazol-1-yl)propane-1,2-diol; (S)-3-(4-(4-(1-((R)-4-methylpentan-2-yl)-1H-pyrazol-4-yl)pyrazolo[1,5-a]pyrazin-6-yl)-1H-pyrazol-1-yl)propane-1,2-diol; (2R)-3-(4-(4-(1-(1-(3,3-difluorocyclobutyl)propyl)-1H-pyrazol-4-yl)pyrazolo[1,5-a]pyrazin-6-yl)-1H-pyrazol-1-yl)propane-1,2-diol; (R)-3-(4-(4-(1-((S)-3-methylbutan-2-yl)-1H-pyrazol-4-yl)pyrazolo[1,5-a]pyrazin-6-yl)-1H-pyrazol-1-yl)propane-1,2-diol; 2-(4-(4-(1-(pentan-3-yl)-1H-pyrazol-4-yl)pyrazolo[1 ,5-a]pyrazin-6-yl)-1H-pyrazol-1-yl)propane-1,3-diol; (S)-2-(4-(4-(1-(pentan-2-yl)-1H-pyrazol-4-yl)pyrazolo[1,5-a]pyrazin-6-yl)-1H-pyrazol-1-yl)propane-1,3-diol; (R)-2-(4-(4-(1-(pentan-2-yl)-1H-pyrazol-4-yl)pyrazolo[1,5-a]pyrazin-6-yl)-1H-pyrazol-1-yl)propane-1,3-diol; (S)-2-(4-(4-(1-(sec-butyl)-1H-pyrazol-4-yl)pyrazolo[1,5-a]pyrazin-6-yl)-1-H-pyrazol-1-yl)propane-1,3-diol; (R)-2-(4-(4-(1-(sec-butyl)-1H-pyrazol-4-yl)pyrazolo[1,5-a]pyrazin-6-yl)-1-H-pyrazol-1-yl)propane-1,3-diol; (S)-3-(4-(4-(1-((S)-pentan-2-yl)-1H-pyrazol-4-yl)pyrazolo[1,5-a]pyrazin-6-yl)-1H-pyrazol-1-yl)propane-1,2-diol; (S)-2-(4-(4-(1-(4-methylpentan-2-yl)-1H-pyrazol-4-yl)pyrazolo[1,5-a]pyrazin-6-yl)-1H-pyrazol-1-yl)propane-1,3-diol; (R)-2-(4-(4-(1-(4-methylpentan-2-yl)-1H-pyrazol-4-yl)pyrazolo[1,5-a]pyrazin-6-yl)-1H-pyrazol-1-yl)propane-1,3-diol; (2S,3S)-3-(4-(4-(1-(pentan-3-yl)-1H-pyrazol-4-yl)pyrazolo[1,5-a]pyrazin-6-yl)-1H-pyrazol-1-yl)butane-1,2-diol; (2S,3R)-3-(4-(4-(1-(pentan-3-yl)-1H-pyrazol-4-yl)pyrazolo[1,5-a]pyrazin-6-yl)-1H-pyrazol-1-yl)butane-1,2-diol; (2R,3S)-3-(4-(4-(1-(pentan-3-yl)-1H-pyrazol-4-yl)pyrazolo[1 ,5-a]pyrazin-6-yl)-1H-pyrazol-1-yl)butane-1 ,2-diol; (2R,3R)-3-(4-(4-(1-(pentan-3-yl)-1H-pyrazol-4-yl)pyrazolo[1 ,5-a]pyrazin-6-yl)-1H-pyrazol-1-yl)butane-1 ,2-diol; (R)-3-(4-(4-(1-((1R,2S)-2-methylcyclopentyl)-1 H-pyrazol-4-yl)pyrazolo[1 ,5-a]pyrazin-6-yl)-1H-pyrazol-1-yl)propane-1 ,2-diol; (R)-3-(4-(4-(1-((1S,2R)-2-methylcyclopentyl)-1H-pyrazol-4-yl)pyrazolo[1 ,5-a]pyrazin-6-yl)-1H-pyrazol-1-yl)propane-1 ,2-diol; (R)-3-(4-(4-(1-((S)-2,2-dimethylcyclopentyl)-1H-pyrazol-4-yl)pyrazolo[1 ,5-a]pyrazin-6-yl)-1H-pyrazol-1-yl)propane-1 ,2-diol; (R)-3-(4-(4-(1-((R)-2,2-dimethylcyclopentyl)-1H-pyrazol-4-yl)pyrazolo[1 ,5-a]pyrazin-6-yl)-1 H-pyrazol-1-yl)propane-1 ,2-diol; N-isopropyl-2-(4-(4-(1-(pentan-3-yl)-1H-pyrazol-4-yl)pyrazolo[1 ,5-a]pyrazin-6-yl)-1 H-pyrazol-1-yl)acetamide; 1-amino-3-(4-(4-(1-(pentan-3-yl)-1H-pyrazol-4-yl)pyrazolo[1 ,5-a]pyrazin-6-yl)-1H-pyrazol-1-yl)propan-2-ol; (R)-1-(dimethylamino)-3-(4-(4-(1-(pentan-3-yl)-1 H-pyrazol-4-yl)pyrazolo[1 ,5-a]pyrazin-6-yl)-1H-pyrazol-1-yl)propan-2-ol; (R)-1-(methylamino)-3-(4-(4-(1-(pentan-3-yl)-1H-pyrazol-4-yl)pyrazolo[1 ,5-a]pyrazin-6-yl)-1H-pyrazol-1-yl)propan-2-ol; (S)-1-(dimethylamino)-3-(4-(4-(1-(pentan-3-yl)-1H-pyrazol-4-yl)pyrazolo[1 ,5-a]pyrazin-6-yl)-1 H-pyrazol-1-yl)propan-2-ol; (S)-1-(methylamino)-3-(4-(4-(1-(pentan-3-yl)-1H-pyrazol-4-yl)pyrazolo[1 ,5-a]pyrazin-6-yl)-1H-pyrazol-1-yl)propan-2-ol; (R)-1-(3-methoxyazetidin-1-yl)-3-(4-(4-(1-(pentan-3-yl)-1H-pyrazol-4-yl)p- yrazolo[1 ,5-a]pyrazin-6-yl)-1H-pyrazol-1-yl)propan-2-ol; (S)-1-(3-methoxyazetidin-1-yl)-3-(4-(4-(1-(pentan-3-yl)-1H-pyrazol-4-yl)p- yrazolo[1 ,5-a]pyrazin-6-yl)-1H-pyrazol-1-yl)propan-2-ol; (R)-1-(4-(4-(1-(pentan-3-yl)-1H-pyrazol-4-yl)pyrazolo[1 ,5-a]pyrazin-6-yl)-1H-pyrazol-1-yl)-3-(pyrrolidin-1-yl)propan-2-ol; (S)-1-(4-(4-(1-(pentan-3-yl)-1H-pyrazol-4-yl)pyrazolo[1 ,5-a]pyrazin-6-yl)-1H-pyrazol-1-yl)-3-(pyrrolidin-1-yl)propan-2-ol; (R)-1-methoxy-3-(4-(4-(1-(pentan-3-yl)-1H-pyrazol-4-yl)pyrazolo[1 ,5-a]pyrazin-6-yl)-1H-pyrazol-1-yl)propan-2-ol; (S)-1-methoxy-3-(4-(4-(1-(pentan-3-yl)-1H-pyrazol-4-yl)pyrazolo[1 ,5-a]pyrazin-6-yl)-1H-pyrazol-1-yl)propan-2-ol; (R)-1-(4-(4-(1-(pentan-3-yl)-1H-pyrazol-4-yl)pyrazolo[1 ,5-a]pyrazin-6-yl)-1H-pyrazol-1-yl)propan-2-ol; (S)-1-(4-(4-(1-(pentan-3-yl)-1H-pyrazol-4-yl)pyrazolo[1 ,5-a]pyrazin-6-yl)-1H-pyrazol-1-yl)propan-2-ol; 4,4,4-trifluoro-1-(4-(4-(1-(pentan-3-yl)-1H-pyrazol-4-yl)pyrazolo[1,5-a]pyrazin-6-yl)-1H-pyrazol-1-yl)butan-2-ol; 3,3-dimethyl-1-(4-(4-(1-(pentan-3-yl)-1H-pyrazol-4-yl)pyrazolo[1 ,5-a]pyrazin-6-yl)-1H-pyrazol-1-yl)butan-2-ol; 3-methyl-1-(4-(4-(1-(pentan-3-yl)-1H-pyrazol-4-yl)pyrazolo[1 ,5-a]pyrazin-6-yl)-1H-pyrazol-1-yl)butan-2-ol; (S)-1-(4-(4-(1-(pentan-3-yl)-1H-pyrazol-4-yl)pyrazolo[1,5-a]pyrazin-6-yl)-1H-pyrazol-1 -yl)butan-2-ol; (R)-1-(4-(4-(1-(pentan-3-yl)-1H-pyrazol-4-yl)pyrazolo[1 ,5-a]pyrazin-6-yl)-1H-pyrazol-1-yl)butan-2-ol; 4-((4-(4-(1-(pentan-3-yl)-1H-pyrazol-4-yl)pyrazolo[1 ,5-a]pyrazin-6-yl)-1H- -pyrazol-1-yl)methyl)tetrahydro-2H-pyran-4-ol; 2-methyl-1-(4-(4-(1-(pentan-3-yl)-1H-pyrazol-4-yl)pyrazolo[1 ,5-a]pyrazin-6-yl)-1H-pyrazol-1-yl)propan-2-ol; trans-4-(4-(4-(1-(pentan-3-yl)-1H-pyrazol-4-yl)pyrazolo[1 ,5-a]pyrazin-6-yl)-1H-pyrazol-1-yl)cyclohexanol; cis-3-(4-(4-(1-(pentan-3-yl)-1H-pyrazol-4-yl)pyrazolo[1 ,5-a]pyrazin-6-yl)-1H-pyrazol-1 -yl)cyclobutanol; (1s,3s)-3-((4-(4-(1-(pentan-3-yl)-1H-pyrazol-4-yl)pyrazolo[1 ,5-a]pyrazin-6-yl)-1H-pyrazol-1-yl)methyl)cyclobutanol; cis-4-(4-(4-(1-(pentan-3-yl)-1H-pyrazol-4-yl)pyrazolo[1,5-a]pyrazin-6-yl)-1H-pyrazol-1-yl)cyclohexanol; ((1s,3s)-3-((4-(4-(1-(pentan-3-yl)-1H-pyrazol-4-yl)pyrazolo[1 ,5-a]pyrazin-6-yl)-1H-pyrazol-1-yl)methyl)cyclobutyl)methanol; ((1r,3r)-3-((4-(4-(1-(pentan-3-yl)-1H-pyrazol-4-yl)pyrazolo[1,5-a]pyrazin-6-yl)-1H-pyrazol-1-yl)methyl)cyclobutyl)methanol; 2-methyl-2-(4-(4-(1-(pentan-3-yl)-1H-pyrazol-4-yl)pyrazolo[1 ,5-a]pyrazin-6-yl)-1H-pyrazol-1-yl)propan-1-ol; (S)-2-(4-(4-(1-(pentan-3-yl)-1H-pyrazol-4-yl)pyrazolo[1 ,5-a]pyrazin-6-yl)-1H-pyrazol-1-yl)propan-1-ol; (S)-2-(4-(4-(1-(pentan-3-yl)-1H-pyrazol-4-yl)pyrazolo[1 ,5-a]pyrazin-6-yl)-1H-pyrazol-1-yl)propan-1-ol; (S)-2-((4-(4-(1-(pentan-3-yl)-1H-pyrazol-4-yl)pyrazolo[1 ,5-a]pyrazin-6-yl)-1H-pyrazol-1-yl)methyl)morpholine; (R)-2-((4-(4-(1-(pentan-3-yl)-1H-pyrazol-4-yl)pyrazolo[1,5-a]pyrazin-6-yl)-1H-pyrazol-1-yl)methyl)morpholine; (S)-2-(dimethylamino)-3-(4-(4-(1-(pentan-3-yl)-1H-pyrazol-4-yl)pyrazolo[1 ,5-a]pyrazin-6-yl)-1H-pyrazol-1-yl)propan-1-ol; (R)-2-amino-3-(4-(4-(1-(pentan-3-yl)-1H-pyrazol-4-yl)pyrazolo[1 ,5-a]pyrazin-6-yl)-1H-pyrazol-1-yl)propan-1-ol; (R)-2-(dimethylamino)-3-(4-(4-(1 -(pentan-3-yl)-1H-pyrazol-4-yl)pyrazolo[1,5-a]pyrazin-6-yl)-1H-pyrazol-1-yl)propan-1-ol; (1R,2S,4s)-4-(4-(4-(1-(pentan-3-yl)-1H-pyrazol-4-yl)pyrazolo[1 ,5-a]pyrazi- n-6-yl)-1H-pyrazol-1-yl)cyclopentane-1 ,2-diol; (1R,2S,4r)-4-(4-(4-(1-(pentan-3-yl)-1H-pyrazol-4-yl)pyrazolo[1 ,5-a]pyrazi- n-6-yl)-1H-pyrazol-1-yl)cyclopentane-1 ,2-diol; N-(2-(4-(4-(1-(pentan-3-yl)-1H-pyrazol-4-yl)pyrazolo[1 ,5-a]pyrazin-6-yl)-1H-pyrazol-1-yl)ethyl)cyclopropanamine; 4-(1-(pentan-3-yl)-1H-pyrazol-4-yl)-6-(1-(2-(pyrrolidin-1-yl)ethyl)-1H-py- razol-4-yl)pyrazolo[1 ,5-a]pyrazine; (R)-6-(1-(2-(3-methoxypyrrolidin-1-yl)ethyl)-1H-pyrazol-4-yl)-4-(1-(penta- n-3-yl)-1H-pyrazol-4-yl)pyrazolo[1 ,5-a]pyrazine; (S)-6-(1-(2-(3-methoxypyrrolidin-1-yl)ethyl)-1H-pyrazol-4-yl)-4-(1-(penta- n-3-yl)-1H-pyrazol-4-yl)pyrazolo[1 ,5-a]pyrazine; 6-(1-(2-(3-fluoroazetidin-1-yl)ethyl)-1H-pyrazol-4-yl)-4-(1-(pentan-3-yl)-1 H-pyrazol-4-yl)pyrazolo[1 ,5-a]pyrazine; 1-(2-(4-(4-(1-(pentan-3-yl)-1H-pyrazol-4-yl)pyrazolo[1 ,5-a]pyrazin-6-yl)-1H-pyrazol-1-yl)ethyl)piperidin-4-ol; (R)-1-(2-(4-(4-(1-(pentan-3-yl)-1H-pyrazol-4-yl)pyrazolo[1,5-a]pyrazin-6-- yl)-1H-pyrazol-1-yl)ethyl)pyrrolidin-3-ol; (S)-1-(2-(4-(4-(1-(pentan-3-yl)-1H-pyrazol-4-yl)pyrazolo[1 ,5-a]pyrazin-6-- yl)-1H-pyrazol-1-yl)ethyl)pyrrolidin-3-ol; (S)-1-methyl-3-((2-(4-(4-(1-(pentan-3-yl)-1H-pyrazol-4-yl)pyrazolo[1 ,5-a]- pyrazin-6-yl)-1H-pyrazol-1-yl)ethyl)amino)pyrrolidin-2-one; (R)-1-methyl-3-((2-(4-(4-(1-(pentan-3-yl)-1H-pyrazol-4-yl)pyrazolo[1,5-a]- pyrazin-6-yl)-1H-pyrazol-1-yl)ethyl)amino)pyrrolidin-2-one; 4-(2-(4-(4-(1-(pentan-3-yl)-1H-pyrazol-4-yl)pyrazolo[1,5-a]pyrazin-6-yl)-1H-pyrazol-1-yl)ethyl)piperazin-2-one; (3-((2-(4-(4-(1-(pentan-3-yl)-1H-pyrazol-4-yl)pyrazolo[1 ,5-a]pyrazin-6-yl)-1H-pyrazol-1-yl)ethyl)amino)cyclobutyl)methanol; (1-(2-(4-(4-(1-(pentan-3-yl)-1H-pyrazol-4-yl)pyrazolo[1,5-a]pyrazin-6-yl)-1H-pyrazol-1-yl)ethyl)piperidin-4-yl)methanol; 1-(2-(4-(4-(1-(pentan-3-yl)-1H-pyrazol-4-yl)pyrazolo[1,5-a]pyrazin-6-yl)-1H-pyrazol-1-yl)ethyl)piperazin-2-one; (R)-2-methoxy-3-(4-(4-(1-(pentan-3-yl)-1H-pyrazol-4-yl)pyrazolo[1 ,5-a]pyrazin-6-yl)-1H-pyrazol-1-yl)propan-1-amine; (S)-2-methoxy-3-(4-(4-(1-(pentan-3-yl)-1H-pyrazol-4-yl)pyrazolo[1 ,5-a]pyrazin-6-yl)-1H-pyrazol-1-yl)propan-1-amine; 2-(4-(4-(1-((1R,2R)-2-methylcyclohexyl)-1H-pyrazol-4-yl)pyrazolo[1 ,5-a]pyrazin-6-yl)-1H-pyrazol-1-yl)propane-1 ,3-diol; (R)-3-(4-(4-(1-((1R,2R)-2-methylcyclohexyl)-1H-pyrazol-4-yl)pyrazolo[1 ,5-- a]pyrazin-6-yl)-1H-pyrazol-1-yl)propane-1 ,2-diol; (S)-3-(4-(4-(1-((1R,2R)-2-methylcyclohexyl)-1H-pyrazol-4-yl)pyrazolo[1 ,5-- a]pyrazin-6-yl)-1H-pyrazol-1-yl)propane-1 ,2-diol; (1R,2R)-2-(4-(4-(1-(pentan-3-yl)-1H-pyrazol-4-yl)pyrazolo[1,5-a]pyrazin-6-yl)-1H-pyrazol-1-yl)cyclopentanol; (1S,2S)-2-(4-(4-(1-(pentan-3-yl)-1H-pyrazol-4-yl)pyrazolo[1 ,5-a]pyrazin-6-yl)-1H-pyrazol-1-yl)cyclopentanol; (2S,3S)-3-(4-(4-(1-(pentan-3-yl)-1H-pyrazol-4-yl)pyrazolo[1 ,5-a]pyrazin-6-yl)-1H-pyrazol-1-yl)butan-2-ol; (2R,3R)-3-(4-(4-(1-(pentan-3-yl)-1H-pyrazol-4-yl)pyrazolo[1 ,5-a]pyrazin-6-yl)-1H-pyrazol-1-yl)butan-2-ol; (2R,3S)-3-(4-(4-(1-(pentan-3-yl)-1H-pyrazol-4-yl)pyrazolo[1 ,5-a]pyrazin-6-yl)-1H-pyrazol-1-yl)butan-2-ol; (2S,3R)-3-(4-(4-(1-(pentan-3-yl)-1H-pyrazol-4-yl)pyrazolo[1 ,5-a]pyrazin-6-yl)-1H-pyrazol-1-yl)butan-2-ol; (3S,4R)-4-(4-(4-(1-(pentan-3-yl)-1H-pyrazol-4-yl)pyrazolo[1,5-a]pyrazin-6-yl)-1H-pyrazol-1-yl)tetrahydrofuran-3-ol; (3R,4S)-4-(4-(4-(1-(pentan-3-yl)-1H-pyrazol-4-yl)pyrazolo[1,5-a]pyrazin-6-yl)-1H-pyrazol-1-yl)tetrahydrofuran-3-ol; trans-3-(4-(4-(1-(pentan-3-yl)-1H-pyrazol-4-yl)pyrazolo[1 ,5-a]pyrazin-6-yl)-1H-pyrazol-1-yl)cyclobutanol; (1s,3s)-1-methyl-3-(4-(4-(1-(pentan-3-yl)-1H-pyrazol-4-yl)pyrazolo[1 ,5-a]- pyrazin-6-yl)-1H-pyrazol-1-yl)cyclobutanol; (1s,3s)-1 -(hydroxymethyl)-3-(4-(4-(1 -(pentan-3-yl)-1H-pyrazol-4-yl)pyrazolo[1 ,5-a]pyrazin-6-yl)-1H-pyrazol-1-yl)cyclobutanol; (1r,3r)-1-(hydroxymethyl)-3-(4-(4-(1-(pentan-3-yl)-1H-pyrazol-4-yl)pyrazolo[1 ,5-a]pyrazin-6-yl)-1H-pyrazol-1-yl)cyclobutanol; (trans-3-(4-(4-(1-(pentan-3-yl)-1H-pyrazol-4-yl)pyrazolo[1 ,5-a]pyrazin-6-- yl)-1H-pyrazol-1-yl)cyclobutyl)methanol; (cis-3-(4-(4-(1-(pentan-3-yl)-1H-pyrazol-4-yl)pyrazolo[1 ,5-a]pyrazin-6-yl)-1H-pyrazol-1-yl)cyclobutyl)methanol; (1r,3r)-3-(4-(4-(1-(pentan-3-yl)-1H-pyrazol-4-yl)pyrazolo[1 ,5-a]pyrazin-6-yl)-1H-pyrazol-1-yl)cyclobutanecarboxamide; (1s,3s)-3-(4-(4-(1-(pentan-3-yl)-1H-pyrazol-4-yl)pyrazolo[1,5-a]pyrazin-6-yl)-1H-pyrazol-1-yl)cyclobutanecarboxamide; (1r,3r)-N,N-dimethyl-3-(4-(4-(1-(pentan-3-yl)-1H-pyrazol-4-yl)pyrazolo[1,- 5-a]pyrazin-6-yl)-1H-pyrazol-1-yl)cyclobutanecarboxamide; (1s,3s)-N,N-dimethyl-3-(4-(4-(1-(pentan-3-yl)-1H-pyrazol-4-yl)pyrazolo[1,- 5-a]pyrazin-6-yl)-1H-pyrazol-1-yl)cyclobutanecarboxamide; (1r,3r)-N-(2-hydroxyethyl)-3-(4-(4-(1-(pentan-3-yl)-1H-pyrazol-4-yl)pyraz- olo[1 ,5-a]pyrazin-6-yl)-1H-pyrazol-1-yl)cyclobutanecarboxamide; (1s,3s)-N-(2-hydroxyethyl)-3-(4-(4-(1-(pentan-3-yl)-1H-pyrazol-4-yl)pyraz- olo[1,5-a]pyrazin-6-yl)-1H-pyrazol-1-yl)cyclobutanecarboxamide; (S)-2-hydroxy-1-(3-(4-(4-(1-(pentan-3-yl)-1H-pyrazol-4-yl)pyrazolo[1 ,5-a]- pyrazin-6-yl)-1H-pyrazol-1-yl)azetidin-1-yl)propan-1-one; 2-hydroxy-1-(3-(4-(4-(1-(pentan-3-yl)-1H-pyrazol-4-yl)pyrazolo[1,5-a]pyrazin-6-yl)-1H-pyrazol-1-yl)azetidin-1-yl)ethanone; (R)-2-hydroxy-1-(3-(4-(4-(1-(pentan-3-yl)-1H-pyrazol-4-yl)pyrazolo[1 ,5-a]- pyrazin-6-yl)-1H-pyrazol-1-yl)azetidin-1-yl)propan-1-one; 2-hydroxy-2-methyl-1-(3-(4-(4-(1-(pentan-3-yl)-1H-pyrazol-4-yl)pyrazolo[1 ,5-a]pyrazin-6-yl)-1H-pyrazol-1-yl)azetidin-1-yl)propan-1-one; (1-hydroxycyclopropyl)(3-(4-(4-(1-(pentan-3-yl)-1H-pyrazol-4-yl)pyrazolo[- 1,5-a]pyrazin-6-yl)-1H-pyrazol-1-yl)azetidin-1-yl)methanone; (cis-3-hydroxycyclobutyl)(3-(4-(4-(1-(pentan-3-yl)-1H-pyrazol-4-yl)pyrazolo[1,5-a]pyrazin-6-yl)-1H-pyrazol-1-yl)azetidin-1-yl)methanone; (trans-3-hydroxycyclobutyl)(3-(4-(4-(1-(pentan-3-yl)-1H-pyrazol-4-yl)pyra- zolo[1 ,5-a]pyrazin-6-yl)-1H-pyrazol-1-yl)azetidin-1-yl)methanone; 2-(3-hydroxyazetidin-1-yl)-1-(3-(4-(4-(1-(pentan-3-yl)-1H-pyrazol-4-yl)py- razolo[1 ,5-a]pyrazin-6-yl)-1H-pyrazol-1-yl)azetidin-1-yl)ethanone; 1-(3-(4-(4-(1-(pentan-3-yl)-1H-pyrazol-4-yl)pyrazolo[1 ,5-a]pyrazin-6-yl)-1H-pyrazol-1-yl)azetidin-1-yl)-2-(pyrrolidin-1-yl)ethanone; N,N-dimethyl-2-oxo-2-(3-(4-(4-(1-(pentan-3-yl)-1H-pyrazol-4-yl)pyrazolo[1 ,5-a]pyrazin-6-yl)-1H-pyrazol-1-yl)azetidin-1-yl)acetamide; (S)-3-amino-2-methyl-4-(4-(4-(1-(pentan-3-yl)-1H-pyrazol-4-yl)pyrazolo[1,- 5-a]pyrazin-6-yl)-1H-pyrazol-1-yl)butan-2-ol; 4-((4-(4-(1-(pentan-3-yl)-1H-pyrazol-4-yl)pyrazolo[1,5-a]pyrazin-6-yl)-1H- -pyrazol-1-yl)methyl)piperidin-4-ol; 1-(4-hydroxy-4-((4-(4-(1-(pentan-3-yl)-1H-pyrazol-4-yl)pyrazolo[1 ,5-a]pyrazin-6-yl)-1H-pyrazol-1-yl)methyl)piperidin-1-yl)ethanone; 2-amino-1-(3-(4-(4-(1-(pentan-3-yl)-1H-pyrazol-4-yl)pyrazolo[1 ,5-a]pyrazi- n-6-yl)-1H-pyrazol-1-yl)azetidin-1-yl)ethanone; 2-(methylamino)-1-(3-(4-(4-(1-(pentan-3-yl)-1H-pyrazol-4-yl)pyrazolo[1 ,5-- a]pyrazin-6-yl)-1H-pyrazol-1-yl)azetidin-1-yl)ethanone; (3R,4R)-4-(4-(4-(1-(pentan-3-yl)-1H-pyrazol-4-yl)pyrazolo[1 ,5-a]pyrazin-6-yl)-1H-pyrazol-1-yI)piperidin-3-ol; (1S,2R)-2-(4-(4-(1-(pentan-3-yl)-1H-pyrazol-4-yl)pyrazolo[1,5-a]pyrazin-6-yl)-1H-pyrazol-1-yl)cyclopentanol; (1R,2S)-2-(4-(4-(1-(pentan-3-yl)-1H-pyrazol-4-yl)pyrazolo[1,5-a]pyrazin-6-yl)-1H-pyrazol-1-yl)cyclopentanol; 2,2-dimethyl-3-(4-(4-(1-(pentan-3-yl)-1H-pyrazol-4-yl)pyrazolo[1 ,5-a]pyrazin-6-yl)-1H-pyrazol-1-yl)propan-1-ol; (1-((4-(4-(1-(pentan-3-yl)-1H-pyrazol-4-yl)pyrazolo[1,5-a]pyrazin-6-yl)-1-H-pyrazol-1-yl)methyl)cyclopropyl)methanol; (2R)-3-(4-(4-(1-(1,1,1-trifluorobutan-2-yl)-1H-pyrazol-4-yl)pyrazolo[1 ,5-- a]pyrazin-6-yl)-1H-pyrazol-1-yl)propane-1 ,2-diol; (R)-3-(4-(4-(1-((R)-4,4,4-trifluorobutan-2-yl)-1H-pyrazol-4-yl)pyrazolo[1,5-a]pyrazin-6-yl)-1H-pyrazol-1-yl)propane-1,2-diol; (R)-3-(4-(4-(1-((S)-4,4,4-trifluorobutan-2-yl)-1H-pyrazol-4-yl)pyrazolo[1,5-a]pyrazin-6-yl)-1H-pyrazol-1-yl)propane-1,2-diol; (R)-3-(4-(4-(1-((R)-1-cyclobutylpropyl)-1H-pyrazol-4-yl)pyrazolo[1 ,5-a]pyrazin-6-yl)-1H-pyrazol-1-yl)propane-1 ,2-diol; (R)-3-(4-(4-(1-((S)-1-cyclobutylpropyl)-1H-pyrazol-4-yl)pyrazolo[1 ,5-a]pyrazin-6-yl)-1H-pyrazol-1-yl)propane-1 ,2-diol; (R)-3-(4-(4-(1-((S)-2,2-difluoropentan-3-yl)-1H-pyrazol-4-yl)pyrazolo[1 ,5-a]pyrazin-6-yl)-1H-pyrazol-1-yl)propane-1 ,2-diol; (R)-3-(4-(4-(-((R)-2,2-difluoropentan-3-yl)-1H-pyrazol-4-yl)pyrazolo[1 ,5-a]pyrazin-6-yl)-1H-pyrazol-1-yl)propane-1 ,2-diol (S)-4-(4-(4-(1-(pentan-3-yl)-1H-pyrazol-4-yl)pyrazolo[1 ,5-a]pyrazin-6-yl)-1H-pyrazol-1-yl)butane-1,3-diol; (R)-4-(4-(4-(1-(pentan-3-yl)-1H-pyrazol-4-yl)pyrazolo[1 ,5-a]pyrazin-6-yl)-1H-pyrazol-1-yl)butane-1,3-diol; (R)-2-methyl-3-(4-(4-(1-(pentan-3-yl)-1H-pyrazol-4-yl)pyrazolo[1,5-a]pyrazin-6-yl)-1H-pyrazol-1-yl)propane-1 ,2-diol; (S)-2-methyl-3-(4-(4-(1-(pentan-3-yl)-1H-pyrazol-4-yl)pyrazolo[1 ,5-a]pyrazin-6-yl)-1H-pyrazol-1-yl)propane-1,2-diol; (R)-3-(4-(4-(1-((R)-1-((S)-2,2-difluorocyclopropyl)propyl)-1H-pyrazol-4-y-l)pyrazolo[1 ,5-a]pyrazin-6-yl)-1H-pyrazol-1-yl)propane-1 ,2-diol; (R)-3-(4-(4-(1-((S)-1-cyclopropylbutan-2-yl)-1H-pyrazol-4-yl)pyrazolo[1 ,5-a]pyrazin-6-yl)-1H-pyrazol-1-yl)propane-1,2-diol; (R)-3-(4-(4-(1-((R)-1-cyclopropylbutan-2-yl)-1H-pyrazol-4-yl)pyrazolo[1,5-a]pyrazin-6-yl)-1H-pyrazol-1-yl)propane-1,2-diol; (R)-3-(4-(4-(1-((1S,2R)-2-ethylcyclopentyl)-1H-pyrazol-4-yl)pyrazolo[1 ,5-- a]pyrazin-6-yl)-1H-pyrazol-1-yl)propane-1 ,2-diol; (R)-3-(4-(4-(1-((1R,2S)-2-ethylcyclopentyl)-1H-pyrazol-4-yl)pyrazolo[1 ,5--a]pyrazin-6-yl)-1H-pyrazol-1-yl)propane-1,2-diol; (R)-3-(4-(4-(1-((R)-1,1,1-trifluoropentan-3-yl)-1H-pyrazol-4-yl)pyrazolo[- 1 ,5-a]pyrazin-6-yl)-1H-pyrazol-1-yl)propane-1 ,2-diol; (R)-3-(4-(4-(1-((S)-1,1,1-trifluoropentan-3-yl)-1H-pyrazol-4-yl)pyrazolo[-1,5-a]pyrazin-6-yl)-1H-pyrazol-1-yl)propane-1 ,2-diol; (R)-2-(4-(4-(1-(1,1,1-trifluoropentan-3-yl)-1H-pyrazol-4-yl)pyrazolo[1 ,5--a]pyrazin-6-yl)-1H-pyrazol-1-yl)propane-1,3-diol; (S)-2-(4-(4-(1-(1,1,1-trifluoropentan-3-yl)-1H-pyrazol-4-yl)pyrazolo[1 ,5-- a]pyrazin-6-yl)-1H-pyrazol-1-yl)propane-1 ,3-diol; (R)-3-(4-(4-(1-((R)-1-cyclopropylpropyl)-1H-pyrazol-4-yl)pyrazolo[1 ,5-a]pyrazin-6-yl)-1H-pyrazol-1-yl)propane-1 ,2-diol; (R)-3-(4-(4-(1-((S)-1-cyclopropylpropyl)-1H-pyrazol-4-yl)pyrazolo[1,5-a]pyrazin-6-yl)-1H-pyrazol-1-yl)propane-1 ,2-diol; (R)-2-(4-(4-(1-(1-cyclopropylpropyl)-1H-pyrazol-4-yl)pyrazolo[1 ,5-a]pyrazin-6-yl)-1H-pyrazol-1-yl)propane-1 ,3-diol; (R)-3-(4-(4-(1-(dicyclopropylmethyl)-1H-pyrazol-4-yl)pyrazolo[1 ,5-a]pyrazin-6-yl)-1H-pyrazol-1-yl)propane-1 ,2-diol; (R)-3-(4-(4-(1-(cis-2-methylcyclobutyl)-1H-pyrazol-4-yl)pyrazolo[1 ,5-a]pyrazin-6-yl)-1H-pyrazol-1-yl)propane-1 ,2-diol; and (S)-1 -(4-(4-(1-((R)-1-cyclopropylpropyl)-1H-pyrazol-4-yl)pyrazolo[1,5-a]pyrazin-6-yl)-1H-pyrazol-1-yl)propan-2-ol; or a pharmaceutically acceptable salt thereof.

In another embodiment, the present invention provides a method of treating a patient infected with SARS-CoV-2 comprising administering to the patient in need of such treatment a therapeutically effective amount (R)-3-(4-(4-(1-(pentan-3-yl)-1H-pyrazol-4-yl)pyrazolo[1,5-a]pyrazin-6-yl)-1H-pyrazol-1-yl)propane-1 ,2-diol or a pharmaceutically acceptable salt thereof.

The present invention is related to methods for treating COVID-19 disease in a patient, the method comprising administering to the patient in need of such treatment compounds which inhibit IRAK4. The present invention includes the IRAK4 inhibitors described in WO2015/150995, herein incorporated by reference in its entirety. Accordingly, in another embodiment, the present invention provides a method of treating a patient infected with SARS-CoV-2 comprising administering to the patient in need of such treatment a therapeutically effective amount of a compound selected from the group consisting of:
4-(azetidin-3-ylmethoxy)-6-(propan-2-yloxy)quinoline-7-carboxamide;
4-[(3S)-piperidin-3-ylmethoxy]-6-(propan-2-yloxy)quinoline-7-carboxamide;
4-[(3R)-piperidin-3-ylmethoxy]-6-(propan-2-yloxy)quinoline-7-carboxamide;
4-(piperidin-4-ylmethoxy)-6-(propan-2-yloxy)quinoline-7-carboxamide;
4-[(1R,5S,6r)-3-azabicyclo[3.1 .0]hex-6-ylmethoxy]-6-(propan-2-yloxy)quinoline-7-carboxamide;
4-(oxetan-3-ylmethoxy)-6-(propan-2-yloxy)quinoline-7-carboxamide;
4-(cyclopentylmethoxy)-6-(propan-2-yloxy)quinoline-7-carboxamide;
4-(1-cyclobutylethoxy)-6-(propan-2-yloxy)quinoline-7-carboxamide;
4-(cyclobutylmethoxy)-6-(propan-2-yloxy)quinoline-7-carboxamide;
6-(propan-2-yloxy)-4-(tetrahydrofuran-3-ylmethoxy)quinoline-7-carboxamide;
6-(propan-2-yloxy)-4-(tetrahydrofuran-2-ylmethoxy)quinoline-7-carboxamide;
4-[(3-methyloxetan-3-yl)methoxy]-6-(propan-2-yloxy)quinoline-7-carboxamide;
4-[(1-methylcyclobutyl)methoxy]-6-(propan-2-yloxy)quinoline-7-carboxamide;
4-[(2R)-bicyclo[2.2.1]hept-2-yloxy]-6-(propan-2-yloxy)quinoline-7-carboxamide;
6-(propan-2-yloxy)-4-[(2R)-tetrahydrofuran-2-ylmethoxy]quinoline-7-carboxamide;
4-(bicyclo[2.2.1]hept-2-yloxy)-6-(propan-2-yloxy)quinoline-7-carboxamide;
6-(propan-2-yloxy)-4-(tricyclo[2.2.1.0-2,6-jhept-3-yloxy)quinoline-7-carboxamide;
4-(1,3-dioxolan-4-ylmethoxy)-6-(propan-2-yloxy)quinoline-7-carboxamide;
4-[(1S,2R)-bicyclo[2.2.1]hept-2-yloxy]-6-(propan-2-yloxy)quinoline-7-carboxamide;
1-[(3aR,6aS)-octahydrocyclopenta[c]pyrrol-4-yloxy]-7-(propan-2-yloxy)isoquinoline-6-carboxamide;
4-[(3aR,6aS)-octahydrocyclopenta[c]pyrrol-4-yloxy]-6-(propan-2-yloxy)quinoline-7-carboxamide;
4-{[(3S)-1-(cyanoacetyl)pyrrolidin-3-yl]methoxy}-6-(propan-2-yloxy)quinoline-7-carboxamide;
4-{[(3R)-1-(cyanoacetyl)pyrrolidin-3-yl]methoxy}-6-(propan-2-yloxy)quinoline-7-carboxamide;
7-(propan-2-yloxy)-1-(tetrahydrofuran-3-ylmethoxy)isoquinoline-6-carboxamide;
7-(propan-2-yloxy)-1-(tetrahydro-2H-pyran-2-ylmethoxy)isoquinoline-6-carboxamide;
1-{[(2S)-5-oxopyrrolidin-2-yl] methoxy}-7-(propan-2-yloxy)isoquinoline-6-carboxamide;
1-[(1,1-dioxido-1,2-thiazinan-3-yl)methoxy]-7-(propan-2-yloxy)isoquinoline-6-carboxamide;
1-[(3S)-piperidin-3-ylmethoxy]-7-(propan-2-yloxy)isoquinoline-6-carboxamide;
1-[(3-methyl-2-oxo-1,3-oxazolidin-4-yl)methoxy]-7-(propan-2-yloxy)isoquinoline-6-carboxamide;
7-(propan-2-yloxy)-1-[(2R)-tetrahydrofuran-2-ylmethoxy]isoquinoline-6-carboxamide;
1-{[(2S)-1-methylpyrrolidin-2-yl]methoxy}-7-(propan-2-yloxy)isoquinoline-6-carboxamide;
1-{[(2R)-5-oxopyrrolidin-2-yl]methoxy}-7-(propan-2-yloxy)isoquinoline-6-carboxamide;
1-[(1-acetylpiperidin-4-yl)methoxy]-7-(propan-2-yloxy)isoquinoline-6-carboxamide;
1-{[(3R,4R)-4-methoxypyrrolidin-3-yl]methoxy}-7-(propan-2-yloxy)isoquinoline-6-carboxamide;
1-[(2-oxo-1,3-oxazolidin-5-yl)methoxy]-7-(propan-2-yloxy)isoquinoline-6-carboxamide;
7-(propan-2-yloxy)-1-(tetrahydro-2H-pyran-4-ylmethoxy)isoquinoline-6-carboxamide;
1-[(2S)-morpholin-2-ylmethoxy]-7-(propan-2-yloxy)isoquinoline-6-carboxamide;
1-[(4-fluoropiperidin-4-yl)methoxy]-7-(propan-2-yloxy)isoquinoline-6-carboxamide;
1-(morpholin-2-ylmethoxy)-7-(propan-2-yloxy)isoquinoline-6-carboxamide;
1-[(1S,5S)-3-azabicyclo[3.1.0]hex-1-ylmethoxy]-7-(propan-2-yloxy)isoquinoline-6-carboxamide;
7-(propan-2-yloxy)-1-[(2R)-pyrrolidin-2-ylmethoxy]isoquinoline-6-carboxamide;
1-[(1R,5S,6r)-3-azabicyclo[3.1.0]hex-6-ylmethoxy]-7-(propan-2-yloxy)isoquinoline-6-carboxamide;
1-(piperidin-2-ylmethoxy)-7-(propan-2-yloxy)isoquinoline-6-carboxamide;
1-[(4-methylmorpholin-2-yl)methoxy]-7-(propan-2-yloxy)isoquinoline-6-carboxamide;
1-[(1-methylpiperidin-3-yl)methoxy]-7-(propan-2-yloxy)isoquinoline-6-carboxamide;
7-(propan-2-yloxy)-1-{[(3R,4R)-4-(trifluoromethyl)pyrrolidin-3-yl]methoxy}isoquinoline-6-carboxamide;
1-[(2R)-morpholin-2-ylmethoxy]-7-(propan-2-yloxy)isoquinoline-6-carboxamide;
1-[(3R)-piperidin-3-ylmethoxy]-7-(propan-2-yloxy)isoquinoline-6-carboxamide;
7-(propan-2-yloxy)-1-[(3S)-pyrrolidin-3-ylmethoxy]isoquinoline-6-carboxamide;
6-(propan-2-yloxy)-4-[(3S)-pyrrolidin-3-ylmethoxy]quinoline-7-carboxamide;
4-[(2S)-morpholin-2-ylmethoxy]-6-(propan-2-yloxy)quinoline-7-carboxamide;
4-(7-azaspiro[3.5]non-1-yloxy)-6-(propan-2-yloxy)quinoline-7-carboxamide;
4-[(2R)-morpholin-2-ylmethoxy]-6-(propan-2-yloxy)quinoline-7-carboxamide;
4-[(4-fluoropiperidin-4-yl)methoxy]-6-(propan-2-yloxy)quinoline-7-carboxamide;
4-{[(3R,4R)-3,4-dimethylpyrrolidin-3-yl]methoxy}-6-(propan-2-yloxy)quinoline-7-carboxamide;
4-[(4-methylpiperidin-4-yl)methoxy]-6-(propan-2-yloxy)quinoline-7-carboxamide;
4-{[(5R)-2-oxo-1,3-oxazolidin-5-yl]methoxy}-6-(propan-2-yloxy)quinoline-7-carboxamide;
4-[(3-methylpiperidin-3-yl)methoxy]-6-(propan-2-yloxy)quinoline-7-carboxamide;
4-(piperidin-3-ylmethoxy)-6-(propan-2-yloxy)quinoline-7-carboxamide;
4-{[1-(cyanoacetyl)azetidin-3-yl]methoxy}-6-(propan-2-yloxy)quinoline-7-carboxamide;
1-{[(2R)-1-(cyanoacetyl)pyrrolidin-2-yl]methoxy}-7-(propan-2-yloxy)isoquinoline-6-carboxamide;
1-{[1-(cyanoacetyl)piperidin-4-yl]methoxy}-7-(propan-2-yloxy)isoquinoline-6-carboxamide;
1-{[(2S)-1-(cyanoacetyl)pyrrolidin-2-yl]methoxy}-7-(propan-2-yloxy)isoquinoline-6-carboxamide;
1-{[(3R)-4-(cyanoacetyl)morpholin-3-yl]methoxy}-7-(propan-2-yloxy)isoquinoline-6-carboxamide;
1-({1-[(cyanoacetyl)amino]cyclopentyl}methoxy)-7-(propan-2-yloxy)isoquinoline-6-carboxamide;
1-{[(3S)-1-(cyanoacetyl)pyrrolidin-3-yl]methoxy}-7-(propan-2-yloxy)isoquinoline-6-carboxamide;
1-{[(3R)-1-(cyanoacetyl)piperidin-3-yl]methoxy}-7-(propan-2-yloxy)isoquinoline-6-carboxamide;
1-{[(1R,5R,6R)-3-(cyanoacetyl)-3-azabicyclo[3.2.1]oct-6-yl]oxy}-7-(propan-2-yloxy)isoquinoline-6-carboxamide;
1-{[1-(cyanoacetyl)azetidin-3-yl]methoxy}-7-(propan-2-yloxy)isoquinoline-6-carboxamide;
1-{[(3R)-1-(cyanoacetyl)pyrrolidin-3-yl]methoxy}-7-(propan-2-yloxy)isoquinoline-6-carboxamide;
4-{[(3aR,6aS)-2-(cyanoacetyl)octahydrocyclopenta[c]pyrrol-4-yl]oxy}-6-(propan-2-yloxy)quinoline-7-carboxamide;
1-[(1S,4R)-2-azabicyclo[2.2.1]hept-6-yloxy]-7-(propan-2-yloxy)isoquinoline-6-carboxamide;
1-{[(2S)-1-(cyanoacetyl)azetidin-2-yl]methoxy}-7-(propan-2-yloxy)isoquinoline-6-carboxamide;
1-{[(1S,4S,5S)-2-(cyanoacetyl)-2-azabicyclo[2.2.1]hept-5-yl]oxy}-7-(propan-2-yloxy)isoquinoline-6-carboxamide;
1-{[(2S)-4-(cyanoacetyl)morpholin-2-yl]methoxy}-7-(propan-2-yloxy)isoquinoline-6-carboxamide;
1-{[1-(cyanoacetyl)-4-fluoropiperidin-4-yl]methoxy}-7-(propan-2-yloxy)isoquinoline-6-carboxamide;
1-{[(1S,5S)-3-(cyanoacetyl)-3-azabicyclo[3.1.0]hex-1-yl]methoxy}-7-(propan-2-yloxy)isoquinoline-6-carboxamide;
1-{[(2R)-4-(cyanoacetyl)morpholin-2-yl]methoxy}-7-(propan-2-yloxy)isoquinoline-6-carboxamide;
1-{[(3aR,4S,6aS)-2-(cyanoacetyl)octahydrocyclopenta[c]pyrrol-4-yl]oxy}-7-(propan-2-yloxy)isoquinoline-6-carboxamide;
1-{[(3R,4R)-1-(cyanoacetyl)-4-ethylpyrrolidin-3-yl]methoxy}-7-(propan-2-yloxy)isoquinoline-6-carboxamide;
1-{[(1S,5S,6S)-3-(cyanoacetyl)-3-azabicyclo[3.2.1]oct-6-yl]oxy}-7-(propan-2-yloxy)isoquinoline-6-carboxamide;
1-{[1-(cyanoacetyl)-3-methylpyrrolidin-3-yl]methoxy}-7-(propan-2-yloxy)isoquinoline-6-carboxamide;
1-{[(3S)-4-(cyanoacetyl)morpholin-3-yl]methoxy}-7-(propan-2-yloxy)isoquinoline-6-carboxamide;
1-{[(3R,4R)-1-(cyanoacetyl)-4-methoxypyrrolidin-3-yl]methoxy}-7-(propan-2-yloxy)isoquinoline-6-carboxamide;
1-{[(3R,4R)-1-(cyanoacetyl)-4-methylpyrrolidin-3-yl]methoxy}-7-(propan-2-yloxy)isoquinoline-6-carboxamide;
1-{[1-(cyanoacetyl)-4-methylpiperidin-4-yl]methoxy}-7-(propan-2-yloxy)isoquinoline-6-carboxamide;
4-{[(1R,5S,6r)-3-(cyanoacetyl)-3-azabicyclo[3.1,0]hex-6-yl]methoxy}-6-(propan-2-yloxy)quinoline-7-carboxamide;
4-{[(3R,4R)-1-(cyanoacetyl)-4-methylpyrrolidin-3-yl]methoxy}-6-(propan-2-yloxy)quinoline-7-carboxamide;
4-{[(1R,5R6R)-3-(cyanoacetyl)-3-azabicyclo[3.2.1]oct-6-yl]oxy}-6-(propan-2-yloxy)quinoline-7-carboxamide;
4-{[(1S,5S)-3-(cyanoacetyl)-3-azabicyclo[3.1.0]hex-1-yl]methoxy}-6-(propan-2-yloxy)quinoline-7-carboxamide;
4-{[1-(cyanoacetyl)-4-methylpiperidin-4-yl]methoxy}-6-(propan-2-yloxy)quinoline-7-carboxamide;
4-{[(1S,5S,6S)-3-(cyanoacetyl)-3-azabicyclo[3.2.1]oct-6-yl]oxy}-6-(propan-2-yloxy)quinoline-7-carboxamide;
4-{[(3S)-1-(cyanoacetyl)piperidin-3-yljmethoxy}-6-(propan-2-yloxy)quinoline-7-carboxamide;
4-{[(1S,5S)-3-(cyanoacetyl)-3-azabicyclo[3.1.0]hex-1-yl]methoxy}-6-(propan-2-yloxy)quinoline-7-carboxamide;
4-{[1-(cyanoacetyl)-4-fluoropiperidin-4-yl]methoxy}-6-(propan-2-yloxy)quinoline-7-carboxamide;
4-{[(3R,4R)-1-(cyanoacetyl)-4-methoxypyrrolidin-3-yl]methoxy}-6-(propan-2-yloxy)quinoline-7-carboxamide;
4-{[(3R)-1-(cyanoacetyl)piperidin-3-yl]methoxy}-6-(propan-2-yloxy)quinoline-7-carboxamide;
4-{[(2S)-4-(cyanoacetyl)morpholin-2-yl]methoxy}-6-(propan-2-yloxy)quinoline-7-carboxamide;
4-{[1-(cyanoacetyl)piperidin-2-yl]methoxy}-6-(propan-2-yloxy)quinoline-7-carboxamide;
4-{[1-(cyanoacetyl)piperidin-4-yl]methoxy}-6-(propan-2-yloxy)quinoline-7-carboxamide;
1-[(1S,4S,5S)-2-azabicyclo[2.2.1]hept-5-yloxy]-7-(propan-2-yloxy)isoquinoline-6-carboxamide;
1-[(1R,4R,5R)-2-azabicyclo[2.2.1]hept-5-yloxy]-7-(propan-2-yloxy)isoquinoline-6-carboxamide;
1-{[(2S)-5-oxopyrrolidin-2-yl] methoxy}-7-(propan-2-yloxy)isoquinoline-6-carbonitrile
1-{[(4R)-2-oxo-1,3-oxazolidin-4-yl]methoxy}-7-(propan-2-yloxy)isoquinoline-6-carboxamide;
4-methyl-1-{[(2S)-5-oxopyrrolidin-2-yl]methoxy}-7-(propan-2-yloxy)isoquinoline-6-carboxamide;
1-{[(2S)-6-oxopiperidin-2-yl]methoxy}-7-(propan-2-yloxy)isoquinoline-6-carboxamide;
1-{[(2S)-4-methyl-5-oxopyrrolidin-2-yl]methooy}-7-(propan-2-yloxy)isoquinoline-6-carboxamide;
1-[(1S,4R,6R)-2-azabicyclo[2.2.1]hept-6-yloxy]-7-(propan-2-yloxy)isoquinoline-6-carboxamide;
1-[(1S,4R,6S)-2-azabicyclo[2.2.1]hept-6-yloxy]-7-(propan-2-yloxy)isoquinoline-6-carboxamide;
1-{[(2S)-4,4-dimethyl-5-oxopyrrolidin-2-yl]methoxy}-7-(propan-2-yloxy)isoquinoline-6-carboxamide;
1-[(5-oxopyrrolidin-3-yl)methoxy]-7-(propan-2-yloxy)isoquinoline-6-carboxamide;
1-{[(2S)-4-fluoro-5-oxopyrrolidin-2-yl]methoxy}-7-(propan-2-yloxy)isoquinoline-6-carboxamide;
1-{[(2S)-4-ethyl-5-oxopyrrolidin-2-yl] methoxy}-7-(propan-2-yloxy)isoquinoline-6-carboxamide;
5-{[(2S)-5-oxopyrrolidin-2-yl]methoxy}-3-(propan-2-yloxy)naphthalene-2-carboxamide;
3-(propan-2-yloxy)-5-[(3R)-pyrrolidin-3-ylmethoxy]naphthalene-2-carboxamide;
1-[(5-oxomorpholin-3-yl)methoxy]-7-(propan-2-yloxy)isoquinoline-6-carboxamide;
7-(propan-2-yloxy)-1-[(3R)-pyrrolidin-3-ylmethoxy]isoquinoline-6-carboxamide;
1-[(3-oxooctahydro-1H-isoindol-1 -yl)methoxy]-7-(propan-2-yloxy)isoquinoline-6-carboxamide;
1-[(2S)-azetidin-2-ylmethoxy]-7-(propan-2-yloxy)isoquinoline-6-carboxamide;
7-(cyclobutyloxy)-1-{[(2S)-5-oxopyrrolidin-2-yl]methoxy}isoquinoline-6-carboxamide;
7-methoxy-1-{[(2S)-5-oxopyrrolidin-2-yl]methoxy}isoquinoline-6-carboxamide;
7-ethoxy-1-{[(2S)-5-oxopyrrolidin-2-yl]methoxy}isoquinoline-6-carboxamide;
1-[(3aR,6aR)-hexahydrocyclopenta[c]pyrrol-3a(1H)-ylmethoxy]-7-(propan-2-yloxy)isoquinoline-6-carboxamide;
1-{[(2S4R)-4-methyl-5-oxopyrrolidin-2-yl]methoxy}-7-(propan-2-yloxy)isoquinoline-6-carboxamide;
1-{[(2S,4S)-4-methyl-5-oxopyrrolidin-2-yl]methoxy}-7-(propan-2-yloxy)isoquinoline-6-carboxamide;
1-{[(2S)-2-methyl-5-oxopyrrolidin-2-yl]methoxy}-7-(propan-2-yloxy)isoquinoline-6-carboxamide;
1-{[(2S)-4-(methoxymethyl)-5-oxopyrrolidin-2-yl]methoxy}-7-(propan-2-yloxy)isoquinoline-6-carboxamide;
1-{[(2S)-4,4-difluoro-5-oxopyrrolidin-2-yl]methoxy}-7-(propan-2-yloxy)isoquinoline-6-carboxamide;
7-(difluoromethoxy)-1-{[(2S)-5-oxopyrrolidin-2-yl]methoxy}isoquinoline-6-carboxamide;
1-{[(3aS,6R,6aR)-2-oxooctahydrocyclopenta[b]pyrrol-6-yl]oxy}-7-(propan-2-yloxy)isoquinoline-6-carboxamide;
1-{[(2S,4S)-4-ethyl-4-fluoro-5-oxopyrrolidin-2-yl]methoxy}-7-(propan-2-yloxy)isoquinoline-6-carboxamide;
1-{[(2S,4R)-4-ethyl-4-fluoro-5-oxopyrrolidin-2-yl]methoxy}-7-(propan-2-yloxy)isoquinoline-6-carboxamide;
1-{[(2S,4R)-4-fluoro-4-methyl-5-oxopyrrolidin-2-yl]methoxy}-7-(propan-2-yloxy)isoquinoline-6-carboxamide;
1-{[(2S,4S)-4-fluoro-4-methyl-5-oxopyrrolidin-2-yl]methoxy}-7-(propan-2-yloxy)isoquinoline-6-carboxamide;
1-{[(2S,4S)-4-ethyl-5-oxopyrrolidin-2-yl]methoxy}-7-(propan-2-yloxy)isoquinoline-6-carboxamide;
1-{[(2S,4R)-4-ethyl-5-oxopyrrolidin-2-yl]methoxy}-7-(propan-2-yloxy)isoquinoline-6-carboxamide;
1-{[(2S,4R)-4-(2-hydroxypropan-2-yl)-5-oxopyrrolidin-2-yl]methoxy}-7-(propan-2-yloxy)isoquinoline-6-carboxamide;
1-[(2-oxopiperidin-4-yl)methoxy]-7-(propan-2-yloxy)isoquinoline-6-carboxamide;
1-[(1S5S)-3-azabicyclo[3.1.0]hex-1-ylmethoxy]-7-(propan-2-yloxy)isoquinoline-6-carboxamide;
1-[(1R,5R)-3-azabicyclo[3.1.0]hex-1-ylmethoxy]-7-(propan-2-yloxy)isoquinoline-6-carboxamide;
1-{[(2S,3S)-3-methyl-5-oxopyrrolidin-2-yl]methoxy}-7-(propan-2-yloxy)isoquinoline-6-carboxamide;
1-[(6-oxopiperidin-3-yl)methoxy]-7-(propan-2-yloxy)isoquinoline-6-carboxamide;
1-[(1,1-dioxido-1,2-thiazolidin-3-yl)methoxy]-7-(propan-2-yloxy)isoquinoline-6-carboxamide;
1-{[(2S,4R)-4-fluoro-5-oxopyrrolidin-2-yl]methoxy}-7-(propan-2-yloxy)isoquinoline-6-carboxamide;
1-{[(2S,4S)-4-fluoro-5-oxopyrrolidin-2-yl]methoxy}-7-(propan-2-yloxy)isoquinoline-6-carboxamide;
1-{[(2S)-4,4-difluoro-5-oxopyrrolidin-2-yl]methoxy}-7-methoxyisoquinoline-6-carboxamide;
1-{[2-(hydroxymethyl)-5-oxopyrrolidin-2-yl]methoxy}-7-(propan-2-yloxy)isoquinoline-6-carboxamide;
1-{[(2S)-4-(hydroxymethyl)-5-oxopyrrolidin-2-yl]methoxy}-7-(propan-2-yloxy)isoquinoline-6-carboxamide;
1-{[(2S,3S)-3-amino-5-oxopyrrolidin-2-yl]methoxy}-7-(propan-2-yloxy)isoquinoline-6-carboxamide;
1-{[(2S,4S)-4-hydroxy-5-oxopyrrolidin-2-yl]methoxy}-7-(propan-2-yloxy)isoquinoline-6-carboxamide;
1-{[(2S,4S)-5-oxo-4-(2,2,2-trifluoroethyl)pyrrolidin-2-yl]methoxy}-7-(propan-2-yloxy)isoquinoline-6-carboxamide;
1-{[(2S,4R)-5-oxo-4-(2,2,2-trifluoroethyl)pyrrolidin-2-yl]methoxy}-7-(propan-2-yloxy)isoquinoline-6-carboxamide;
1-{[(2S)-2-(hydroxymethyl)-5-oxopyrrolidin-2-yl]methoxy}-7-(propan-2-yloxy)isoquinoline-6-carboxamide;
1-{[(2R)-2-(hydroxymethyl)-5-oxopyrrolidin-2-yl]methoxy}-7-(propan-2-yloxy)isoquinoline-6-carboxamide;
1-{[(2S,3S)-5-oxo-3-(trifluoromethyl)pyrrolidin-2-yl]methoxy}-7-(propan-2-yloxy)isoquinoline-6-carboxamide;
1-{(1R)-1 -[(2S)-5-oxopyrrolidin-2-yl]ethoxy}-7-(propan-2-yloxy)isoquinoline-6-carboxamide;
1-{(1S)-1-[(2S)-5-oxopyrrolidin-2-yl]ethoxy}-7-(propan-2-yloxy)isoquinoline-6-carboxamide;
1-[(3R)-morpholin-3-ylmethoxy]-7-(propan-2-yloxy)isoquinoline-6-carboxamide;
7-(propan-2-yloxy)-1-(pyrrolidin-2-ylmethoxy)isoquinoline-6-carboxamide;
1-[(3S)-morpholin-3-ylmethoxy]-7-(propan-2-yloxy)isoquinoline-6-carboxamide;
1-[(1R,6S)-3-azabicyclo[4.1.0]hept-1-ylmethoxy]-7-(propan-2-yloxy)isoquinoline-6-carboxamide;
1-[(1S,6R)-3-azabicyclo[4.1.0]hept-1-ylmethoxy]-7-(propan-2-yloxy)isoquinoline-6-carboxamide;
1-{[(2S,4S)-4-fluoro-4-(hydroxymethyl)-5-oxopyrrolidin-2-yl]methoxy}-7-(propan-2-yloxy)isoquinoline-6-carboxamide;
1-{[(2S,4R)-4-fluoro-4-(hydroxymethyl)-5-oxopyrrolidin-2-yl]methoxy}-7-(propan-2-yloxy)isoquinoline-6-carboxamide;
1-{[(2S,4R)-4-(hydroxymethyl)-5-oxopyrrolidin-2-yl]methoxy}-7-(propan-2-yloxy)isoquinoline-6-carboxamide;
1-{[(2S,4S)-4-(hydroxymethyl)-5-oxopyrrolidin-2-yl]methoxy}-7-(propan-2-yloxy)isoquinoline-6-carboxamide;
1-{[(3aR,4R,6aR)-2,2-dimethyl-6-oxotetrahydro-3aH-[1,3]dioxolo[4,5-c]pyrrol-4-yl]methoxy}-7-(propan-2-yloxy)isoquinoline-6-carboxamide;
4-fluoro-1-{[(2S)-5-oxopyrrolidin-2-yl]methoxy}-7-(propan-2-yloxy)isoquinoline-6-carboxamide;
1-{[(2S)-5-oxopyrrolidin-2-yl]methoxy}-7-(propan-2-yloxy)isoquinoline-6-carboxylic acid;
1-{[(2S,3S,4R)-4-fluoro-3-methyl-5-oxopyrrolidin-2-yl]methoxy}-7-(propan-2-yloxy)isoquinoline-6-carboxamide;
1-{[(2S,4R)-4-fluoro-4-(2-hydroxypropan-2-yl)-5-oxopyrrolidin-2-yl]methoxy}-7-(propan-2-yloxy)isoquinoline-6-carboxamide;
3-methoxy-5-{[(2S)-5-oxopyrrolidin-2-yl]methoxy}naphthalene-2-carboxamide;
1-{[(1S,2S,5R)-4-oxo-3-azabicyclo[3.1.0]hex-2-yl]methoxy}-7-(propan-2-yloxy)isoquinoline-6-carboxamide;
1-{[(1S,5S)-4-oxo-3-azabicyclo[3.1.0]hex-1-yl]methoxy}-7-(propan-2-yloxy)isoquinoline-6-carboxamide;
8-fluoro-1-{[(2S)-5-oxopyrrolidin-2-yl]methoxy}-7-(propan-2-yloxy)isoquinoline-6-carboxamide;
1-{[(2S,4S)-4-fluoro-4-methyl-5-oxopyrrolidin-2-yl]methoxy}-7-methoxyisoquinoline-6-carboxamide;
1-{[(2S)-5-oxopyrrolidin-2-yl]methoxy}-7-(trifluoromethoxy)isoquinoline-6-carboxamide;
1-{[(2S,4R)-4-hydroxy-5-oxopyrrolidin-2-yl]methoxy}-7-(propan-2-yloxy)isoquinoline-6-carboxamide;
1-{[(2S)-1-methyl-5-oxopyrrolidin-2-yl]methoxy}-7-(propan-2-yloxy)isoquinoline-6-carboxamide;
1-{[(2S)-4-(4-hydroxytetrahydro-2H-pyran-4-yl)-5-oxopyrrolidin-2-yl]methoxy}-7-(propan-2-yloxy)isoquinoline-6-carboxamide;
1-{[(2S,4R)-4-hydroxy-5-oxo-4-(2,2,2-trifluoroethyl)pyrrolidin-2-yl]methoxy}-7-(propan-2-yloxy)isoquinoline-6-carboxamide;
1-{[(2S,3S)-4,4-difluoro-3-methyl-5-oxopyrrolidin-2-yl]methoxy}-7-(propan-2-yloxy)isoquinoline-6-carboxamide;
1-{[(2S,4S)-4-hydroxy-5-oxo-4-(2,2,2-trifluoroethyl)pyrrolidin-2-yl]methoxy}-7-(propan-2-yloxy)isoquinoline-6-carboxamide;
1-{[(4S)-1-methyl-2-oxoimidazolidin-4-yl]methoxy}-7-(propan-2-yloxy)isoquinoline-6-carboxamide;
1-{[(5S,6R)-2-oxo-1-azaspiro[4.4]non-6-yl]oxy}-7-(propan-2-yloxy)isoquinoline-6-carboxamide;
1-{[(2S,3S4S)-4-fluoro-3-methyl-5-oxopyrrolidin-2-yl]methoxy}-7-(propan-2-yloxy)isoquinoline-6-carboxamide;
1-{[(2S,3S,4R)-4-fluoro-3-methyl-5-oxopyrrolidin-2-yl]methoxy}-7-methoxyisoquinoline-6-carboxamide;
1-{[(2S,3S,4S)-4-fluoro-3-methyl-5-oxopyrrolidin-2-yl]methoxy}-7-methoxyisoquinoline-6-carboxamide;
4-cyano-1-{[(2S)-5-oxopyrrolidin-2-yl]methoxy}-7-(propan-2-yloxy)isoquinoline-6-carboxamide;
7-(propan-2-yloxy)-1-(pyrrolidin-3-ylmethoxy)isoquinoline-6-carboxamide;
1-{[(2S,4R)-4-(3-hydroxyoxetan-3-yl)-5-oxopyrrolidin-2-yl]methoxy}-7-(propan-2-yloxy)isoquinoline-6-carboxamide;
1-{[(2S,4S)-4-(3-hydroxyoxetan-3-yl)-5-oxopyrrolidin-2-yl]methoxy}-7-(propan-2-yloxy)isoquinoline-6-carboxamide;
5-{[(2S,4S)-4-fluoro-4-methyl-5-oxopyrrolidin-2-yl]methoxy}-3-methoxynaphthalene-2-carboxamide;
4-(aminomethyl)-1-{[(2S)-5-oxopyrrolidin-2-yl]methoxy}-7-(propan-2-yloxy)isoquinoline-6-carboxamide;
1-{[(2R,3R4S)-3-ethyl-4-fluoro-3-hydroxy-5-oxopyrrolidin-2-yl]methoxy}-7-methoxyisoquinoline-6-carboxamide;
1-{[(3S,4S)-3-ethyl-4-fluoro-2-hydroxy-5-oxopyrrolidin-2-yl]methoxy}-7-methoxyisoquinoline-6-carboxamide;
1-{[(2S,3R,4S)-4-fluoro-3-(1-hydroxyethyl)-5-oxopyrrolidin-2-yl]methoxy}-7-methoxyisoquinoline-6-carboxamide;
7-(oxetan-3-yloxy)-1-{[(2S)-5-oxopyrrolidin-2-yl]methoxy}isoquinoline-6-carboxamide;
7-tert-butoxy-1-{[(2S)-5-oxopyrrolidin-2-yl]methoxy}isoquinoline-6-carboxamide;
1-{[(2S,3S)-3-ethyl-4,4-difluoro-5-oxopyrrolidin-2-yl]methoxy}-7-(propan-2-yloxy)isoquinoline-6-carboxamide;
1-{[(4S)-2-oxoimidazolidin-4-yl]methoxy}-7-(propan-2-yloxy)isoquinoline-6-carboxamide;
1-{[(2S,3R,4S)-4-fluoro-3-methyl-5-oxopyrrolidin-2-yl]methoxy}-7-methoxyisoquinoline-6-carboxamide;
7-(cyclopropylmethoxy)-1-{[(2S)-5-oxopyrrolidin-2-yl]methoxy}isoquinoline-6-carboxamide;
1-{[(2S,4R)-4-fluoro-4-(hydroxymethyl)-5-oxopyrrolidin-2-yl]methoxy}-7-methoxyisoquinoline-6-carboxamide;
6-methoxy-4-{[(2S)-5-oxopyrrolidin-2-yl]methoxy}isoquinoline-7-carboxamide
5-{[(2S4R)-4-fluoro-4-(hydroxymethyl)-5-oxopyrrolidin-2-yl]methoxy}-3-methoxynaphthalene-2-carboxamide;
1-{[(2S,3S,4R)-4-fluoro-4-(hydroxymethyl)-3-methyl-5-oxopyrrolidin-2-yl]methoxy}-7-methoxyisoquinoline-6-carboxamide;
6-methoxy-4-{[(2S)-5-oxopyrrolidin-2-yl]methoxy}quinazoline-7-carboxamide;
1-{[(2S,3S4S)-4-fluoro-4-(hydroxymethyl)-3-methyl-5-oxopyrrolidin-2-yl]methoxy}-7-methoxyisoquinoline-6-carboxamide;
7-methoxy-1-{[(2S,3R)-3-methyl-5-oxopyrrolidin-2-yl]methoxy}isoquinoline-6-carboxamide;
1-{[(2S,3S)-3-(hydroxymethyl)-5-oxopyrrolid in-2-yl]methoxy}-7-(propan-2-yloxy)isoquinoline-6-carboxamide;
1-{[(1S,3aS,6aR)-5-methyl-3-oxooctahydropyrrolo[3,4-c]pyrrol-1-yl]methoxy}-7-(propan-2-yloxy)isoquinoline-6-carboxamide;
1-{[(2S,3S)-3-(hydroxymethyl)-5-oxopyrrolidin-2-yl]methoxy}-7-methoxyisoquinoline-6-carboxamide;
1-{[(2S,4S)-4-fluoro-4-(fluoromethyl)-5-oxopyrrolidin-2-yl]methoxy}-7-methoxyisoquinoline-6-carboxamide;
3-methoxy-5-{[(2S,3R)-3-methyl-5-oxopyrrolidin-2-yl]methoxy}naphthalene-2-carboxamide;
5-{[(2S,3S,4S)-4-fluoro-3-methyl-5-oxopyrrolidin-2-yl]methoxy}-3-methoxynaphthalene-2-carboxamide;
1-{[(2S,3R)-3-methyl-5-oxopyrrolidin-2-yl]methoxy}-7-(propan-2-yloxy)isoquinoline-6-carboxamide;
8-fluoro-5-{[(2S,3S,4S)-4-fluoro-3-methyl-5-oxopyrrolidin-2-yl]methoxy}-3-methoxynaphthalene-2-carboxamide;
1-{[(2S)-3,3-dimethyl-5-oxopyrrolidin-2-yl]methoxy}-7-methoxyisoquinoline-6-carboxamide;
1-{[(2R)-3,3-dimethyl-5-oxopyrrolidin-2-yl]methoxy}-7-methoxyisoquinoline-6-carboxamide;
1-{[(2R)-3,3-dimethyl-5-oxopyrrolidin-2-yl]methoxy}-7-(propan-2-yloxy)isoquinoline-6-carboxamide;
1-{[(2S,4R)-4-(cyanomethyl)-5-oxopyrrolidin-2-yl]methoxy}-7-methoxyisoquinoline-6-carboxamide;
1-{[(2S,4S)-4-(cyanomethyl)-5-oxopyrrolidin-2-yl]methoxy}-7-methoxyisoquinoline-6-carboxamide;
1-{[(1S,3aS,6aR)-3-oxooctahydropyrrolo[3,4-c]pyrrol-1-yl]methoxy}-7-(propan-2-yloxy)isoquinoline-6-carboxamide;
7-methoxy-1-{[(4R,5R)-5-methyl-2-oxo-1,3-oxazolidin-4-yl]methoxy}isoquinoline-6-carboxamide;
3-methoxy-5-{[(4R,5R)-5-methyl-2-oxo-1,3-oxazolidin-4-yl]methoxy}naphthalene-2-carboxamide;
7-methoxy-1-{[(4S)-2-oxo-1,3-oxazolidin-4-yl]methoxy}isoquinoline-6-carboxamide;
7-methoxy-1-{[(4R)-2-oxo-1,3-oxazolidin-4-yl]methoxy}isoquinoline-6-carboxamide;
3-methoxy-5-{[(2S,4S)-4-methoxy-5-oxopyrrolidin-2-yl]methoxy}naphthalene-2-carboxamide;
3-methoxy-5-{[(2S,4R)-4-methoxy-5-oxopyrrolidin-2-yl]methoxy}naphthalene-2-carboxamide;
3-methoxy-5-{[(4R)-2-oxo-1,3-oxazolidin-4-yl]methoxy}naphthalene-2-carboxamide
3-methoxy-5-{[(4R5S)-5-methyl-2-oxo-1,3-oxazolidin-4-yl]methoxy}naphthalene-2-carboxamide;
7-methoxy-1-{[(5R)-2-oxo-1,3-oxazolidin-5-yl]methoxy}isoquinoline-6-carboxamide;
7-methoxy-1-{[(4R,5S)-5-methyl-2-oxo-1,3-oxazolidin-4-yl]methoxy}isoquinoline-6-carboxamide;
1-{[(2S,3S4R)-4-fluoro-3,4-dimethyl-5-oxopyrrolidin-2-yl]methoxy}-7-methoxyisoquinoline-6-carboxamide;
1-{[(2S,3S4S)-4-fluoro-3,4-dimethyl-5-oxopyrrolidin-2-yl]methoxy}-7-methoxyisoquinoline-6-carboxamide;
7-methoxy-1-{[(5R)-3-methyl-2-oxo-1,3-oxazolidin-5-yl]methoxy}isoquinoline-6-carboxamide;
5-{[(2S,4R)-4-fluoro-5-oxo-4-(2,2,2-trifluoroethyl)pyrrolidin-2-yl]methoxy}-3-methoxynaphthalene-2-carboxamide;
5-{[(2S,4S)-4-fluoro-5-oxo-4-(2,2,2-trifluoroethyl)pyrrolidin-2-yl]methoxy}-3-methoxynaphthalene-2-carboxamide;
7-methoxy-1-{[(2S4S)-4-methyl-5-oxopyrrolidin-2-yl]methoxy}isoquinoline-6-carboxamide;
7-methoxy-1-{[(2S,4R)-4-methyl-5-oxopyrrolidin-2-yl]methoxy}isoquinoline-6-carboxamide;
1-{[(2S,3S)-3-ethyl-5-oxopyrrolidin-2-yl]methoxy}-7-methoxyisoquinoline-6-carboxamide;
7-methoxy-1-{[(6S)-4-oxo-5-azaspiro[2.4]hept-6-yl]methoxy}isoquinoline-6-carboxamide;
1-{[(2S,3R)-3-ethyl-5-oxopyrrolidin-2-yl]methoxy}-7-methoxyisoquinoline-6-carboxamide;
1-{[(2S,3R,4S)-3,4-dimethyl-5-oxopyrrolidin-2-yl]methoxy}-7-methoxyisoquinoline-6-carboxamide;
1-{[(2S,3R,4R)-3,4-dimethyl-5-oxopyrrolidin-2-yl]methoxy}-7-methoxyisoquinoline-6-carboxamide;
1-{[(2S,3S)-3-(fluoromethyl)-5-oxopyrrolidin-2-yl]methoxy}-7-methoxyisoquinoline-6-carboxamide;
4-{[(2S,3S,4S)-4-fluoro-3-methyl-5-oxopyrrolidin-2-yl]methoxy}-6-methoxyquinoline-7-carboxamide;
1-{[(2S,3S)-3-ethenyl-5-oxopyrrolidin-2-yl]methoxy}-7-methoxyisoquinoline-6-carboxamide;
1-{[(2S,4S)-4-fluoro-5-oxopyrrolidin-2-yl]methoxy}-7-methoxyisoquinoline-6-carboxamide;
1-{[(2S,4R)-4-fluoro-5-oxopyrrolidin-2-yl]methoxy}-7-methoxyisoquinoline-6-carboxamide;
1-{[(2S,3S,4S)-3-(fluoromethyl)-4-methyl-5-oxopyrrolidin-2-yl]methoxy}-7-methoxyisoquinoline-6-carboxamide;
7-methoxy-1-{[(2R)-5-oxopyrrolidin-2-yl]methoxy}isoquinoline-6-carboxamide;
1-{[(2S,4R)-4-ethyl-4-fluoro-5-oxopyrrolidin-2-yl]methoxy}-7-methoxyisoquinoline-6-carboxamide;
1-{[(2S,4S)-4-ethyl-4-fluoro-5-oxopyrrolidin-2-yl]methoxy}-7-methoxyisoquinoline-6-carboxamide;
1-{[(2S)-4-benzyl-5-oxopyrrolidin-2-yl]methoxy}-7-methoxyisoquinoline-6-carboxamide;
4-{[(2S,4S)-4-ethyl-4-fluoro-5-oxopyrrolidin-2-yl]methoxy}-6-methoxyquinoline-7-carboxamide;
4-{[(2S4R)-4-ethyl-4-fluoro-5-oxopyrrolidin-2-yl]methoxy}-6-methoxyquinoline-7-carboxamide;
1-{[(2S,4R)-4-fluoro-4-(fluoromethyl)-5-oxopyrrolidin-2-yl]methoxy}-7-methoxyisoquinoline-6-carboxamide;
6-methooy-4-{[(2S)-5-oxopyrrolidin-2-yl]methoxy}quinoline-7-carboxamide;
1-{[(1R,2S,5S)-6,6-dimethyl-4-oxo-3-azabicyclo[3.1.0]hex-2-yl]methoxy}-7-methoxyisoquinoline-6-carboxamide;
7-methoxy-1-{[(1S,2S,5R)-4-oxo-3-azabicyclo[3.1 .0]hex-2-yl]methoxy}isoquinoline-6-carboxamide;
7-methoxy-1-[(3-methyl-5-oxomorpholin-3-yl)methoxy]isoquinoline-6-carboxamide;
7-methoxy-1-[(4-methyl-2-oxo-1,3-oxazolidin-4-yl)methoxy]isoquinoline-6-carboxamide;
7-methoxy-1-{[(2S,4S)-5-oxo-4-(2,2,2-trifluoroethyl)pyrrolidin-2-yl]methoxy}isoquinoline-6-carboxamide;
4-{[(2S,4S)-4-fluoro-4-methyl-5-oxopyrrolidin-2-yl]methoxy}-6-methoxyquinoline-7-carboxamide;
4-{[(2S,4S)-4-fluoro-4-methyl-5-oxopyrrolidin-2-yl]methoxy}-6-(propan-2-yloxy)quinoline-7-carboxamide;
7-methoxy-1-{[(2S,4R)-5-oxo-4-(2,2,2-trifluoroethyl)pyrrolidin-2-yl]methoxy}isoquinoline-6-carboxamide;
7-methoxy-1-{[(1S,2S,5R)-6-methyl-4-oxo-3-azabicyclo[3.1.0]hex-2-yl]methoxy}isoquinoline-6-carboxamide;
1-{[(2S,3S,4S)-4-fluoro-3-methyl-5-oxopyrrolidin-2-yl]methoxy}-7-methoxyisoquinoline-6-carbonitrile;
1-(cyclopentylmethoxy)-7-methoxyisoquinoline-6-carboxamide;
4-{[(2S,4R)-4-fluoro-4-(2-fluoroethyl)-5-oxopyrrolidin-2-yl]methoxy}-6-methoxyquinoline-7-carboxamide;
1-{[(2R,4R)-4-fluoro-5-oxopyrrolidin-2-yl]methoxy}-7-methoxyisoquinoline-6-carboxamide;
1-{[(2S,4R)-4-fluoro-4-(2-fluoroethyl)-5-oxopyrrolidin-2-yl]methoxy}-7-methoxyisoquinoline-6-carboxamide;
7-methoxy-1-{[(2R,3S)-3-methyl-5-oxopyrrolidin-2-yl]methoxy}isoquinoline-6-carboxamide;
7-ethoxy-1-{[(2S,4S)-4-fluoro-4-methyl-5-oxopyrrolidin-2-yl]methoxy}isoquinoline-6-carboxamide;
6-ethoxy-4-{[(2S,3S,4S)-4-fluoro-3-methyl-5-oxopyrrolidin-2-yl] methoxy}quinoline-7-carboxamide;
6-ethoxy-4-{[(1S,2S,5R)-4-oxo-3-azabicyclo[3.1.0]hex-2-yl]methoxy}quinoline-7-carboxamide;
7-(cyclopropyloxy)-1-{[(2S,3S,4S)-4-fluoro-3-methyl-5-oxopyrrolidin-2-yl]methoxy}isoquinoline-6-carboxamide;
7-ethoxy-1-{[(1S,2S,5R)-4-oxo-3-azabicyclo[3.1.0]hex-2-yl]methoxy}isoquinoline-6-carboxamide;
7-ethoxy-1-{[(2S,3S,4S)-4-fluoro-3-methyl-5-oxopyrrolidin-2-yl]methoxy}isoquinoline-6-carboxamide;
1-{[(2S,4R)-4-fluoro-5-oxo-4-(tetrahydro-2H-pyran-4-yl)pyrrolidin-2-yl]methoxy}-7-methoxyisoquinoline-6-carboxamide;
7-methoxy-1-(((1R,2S,5R,6R)-6-methyl-4-oxo-3-azabicyclo[3.1.0]hexan-2-yl)methoxy)isoquinoline-6-carboxamide;
7-methoxy-1-{[(2S,4S)-5-oxo-4-(tetrahydro-2H-pyran-4-yl)pyrrolidin-2-yl]methoxy}isoquinoline-6-carboxamide;
6-ethoxy-4-{[(2S,4S)-4-fluoro-4-methyl-5-oxopyrrolidin-2-yl]methoxy}quinoline-7-carboxamide;
1-{[(2R,3R,4R)-4-fluoro-3-methyl-5-oxopyrrolidin-2-yl]methoxy}-7-methoxyisoquinoline-6-carboxamide;
1-{[(2S,4S)-4-(4-hydroxytetrahydro-2H-pyran-4-yl)-5-oxopyrrolidin-2-yl]methoxy}-7-methoxyisoquinoline-6-carboxamide;
7-methoxy-1-{[(2S)-4-(oxetan-3-ylidene)-5-oxopyrrolidin-2-yl]methoxy}isoquinoline-6-carboxamide;
6-ethoxy-4-{[(2S)-5-oxopyrrolidin-2-yl]methoxy}quinoline-7-carboxamide;
1-{[(2S,4R)-4-fluoro-4-(methoxymethyl)-5-oxopyrrolidin-2-yl]methoxy}-7-methoxyisoquinoline-6-carboxamide;
6-ethoxy-4-{[(2S,4S)-4-fluoro-4-(fluoromethyl)-5-oxopyrrolidin-2-yl]methoxy}quinoline-7-carboxamide;
7-ethoxy-1-{[(2S,4S)-4-fluoro-4-(fluoromethyl)-5-oxopyrrolidin-2-yl]methoxy}isoquinoline-6-carboxamide;
7-methoxy-1-{[(1S,2S,5R)-1-methyl-4-oxo-3-azabicyclo[3.1.0]hex-2-yl]methoxy}isoquinoline-6-carboxamide;
1-{[(2S,3S,4S)-3-ethyl-4-fluoro-5-oxopyrrolidin-2-yl]methoxy}-7-methoxyisoquinoline-6-carboxamide;
7-methoxy-1-{[(1S,2S,5R)-5-methyl-4-oxo-3-azabicyclo[3.1.0]hex-2-yl]methoxy}isoquinoline-6-carboxamide;
7-methoxy-1-{[(2S,4S)-4-(oxetan-3-yl)-5-oxopyrrolidin-2-yl]methoxy}isoquinoline-6-carboxamide;
1-{[(1S,2S,5R)-1-ethyl-4-oxo-3-azabicyclo[3.1.0]hex-2-yl]methoxy}-7-methoxyisoquinoline-6-carboxamide;
1-{[(1S,2S,5R)-6-ethyl-4-oxo-3-azabicyclo[3.1.0]hex-2-yl]methoxy}-7-methoxyisoquinoline-6-carboxamide;
1-(((1R,2S,5R,6R)-6-ethyl-4-oxo-3-azabicyclo[3.1 .0]hexan-2-yl)methoxy)-7-methoxyisoquinoline-6-carboxamide;
7-methoxy-1-{[(2S)-6-oxopiperidin-2-yl]methoxy}isoquinoline-6-carboxamide;
1-(((1S,2S,5S,6R)-6-(fluoromethyl)-4-oxo-3-azabicyclo[3.1.0]hexan-2-yl)methoxy)-7-methoxyisoquinoline-6-carboxamide;
1-{[(1R,2S,5S)-6-(fluoromethyl)-4-oxo-3-azabicyclo[3.1 .0]hex-2-yl]methoxy}-7-methoxyisoquinoline-6-carboxamide;
1-{[(2S,3S)-3-cyclopropyl-5-oxopyrrolidin-2-yl]methoxy}-7-methoxyisoquinoline-6-carboxamide;
1-(((1R,2S,5R,6R)-6-(2-fluoroethyl)-4-oxo-3-azabicyclo[3.1.0]hexan-2-yl)methoxy)-7-methoxyisoquinoline-6-carboxamide;
7-methoxy-1-{[(1R,2S,5S)-4-oxo-3-azabicyclo[3.1.0]hex-2-yl]methoxy}isoquinoline-6-carboxamide;
7-methoxy-1-{[(1S,2S,5R)-4-oxo-3-azabicyclo[3.2.0]hept-2-yl]methoxy}isoquinoline-6-carboxamide;
1-{[(1R,2S,5S)-5-fluoro-6-methyl-4-oxo-3-azabicyclo[3.1.0]hex-2-yl]methoxy}-7-methoxyisoquinoline-6-carboxamide;
1-(((1S,2S,5S,6R)-5-fluoro-6-methyl-4-oxo-3-azabicyclo[3.1.0]hexan-2-yl)methoxy)-7-methoxyisoquinoline-6-carboxamide;
1-{[(1S,2S,5R)-6,6-dichloro-4-oxo-3-azabicyclo[3.1.0]hex-2-yl]methoxy}-7-methoxyisoquinoline-6-carboxamide;
7-methoxy-1-{[(2S,3R)-5-oxo-3-propylpyrrolidin-2-yl]methoxy}isoquinoline-6-carboxamide;
7-methoxy-1-{[(1S,2S,5S)-6-(methoxymethyl)-4-oxo-3-azabicyclo[3.1.0]hex-2-yl]methoxy}isoquinoline-6-carboxamide;
7-methoxy-1-{[(1S,2S,5S)-6-(methoxymethyl)-4-oxo-3-azabicyclo[3.1.0]hex-2-yl]methoxy}isoquinoline-6-carboxamide;
1-{[(1S,2S,5R)-6-fluoro-4-oxo-3-azabicyclo[3.1.0]hex-2-yl]methoxy}-7-methoxyisoquinoline-6-carboxamide;
4-{[(2S,3S,4S)-3-ethyl-4-fluoro-5-oxopyrrolidin-2-yl]methoxy}-6-methoxyquinoline-7-carboxamide;
1-{[(1R,2S,5S)-5-fluoro-4-oxo-3-azabicyclo[3.1.0]hex-2-yl]methoxy}-7-methoxyisoquinoline-6-carboxamide;
1-{[(2S)-5-oxopyrrolidin-2-yl]methoxy}-7-(prop-2-yn-1-yloxy)isoquinoline-6-carboxamide;
1-{[(2S)-5-oxopyrrolidin-2-yl]methoxy}-7-(propadienyloxy)isoquinoline-6-carboxamide;
1-{[(1R,2S,5S)-6-(difluoromethyl)-4-oxo-3-azabicyclo[3.1.0]hex-2-yl]methoxy}-7-methoxyisoquinoline-6-carboxamide;
3-chloro-6-methoxy-4-{[(1S2S,5R)-6-methyl-4-oxo-3-azabicyclo[3.1.0]hex-2-yl]methoxy}quinoline-7-carboxamide;
1-{[(1R,2S,5S)-5-fluoro-4-oxo-3-azabicyclo[3.2.0]hept-2-yl]methoxy}-7-methoxyisoquinoline-6-carboxamide;
4-{[(1R,2S,5S)-5-fluoro-6-methyl-4-oxo-3-azabicyclo[3.1.0]hex-2-yl]methoxy}-6-methoxyquinoline-7-carboxamide;
4-{[(1S,2S,5R)-6-fluoro-4-oxo-3-azabicyclo[3.1.0]hex-2-yl]methoxy}-6-methoxyquinoline-7-carboxamide;
4-{[(1R,2S,5S)-5-fluoro-4-oxo-3-azabicyclo[3.1,0]hex-2-yl]methoxy}-6-methoxyquinoline-7-carboxamide;
6-methoxy-4-{[(1S,2S,5R)-4-oxo-3-azabicyclo[3.1.0]hex-2-yl]methoxy}quinoline-7-carboxamide;
1-{[(1S,2S,5S)-6-(hydroxymethyl)-4-oxo-3-azabicyclo[3.1.0]hex-2-yl]methoxy}-7-methoxyisoquinoline-6-carboxamide;
1-{[(1S,2S,5S)-6-(hydroxymethyl)-4-oxo-3-azabicyclo[3.1.0]hex-2-yl]methoxy}-7-methoxyisoquinoline-6-carboxamide;
1-{[(2S,3R)-3-ethenyl-5-oxopyrrolidin-2-yl]methoxy}-7-methoxyisoquinoline-6-carboxamide;
7-methoxy-1-{[(4S)-6-oxo-5-azaspiro[2.4]hept-4-yl]methoxy}isoquinoline-6-carboxamide;
4-{[(1R,2S,5S)-6-(fluoromethyl)-4-oxo-3-azabicyclo[3.1.0]hex-2-yl]methoxy}-6-methoxyquinoline-7-carboxamide;
1-(((1R,2S,5R,6R)-6-fluoro-4-oxo-3-azabicyclo[3.1.0]hexan-2-yl)methoxy)-7-methoxyisoquinoline-6-carboxamide;
1-{[(1S,2S,5R)-6-fluoro-6-methyl-4-oxo-3-azabicyclo[3.1.0]hex-2-yl]methoxy}-7-methoxyisoquinoline-6-carboxamide
1-{[(1S,2S,5R)-6-fluoro-6-methyl-4-oxo-3-azabicyclo[3.1.0]hex-2-yl]methoxy}-7-methoxyisoquinoline-6-carboxamide;
1-{[(2S,3S,4R)-3-ethyl-4-fluoro-5-oxopyrrolidin-2-yl]methoxy}-7-methoxyisoquinoline-6-carboxamide;
1-{[(2S,3S,4S)-3-ethyl-4-fluoro-5-oxopyrrolidin-2-yl]methoxy}-7-[(trideutero)methyloxy]isoquinoline-6-carboxamide;
1-{[(2S,3S,4S)-3-ethyl-4-fluoro-5-oxopyrrolidin-2-yl]methoxy}-7-(2-methoxyethoxy)isoquinoline-6-carboxamide;
7-methoxy-1-{[(2S,3R)-3-(methoxymethyl)-5-oxopyrrolidin-2-yl]methoxy}isoquinoline-6-carboxamide;
4-{[(2S,3S,4S)-3-ethyl-4-fluoro-5-oxopyrrolidin-2-yl]methoxy}-6-methoxyquinazoline-7-carboxamide;
1-{[(2S,3S,4S)-3-ethyl-4-methoxy-5-oxopyrrolidin-2-yl]methoxy}-7-methoxyisoquinoline-6-carboxamide;
1-{[(2S,3S,4R)-3-ethyl-4-methoxy-5-oxopyrrolidin-2-yl]methoxy}-7-methoxyisoquinoline-6-carboxamide;
1-{[(2S,3S,4S)-3-(pentadeutero)ethyl-4-fluoro-5-oxopyrrolidin-2-yl]methoxy}-7-methoxyisoquinoline-6-carboxamide;
1-{[(2S,3S)-3-ethyl-4,4-difluoro-5-oxopyrrolidin-2-yl]methoxy}-7-methoxyisoquinoline-6-carboxamide;
1-{[(2S,3R,4R)-3-ethyl-4-fluoro-5-oxopyrrolidin-2-yl]methoxy}-7-methoxyisoquinoline-6-carboxamide;
1-{[(2S,3R)-4,4-difluoro-3-(methoxymethyl)-5-oxopyrrolidin-2-yl]methoxy}-7-methoxyisoquinoline-6-carboxamide;
1-{[(2S,3R,4S)-4-fluoro-3-(methoxymethyl)-5-oxopyrrolidin-2-yl]methoxy}-7-methoxyisoquinoline-6-carboxamide;
7-methoxy-1-{[(2S,3S,4S)-4-methoxy-3-methyl-5-oxopyrrolidin-2-yl]methoxy}isoquinoline-6-carboxamide;
7-methoxy-1-{[(2S,3S,4R)-4-methoxy-3-methyl-5-oxopyrrolidin-2-yl]methoxy}isoquinoline-6-carboxamide;
1-{[(2S,3R,4R)-4-fluoro-3-(methoxymethyl)-5-oxopyrrolidin-2-yl]methoxy}-7-methoxyisoquinoline-6-carboxamide;
1-{[(2S,3S,4S)-3-ethyl-4-hydroxy-5-oxopyrrolidin-2-yl]methoxy}-7-methoxyisoquinoline-6-carboxamide;
1-{[(2S,3S,4R)-3-ethyl-4-hydroxy-5-oxopyrrolidin-2-yl]methoxy}-7-methoxyisoquinoline-6-carboxamide;
7-methoxy-1-{(1S)-1-[(2S)-5-oxopyrrolidin-2-yl]ethoxy}isoquinoline-6-carboxamide;
1-{[(2S,3S)-3-(2-fluoroethyl)-5-oxopyrrolidin-2-yl]methoxy}-7-methoxyisoquinoline-6-carboxamide;
1-{[(2S,3R,4S)-4-amino-3-ethyl-5-oxopyrrolidin-2-yl]methoxy}-7-methoxyisoquinoline-6-carboxamide;
1-{[(2S3R,4R)-4-amino-3-ethyl-5-oxopyrrolidin-2-yl]methoxy}-7-methoxyisoquinoline-6-carboxamide;
1-{[(2S,3S4S)-3-ethyl-4-fluoro-5-oxopyrrolidin-2-yl]methoxy}-7-(propan-2-yloxy)isoquinoline-6-carboxamide;
7-ethoxy-1-{[(2S,3S,4S)-3-ethyl-4-fluoro-5-oxopyrrolidin-2-yl]methoxy}isoquinoline-6-carboxamide;
1-{[(2S,3S,4S)-3-ethyl-4-fluoro-5-oxopyrrolidin-2-yl]methoxy}-4-fluoro-7-methoxyisoquinoline-6-carboxamide;
1-{[(2S,3S,4S)-3-ethyl-4-fluoro-5-oxopyrrolidin-2-yl]methoxy}-8-fluoro-7-methoxyisoquinoline-6-carboxamide;
1-{[(2S,3R)-3-ethyl-5-oxopyrrolidin-2-yl]methoxy}-4-fluoro-7-methoxyisoquinoline-6-carboxamide;
1-{[(2S,3R)-3-ethyl-5-oxopyrrolidin-2-yl]methoxy}-8-fluoro-7-methoxyisoquinoline-6-carboxamide;
4-fluoro-7-methoxy-1-{[(1S,2S,5R)-6-methyl-4-oxo-3-azabicyclo[3.1.0]hex-2-yl]methoxy}isoquinoline-6-carboxamide;
8-fluoro-7-methoxy-1-{[(1S,2S,5R)-6-methyl-4-oxo-3-azabicyclo[3.1.0]hex-2-yl]methoxy}isoquinoline-6-carboxamide;
1-{[(2S,3R)-3-(fluoromethyl)-5-oxopyrrolidin-2-yl]methoxy}-7-methoxyisoquinoline-6-carboxamide;
1-{[(2S,3R,4S)-4-fluoro-3-(fluoromethyl)-5-oxopyrrolidin-2-yl]methoxy}-7-methoxyisoquinoline-6-carboxamide;
1-{[(2S,3S,4S)-3-cyclopropyl-4-fluoro-5-oxopyrrolidin-2-yl]methoxy}-7-methoxyisoquinoline-6-carboxamide;
1-{[(2S,3S,4R)-3-cyclopropyl-4-fluoro-5-oxopyrrolidin-2-yl]methoxy}-7-methoxyisoquinoline-6-carboxamide;
1-{[(2S,3S,4S)-4-fluoro-3-(2-fluoroethyl)-5-oxopyrrolidin-2-yl]methoxy}-7-methoxyisoquinoline-6-carboxamide;
4-(1-methyl-1H-imidazol-4-yl)-1-{[(2S)-5-oxopyrrolidin-2-yl]methoxy}-7-(propan-2-yloxy)isoquinoline-6-carboxamide;
4-(1,2-dimethyl-1H-imidazol-4-yl)-1-{[(2S)-5-oxopyrrolidin-2-yl]methoxy}-7-(propan-2-yloxy)isoquinoline-6-carboxamide;
4-(2-methyl-1H-imidazol-4-yl)-1-{[(2S)-5-oxopyrrolidin-2-yl]methoxy}-7-(propan-2-yloxy)isoquinoline-6-carboxamide;
1-{[(2S,3S,4S)-3-ethyl-4-fluoro-5-oxo(3,4-bisdeutero)pyrrolidin-2-yl]methoxy}-7-methoxyisoquinoline-6-carboxamide;
1-{[(2S,3 R,4R)-4-fluoro-3-(fluoromethyl)-5-oxopyrrolidin-2-yl]methoxy}-7-methoxyisoquinoline-6-carboxamide;
4-(4-methylpyrimidin-2-yl)-1-{[(2S)-5-oxopyrrolidin-2-yl]methoxy}-7-(propan-2-yloxy)isoquinoline-6-carboxamide;
4-(5-chloropyrimidin-2-yl)-1-{[(2S)-5-oxopyrrolidin-2-yl]methoxy}-7-(propan-2-yloxy)isoquinoline-6-carboxamide;
4-(6-oxo-1,6-dihydropyridin-2-yl)-1-{[(2S)-5-oxopyrrolidin-2-yl]methoxy}-7-(propan-2-yloxy)isoquinoline-6-carboxamide; and
4-(2-methylpyrimidin-4-yl)-1-{[(2S)-5-oxopyrrolidin-2-yl]methoxy}-7-(propan-2-yloxy)isoquinoline-6-carboxamide; or a pharmaceutically acceptable salt thereof.

The present invention includes the IRAK4 inhibitors described in WO2017/033093, herein incorporated by reference in its entirety. In another embodiment, the present invention provides a method of treating a patient infected with SARS-CoV-2 comprising administering to the patient in need of such treatment a therapeutically effective amount of a compound selected from the group consisting of: 8-{[(2S,3S,4S)-3-ethyl-4-fluoro-5-oxopyrrolidin-2-yl]methoxy}-2-methoxyquinoline-3-carboxamide; 4-(1,3-oxazol-2-yl)-1-{[(2S)-5-oxopyrrolidin-2-yl]methoxy}-7-(propan-2-yloxy)isoquinoline-6-carboxamide; 4-(4-methyl-1H-imidazol-2-yl)-1-{[(2S)-5-oxopyrrolidin-2-yl]methoxy}-7-(propan-2-yloxy)isoquinoline-6-carboxamide; 4-(1-methyl-1 H-pyrazol-3-yl)-1-{[(2S)-5-oxopyrrolidin-2-yl]methoxy}-7-(propan-2-yloxy)isoquinoline-6-carboxamide; 4-(1-methyl-1H-pyrazol-4-yl)-1-{[(2S)-5-oxopyrrolidin-2-yl]methoxy}-7-(propan-2-yloxy)isoquinoline-6-carboxamide; 4-(4-methyl-1,3-oxazol-2-yl)-1-{[(2S)-5-oxopyrrolidin-2-yl]methoxy}-7-(propan-2-yloxy)isoquinoline-6-carboxamide; 4-(4,5-dimethyl-1,3-oxazol-2-yl)-1-{[(2S)-5-oxopyrrolidin-2-yl]methoxy}-7-(propan-2-yloxy)isoquinoline-6-carboxamide; 4-[4-(hydroxymethyl)-1H-imidazol-2-yl]-1-{[(2S)-5-oxopyrrolidin-2-yl]methoxy}-7-(propan-2-yloxy)isoquinoline-6-carboxamide; 4-(5-methyl-1,3-oxazol-2-yl)-1-{[(2S)-5-oxopyrrolidin-2-yl]methoxy}-7-(propan-2-yloxy)isoquinoline-6-carboxamide; 1-{[(2S,3R)-3-ethyl-5-oxopyrrolidin-2-yl]methoxy}-4-[(phenylsulfonyl)amino]-7-(propan-2-yloxy)isoquinoline-6-carboxamide; 1-{[(2S,3R)-3-ethyl-5-oxopyrrolidin-2-yl]methoxy}-7-(propan-2-yloxy)-4-[(pyridin-3-ylsulfonyl)amino]isoquinoline-6-carboxamide; 1-{[(2S,3R)-3-ethyl-5-oxopyrrolidin-2-yl]methoxy}-4-[(1H-imidazol-4-ylsulfonyl)amino]-7-(propan-2-yloxy)isoquinoline-6-carboxamide; 1-{[(2S,3R)-3-ethyl-5-oxopyrrolidin-2-yl]methoxy}-4-{[(1-methyl-1 H-imidazol-4-yl)sulfonyl]amino}-7-(propan-2-yloxy)isoquinoline-6-carboxamide; 4-{[(1,2-dimethyl-1H-imidazol-4-yl)sulfonyl]amino}-1-{[(2S,3R)-3-ethyl-5-oxopyrrolidin-2-yl]methoxy}-7-(propan-2-yloxy)isoquinoline-6-carboxamide; 4-amino-1-{[(2S,3S,4S)-3-ethyl-4-fluoro-5-oxopyrrolidin-2-yl]methoxy}-7-methoxyisoquinoline-6-carboxamide; 1-{[(4R,7S)-7-fluoro-6-oxo-5-azaspiro[2.4]hept-4-yl]methoxy}-7-(propan-2-yloxy)isoquinoline-6-carboxamide; 1-{[(4S)-7-fluoro-6-oxo-5-azaspiro[2.4]hept-4-yl]methoxy}-7-(propan-2-yloxy)isoquinoline-6-carboxamide; 1-{[(4R,7R)-7-fluoro-6-oxo-5-azaspiro[2.4]hept-4-yl]methoxy}-7-(propan-2-yloxy)isoquinoline-6-carboxamide; 1-(((4S,7R)-7-fluoro-6-oxo-5-azaspiro[2.4]heptan-4-yl)methoxy)-7-isopropoxyisoquinoline-6-carboxamide; 1-{[(4S,7R)-7-fluoro-6-oxo-5-azaspiro[2.4]hept-4-yl]methoxy}-7-methoxyisoquinoline-6-carboxamide; 1-{[(4R,7S)-7-fluoro-6-oxo-5-azaspiro[2.4]hept-4-yl]methoxy}-7-methoxyisoquinoline-6-carboxamide; 1-{[(4R,7R)-7-fluoro-6-oxo-5-azaspiro[2.4]hept-4-yl]methoxy}-7-methoxyisoquinoline-6-carboxamide; 1-{[(4S,7S)-7-fluoro-6-oxo-5-azaspiro[2.4]hept-4-yl]methoxy}-7-methoxyisoquinoline-6-carboxamide; 4-{[(2S,3R)-3-ethyl-5-oxopyrrolidin-2-yl]methoxy}-6-methoxyisoquinoline-7-carboxamide; 4-{[(2S,3S,4S)-3-ethyl-4-fluoro-5-oxopyrrolidin-2-yl]methoxy}-6-methoxyisoquinoline-7-carboxamide; 5-{[(2S,3S,4S)-3-ethyl-4-fluoro-5-oxopyrrolidin-2-yl]methoxy}-3-methoxynaphthalene-2-carboxamide; (3S,6R)-5-oxo- 2,3,4,5,6,7,9,10-octahydro-12,14-(ethanediylidene)-3,6-methanopyrido[2,3-l][1,4,11,8]trioxazacyclopentadecine-19-carboxamide; 7-methoxy-1-[(3-oxo-2-azabicyclo[3.1.0]hex-1-yl)methoxy]isoquinoline-6-carboxamide; 7-methoxy-1-{[(1S,5S)-3-oxo-2-azabicyclo[3.1.0]hex-1-yl]methoxy}isoquinoline-6-carboxamide; 7-methoxy-1-{[(1R,5R)-3-oxo-2-azabicyclo[3.1.0]hex-1-yl]methoxy}isoquinoline-6-carboxamide; 5-{[(2S,3S,4S)-3-ethyl-4-fluoro-5-oxopyrrolidin-2-yl]methoxy}-3-methoxy-1,6-naphthyridine-2-carboxamide; and 1-{[(2S,3S,4S)-4-fluoro-3-methyl-5-oxopyrrolidin-2-yl]methoxy}-7-methoxy-N-methylisoquinoline-6-carboxamide; or pharmaceutically acceptable salt thereof.

The present invention includes the IRAK4 inhibitors described in U.S. Pat. 7592443; 7745612; 8217162; 9255110; 9586948; 9598440; 9617282; 9732095; 9926330; 9932350; 9943516; 9969749; 10023589; 10040798; 10047104; 10059708; 10155765; 10160753; 10174000; 10246456; 10316018; 10329294; 10329295; 10562902; and 10577367, all of which are herein incorporated by reference in their entirety. Accordingly, in another embodiment, the present invention provides a method of treating a patient infected with SARS-CoV-2 comprising administering to the patient in need of such treatment a therapeutically effective amount of a compound as described in U.S. Pats. 7592443; 7745612; 8217162; 9255110; 9586948; 9598440; 9617282; 9732095; 9926330; 9932350; 9943516; 9969749; 10023589; 10040798; 10047104; 10059708; 10155765; 10160753; 10174000; 10246456; 10316018; 10329294; 10329295; 10562902; and 10577367; including the pharmaceutically acceptable salts of the specifically named compounds and the pharmaceutically acceptable solvates/hydrates of said specifically named compounds and salts.

The invention also provides the method, wherein said therapeutically effective amount is about 0.01 to about 100 mg/kg of body weight/day, or more preferably about 0.1 to about 10.0 mg/kg, in a single dose or as divided doses administered two, three or four times per day. The invention also provides the method, wherein said therapeutically effective amount is about 400 mgs administered QD. The invention further provides the method, wherein said therapeutically effective amount is about 200 mgs administered Q6H.

In therapeutic use for treating disorders in a patient or subject, a compound of the present invention or its pharmaceutical compositions can be administered orally, parenterally, topically, rectally, transmucosally, or intestinally. Parenteral administrations include indirect injections to generate a systemic effect or direct injections to the afflicted area. Topical administrations include the treatment of skin or organs readily accessible by local application, for example, eyes or ears. It also includes transdermal delivery to generate a systemic effect. The rectal administration includes the form of suppositories. The preferred routes of administration are oral and parenteral.

Pharmaceutical compositions of the present invention may be manufactured by methods well known in the art, e.g., by means of conventional mixing, dissolving, granulation, levigating, emulsifying, encapsulating, entrapping, lyophilizing processes or spray drying.

Pharmaceutical compositions for use in accordance with the present invention may be formulated in conventional manner using one or more pharmaceutically acceptable carriers comprising excipients, diluents, and auxiliaries, which facilitate processing of the active compound into preparations, which can be used pharmaceutically. Proper formulation is dependent upon the route of administration chosen. Pharmaceutically acceptable excipients and carriers are generally known to those skilled in the art and are thus included in the instant invention. Such excipients and carriers are described, for example, in Remington’s Pharmaceutical Sciences, Mack Pub. Co., New Jersey (1991). The formulations of the invention can be designed to be short-acting, fast-releasing, long-acting, and sustained-releasing. Thus, the pharmaceutical formulations can also be formulated for controlled release or for slow release.

Pharmaceutical compositions suitable for use in the present invention include compositions wherein the active ingredients are contained in an amount sufficient to achieve the intended purpose, i.e., treatment of a patient infected with SARS-CoV-2. More specifically, a therapeutically effective amount means an amount of compound effective to prevent, alleviate or ameliorate symptoms/signs of the disease or prolong the survival of the subject being treated.

The quantity of active component, which is the compound of this invention, in the pharmaceutical composition and unit dosage form thereof, may be varied or adjusted widely depending upon the manner of administration, the potency of the particular compound and the desired concentration. Determination of a therapeutically effective amount is well within the capability of those skilled in the art. Generally, the quantity of active component will range between 0.01% to 99% by weight of the composition.

Generally, a therapeutically effective amount of dosage of active component will be in the range of about 0.01 to about 100 mg/kg of body weight/day, preferably about 0.1 to about 10 mg/kg of body weight/day, more preferably about 0.3 to 3 mg/kg of body weight/day, even more preferably about 0.3 to 1.5 mg/kg of body weight/day It is to be understood that the dosages may vary depending upon the requirements of each subject and the severity of the disorders or diseases being treated.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

In another embodiment, the present invention provides a method of treating a patient infected with SARS-CoV-2 comprising administering to the patient in need of such treatment a therapeutically effective amount of a pharmaceutical combination comprising tofacitinib or a pharmaceutically acceptable salt thereof, and PF-06650833 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising tofacitinib or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising PF-06650833 or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising tofacitinib or a pharmaceutically acceptable salt thereof, PF-06650833 or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising azithromycin and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising azithromycin and at least one compound selected from tofacitinib or a pharmaceutically acceptable salt thereof, PF-06650833, or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising hydroxychloroquine; and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising hydroxychloroquine; and at least one compound selected from tofacitinib or a pharmaceutically acceptable salt thereof, PF-06650833, or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising chloroquine; and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising chloroquine; and at least one compound selected from tofacitinib or a pharmaceutically acceptable salt thereof, PF-06650833, or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound selected from ruxolitinib, baricitinib, oclacitinib, fedratinib, upadacitinib and peficitinib; and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound selected from ruxolitinib, baricitinib, oclacitinib, fedratinib, upadacitinib and peficitinib; and at least one compound selected from tofacitinib or a pharmaceutically acceptable salt thereof, PF-06650833, or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound selected from filgotinib, decernotinib, cerdulatinib, gandotinib, lestaurtinib, momelotinib, and pacritinib; and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound selected from filgotinib, decernotinib, cerdulatinib, gandotinib, lestaurtinib, momelotinib, and pacritinib; and at least one compound selected from tofacitinib or a pharmaceutically acceptable salt thereof, PF-06650833, or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound selected from adalimumab, infliximab, certolizumab, golimumab, and vedolizumab; and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound selected from adalimumab, infliximab, certolizumab, golimumab, and vedolizumab; and at least one compound selected from tofacitinib or a pharmaceutically acceptable salt thereof, PF-06650833, or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound selected from abrilada, hadlima, hyrimoz, cyltezo, and Amjevita; and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound selected from abrilada, hadlima, hyrimoz, cyltezo, and Amjevita; and at least one compound selected from tofacitinib or a pharmaceutically acceptable salt thereof, PF-06650833, or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising etanercept; and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising etanercept; and at least one compound selected from tofacitinib or a pharmaceutically acceptable salt thereof, PF-06650833, or a pharmaceutically acceptable salt thereof, and PF-06651600 or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound described in U.S. Pat. 9884876, herein incorporated by reference in its entirety; and at least one compound selected from a JAK inhibitor or a pharmaceutically acceptable salt thereof, an IRAK4 inhibitor or a pharmaceutically acceptable salt thereof, and a TYK2 inhibitor or a pharmaceutically acceptable salt thereof.

In another embodiment, the present invention provides a method of treating a patient infected with SARS-CoV-2 comprising administering to the patient in need of such treatment a therapeutically effective amount of a pharmaceutical combination comprising at least one compound described in U.S. Pat. 9884876, herein incorporated by reference in its entirety; and at least one compound selected from tofacitinib or a pharmaceutically acceptable salt thereof, PF-06650833, or a pharmaceutically acceptable salt thereof, and PF-06651600 or a pharmaceutically acceptable salt thereof.

Suitable agents for use in combination therapy with a JAK inhibitor, IRAK4 inhibitor, or TYK2 inhibitor as set forth herein, or a pharmaceutically acceptable salt thereof, or pharmaceutically acceptable solvate or hydrate of said compound or salt, in the treatment of COVID-19 disease, include: a 5-lipoxygenase activating protein (FLAP) antagonist; a leukotriene antagonist (LTRA) such as an antagonist of LTB₄, LTC₄, LTD₄, LTE₄, CysLT₁ or CysLT₂, e.g., montelukast or zafirlukast; a histamine receptor antagonist, such as a histamine type 1 receptor antagonist or a histamine type 2 receptor antagonist, e.g., loratidine, fexofenadine, desloratidine, levocetirizine, methapyrilene or cetirizine; an α1-adrenoceptor agonist or an α2-adrenoceptor agonist, e.g., phenylephrine, methoxamine, oxymetazoline or methylnorephrine; a muscarinic M3 receptor antagonist, e.g. tiotropium or ipratropium; a dual muscarinic M3 receptor antagononist/β2 agonist; a PDE inhibitor, such as a PDE3 inhibitor, a PDE4 inhibitor or a PDE5 inhibitor, e.g., theophylline, sildenafil, vardenafil, tadalafil, ibudilast, cilomilast or roflumilast; sodium cromoglycate or sodium nedocromil; a cyclooxygenase (COX) inhibitor, such as a non-selective inhibitor (e.g., aspirin or ibuprofen) or a selective inhibitor (e.g. celecoxib or valdecoxib); a glucocorticosteroid, e.g., fluticasone, mometasone, dexamethasone, prednisolone, budesonide, ciclesonide or beclamethasone; an anti-inflammatory monoclonal antibody, e.g., infliximab, adalimumab, tanezumab, ranibizumab, bevacizumab or mepolizumab; a β2 agonist, e.g., salmeterol, albuterol, salbutamol, fenoterol or formoterol, particularly a long-acting β2 agonist; an intigrin antagonist, e.g., natalizumab; an adhesion molecule inhibitor, such as a VLA-4 antagonist; a kinin B₁ or B₂ receptor antagonist; an immunosuppressive agent, such as an inhibitor of the IgE pathway (e.g., omalizumab) or cyclosporine; a matrix metalloprotease (MMP) inhibitor, such as an inhibitor of MMP-9 or MMP-12; a tachykinin NK₁, NK₂ or NK₃ receptor antagonist; a protease inhibitor, such as an inhibitor of elastase, chymase or catheopsin G; an adenosine A₂ₐ receptor agonist; an adenosine A_{2b} receptor antagonist; a urokinase inhibitor; a dopamine receptor agonist (e.g., ropinirole), particularly a dopamine D2 receptor agonist (e.g., bromocriptine); a modulator of the NF_{K}B pathway, such as an IKK inhibitor; a further modulator of a cytokine signalling pathway such as an inhibitor of syk kinase, p38 kinase, SPHK-1 kinase, Rho kinase, EGF-R or MK-2; a mucolytic, mucokinetic or anti-tussive agent; an antibiotic; an antiviral agent; a vaccine; a chemokine; an epithelial sodium channel (ENaC) blocker or Epithelial sodium channel (ENaC) inhibitor; a nucleotide receptor agonist, such as a P2Y2 agonist; a thromboxane inhibitor; niacin; a 5-lipoxygenase (5-LO) inhibitor, e.g., Zileuton; an adhesion factor, such as VLAM, ICAM or ELAM; a CRTH2 receptor (DP₂) antagonist; a prostaglandin D₂ receptor (DP₁) antagonist; a haematopoietic prostaglandin D2 synthase (HPGDS) inhibitor; interferon-β; a soluble human TNF receptor, e.g., Etanercept; a HDAC inhibitor; a phosphoinositide 3-kinase gamma (Pl3K_{Y}) inhibitor; a phosphoinositide 3-kinase delta (Pl3Kδ) inhibitor; a CXCR-1 or a CXCR-2 receptor antagonist; an IRAK-4 inhibitor; and, a TLR-4 or TLR-9 inhibitor, including the pharmaceutically acceptable salts of the specifically named compounds and the pharmaceutically acceptable solvates of said specifically named compounds and salts.

The compounds of the invention may be prepared by any method known in the art. In particular, the compounds of the invention can be prepared by the procedures described by reference to the prior art references in which they are disclosed.

For those compounds which inhibit JAK1 specifically, including N-{cis-3-[methyl(7H-pyrrolo[2,3-d]pyrimidin-4-yl)amino]cyclobutyl}-propane-1-sulfonamide, preparative methods are disclosed in U.S. Pat. No. 9,035,074, the contents of which are incorporated herein in their entirety.

For those compounds which inhibit Tyk2/JAK1 specifically, including [(1S)-2,2-difluorocyclo-propyl][(1R,5S)-3-{2-[(1-methyl-1H-pyrazol-4-yl)amino]pyrimidin-4-yl}-3,8-diazabicyclo[3.2.1]oct-8-yl]methanone, preparative methods are disclosed in U.S. Pat. No. 9,663,526, the contents of which are incorporated herein in their entirety.

For those compounds which inhibit Tyk2 specifically, including (1r,3r)-3-(cyanomethyl)-3-(4-(6-(1-methyl-1 H-pyrazol-4-yl)pyrazolo[1 ,5-a]pyrazin-4-yl)-1 H-pyrazol-1-yl)cyclobutane-1-carbonitrile, preparative methods are disclosed in U.S. Pat. No. 10,144,738, the contents of which are incorporated herein in their entirety.

### Definitions

The term “PF-06650833,” as used herein, means 1-(((2S,3S,4S)-3-Ethyl-4-fluoro-5-oxopyrrolidin-2-yl)methoxy)-7-methoxyisoquinoline-6- carboxamide having the structure

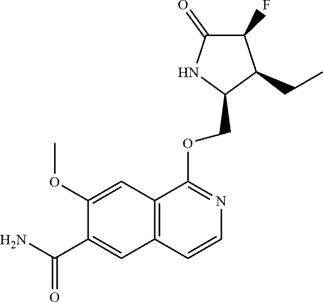 [SMILES: CC[C@@H]1[C@H](F)C(=O)N[C@@H]1COc1nccc2cc(C(N)=O)c(OC)cc12]

and includes any pharmaceutically acceptable crystalline or amorphous form including hydrates, solvates, co-crystals, salts and combinations thereof. Certain forms of 1-(((2S,3S,4S)-3-ethyl-4-fluoro-5-oxopyrrolidin-2-yl)methoxy)-7-methoxyisoquinoline-6- carboxamide may be prepared following the experimental procedures disclosed in WO2015/150995, Org. Process Res. Dev. 2018, 22, 1835-1845, and IP.COM disclosure no.: IPCOM000256080D (2018-NOV-02), all three documents are herein incorporated by reference in their entirety.

The term “PF-06651600,” as used herein, means 1-((2S,5R)-5-((7H-Pyrrolo[2,3-d]pyrimidin-4-yl)amino)-2-methylpiperidin-1-yl)prop-2-en-1-one having structure

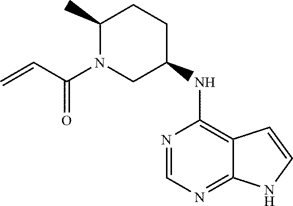 [SMILES: C=CC(=O)N1C[C@H](Nc2ncnc3[nH]ccc23)CC[C@@H]1C]

and includes any pharmaceutically acceptable crystalline or amorphous form including hydrates, solvates, co-crystals, salts and combinations thereof. Certain forms of 1-((2S,5R)-5-((7H-pyrrolo[2,3-d]pyrimidin-4-yl)amino)-2-methylpiperidin-1-yl)prop-2-en-1-one may be prepared following the experimental procedures disclosed in WO2015/083028 and Thorarensen et al., J. Med. Chem. 2017, 60, 1971-1993, both documents are herein incorporated by reference in their entirety.

The term “tofacitinib,” as used herein, means 3-((3R,4R)-4-Methyl-3-(methyl(7H-pyrrolo[2,3-d]pyrimidin-4-yl)amino)piperidin-1-yl)-3-oxopropanenitrile having structure

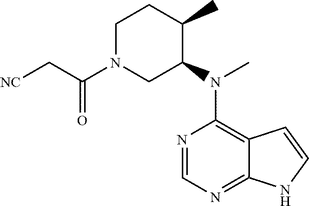

and includes any pharmaceutically acceptable crystalline or amorphous form including hydrates, solvates, co-crystals, salts and combinations thereof. A preferred salt is the citrate salt approved in the U.S. under the brand XELJANZ™ and XELJANZ XR™. Certain forms of tofacitinib may be prepared following the experimental procedures disclosed in WO01/042246, WO02/096909, and WO03/048162, all three documents are herein incorporated by reference in their entirety. The immediate release 10 mg dose of tofacitinib twice daily (BID) may be administered to the patient in need of such treatment as two immediate release 5 mg tablets twice daily (5 mg tablets x 2, BID) wherein the 5 mg tablets are taken together or sequentially.

The compound “(R)-3-(4-(4-(1-(pentan-3-yl)-1H-pyrazol-4-yl)pyrazolo[1 ,5-a]pyrazin-6-yl)-1H-pyrazol-1-yl)propane-1 ,2-diol,” as used herein, has the following structure

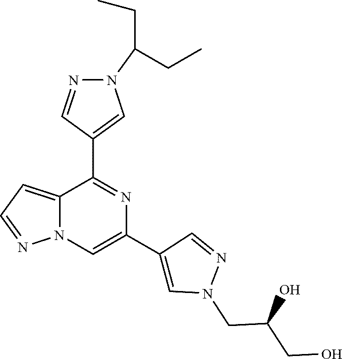 [SMILES: CCC(CC)n1cc(-c2nc(-c3cnn(C[C@@H](O)CO)c3)cn3nccc23)cn1]

and includes any pharmaceutically acceptable crystalline or amorphous form including hydrates, solvates, co-crystals, salts and combinations thereof.Certain forms of (R)-3-(4-(4-(1-(pentan-3-yl)-1H-pyrazol-4-yl)pyrazolo[1 ,5-a]pyrazin-6-yl)-1 H-pyrazol-1-yl)propane-1,2-diol may be prepared following the experimental procedures disclosed in WO16/090285, herein incorporated by reference in it’s entirety.

The term “BID,” as used herein means administration of drug twice a day to patients.

The term “QD,” as used herein means administration of drug once a day to patients.

The term “Q6H,” as used herein means administration of drug every 6 hours or four times a day to patients.

The term “immune” or “immune system,” as used herein means the innate and adaptive immune systems.

The term “patient” or “subject,” as used herein, means a human being in need of the treatments or therapies as described herein.

The term “treating” or “treatment” means an alleviation of symptoms associated with COVID-19 disease, or halt of further progression or worsening of those symptoms, including syndrome coronavirus 2 and acute respiratory distress syndrome (ARDS). Depending on the condition of the patient, the term “treatment” as used herein may include one or more of curative, palliative and prophylactic treatment. Treatment can also include administering a pharmaceutical formulation of the present invention in combination with other therapies.

The term “therapeutically-effective” indicates the capability of an agent to prevent, or improve the severity of COVID-19 disease while avoiding adverse side effects typically associated with alternative therapies. The phrase “therapeutically-effective” is to be understood to be equivalent to the phrase “effective for the treatment, prevention, or amelioration”, and both are intended to qualify the amount of each agent for use in the combination therapy which will achieve the goal of improvement in the severity of COVID-19 disease, or pain or other symptom thereof, and the frequency of incidence over treatment of each agent by itself, while avoiding adverse side effects typically associated with alternative therapies.

“Pharmaceutically acceptable” means suitable for use in a “patient” or “subject.”

### Example 1

### 10 Mg Tofacitinib BID Phase 2 Clinical Study

The objective of the study will be to assess the safety and efficacy of tofacitinib in treating patients with COVID-19 pneumonia and ARDS at time of presentation. Inhibition of JAK/STAT signaling by tofacitinib will reduce excessive pro-inflammatory signaling in ARDS due to SARS-CoV-2 and mitigate further lung injury.

The study will be a randomized, open-label, contemporary case-controlled, parallel group Phase 2 study of the efficacy and safety of tofacitinib in hospitalized adult patients with COVID-19 and ARDS, and requiring mechanical ventilation. Patients randomized to active treatment will receive 10 mg tofacitinib twice daily, administered orally, for up to 14 days, or until discharge from the hospital or intensive care unit (ICU). All dosing of study drug will be in addition to current standard of care treatment, that must include some treatment targeting SARS-CoV-2, (e.g., anti-malarial drugs such as chloroquine / hydroxychloroquine, and anti-viral therapies, some of which may also be experimental). A comparator group will consist of patients who will only receive standard of care treatment. Patients will be assessed daily while hospitalized for a variety of clinical, safety, PK (as available), and laboratory parameters

The inclusion criteria will be: male or female adult patients with laboratory-confirmed novel coronavirus (SARS-CoV-2) infection < 72 hours prior to randomization; a clinical and, as available, radiographic picture consistent with ARDS; and a requirement for mechanical ventilation, ECMO, or high flow ventilatory devices.

The exclusion criteria will be: other medical condition other than COVID-19 or laboratory abnormality that may increase the risk of study participation or, in the investigator’s judgment, make the participant inappropriate for the study, e.g. acute coronary syndrome, chronic infections such as TB and HIV; proven bacterial pneumonia, other serious infection, sepsis, and/or septic shock; alanine transaminase/aspartate transaminase (ALT/AST) > 5 times the upper limit of normal; estimated glomerular filtration rate (eGFR) < 30 mL/min/1.73 m²);pregnancy; and anticipated survival < 72 hours as assessed by the Investigator.

The primary endpoint will be the percentage of patients in each of a 6-point ordinal scale of disease severity at end of treatment (Day 15). A sample size of x patients (1:1 randomization) will have 80% power to detect an odds ratio of xx with two-sided alpha=0.1 and power=80%. Analysis of the primary endpoint will use the proportional odds model.

Secondary endpoints will include: the percentage of patients in each of an ordinal scale of disease severity with time (Days 1-15); the proportion of patients requiring ICU admission and mechanical ventilatory support; the change in National Early Warning Score (NEWS) from baseline; and the safety as assessed by reporting of adverse events, changes in clinical laboratory parameters (e.g., Hb, ALT, AST, Scr, bilirubin).

The safety and clinical pharmacology of tofacitinib has been well characterized. The pharmacodynamics (PD) of the tofacitinib, in terms of cytokine inhibition and biomarkers of pharmacologic activity, have been characterized in in vitro studies, in animal models, and in vivo clinical studies. These data indicate rapid onset, followed by sustained pharmacologic activity over the dosing period. At a 10 mg BID dose, approximately 80% suppression of IL-6 may be expected, in addition to substantial inhibition of multiple other pro-inflammatory cytokines, such as IFNγ, IL-15, IL-21, and IL-27, supporting the use of tofacitinib 10 mg BID for prevention of overexpression of cytokines in COVID-19 infected patients. Given the high mortality in COVID-19 complicated by ARDS, and the overall case mortality rate in in COVID-19 patients, a tofacitinib dose of 10 mg immediate release formulation BID is expected to provide maximal cytokine suppression while maintaining an overall positive benefit-risk profile.

### Example 2

### 400 Mg PF-06650833 QD Phase 2 Clinical Study

The objective of the study will be to assess the safety and efficacy of PF-06650833 in treating patients with COVID-19 pneumonia requiring hospital admission for oxygenation or ventilatory support but not requiring mechanical ventilation at time of presentation. Inhibition of IL-1 receptor and TLR family signaling by PF-06650833 will reduce excessive pro-inflammatory responses to SARS-CoV-2 and mitigate lung injury leading to ARDS.

The study will be a randomized, open-label, contemporary case-controlled, parallel group Phase 2 study of the efficacy and safety of PF-06650833 in hospitalized adult patients with COVID-19 and pneumonia, who do not need mechanical ventilation to maintain adequate oxygenation. Patients randomized to active treatment will receive PF-06650833 400 mg modified release (MR) tablets once daily (QD) administered orally, under fasted conditions, for up to 14 days, or until discharge from the hospital or intensive care unit (ICU). All dosing of study drug will be in addition to current standard of care treatment, that must include some treatment targeting SARS-CoV-2 (e.g., anti-malarial drugs such as chloroquine/hydroxychloroquine, and / or anti-viral therapies, some of which may also be experimental). A comparator group will consist of patients who will only receive standard of care treatment. Patients will be assessed daily while hospitalized for a variety of clinical, safety, PK (as available), and laboratory parameters.

The inclusion criteria will be: male or female adult patients with laboratory-confirmed novel coronavirus (SARS-CoV-2) infection < 72 hours prior to randomization; and evidence of pneumonia assessed by either radiographic infiltrates by imaging (chest x-ray, CT scan, etc.) or clinical assessment (evidence of rales/crackles on exam) and SpO₂ ≤ 94% on room air.

The exclusion criteria will be: other medical condition other than COVID-19 or laboratory abnormality that may increase the risk of study participation or, in the investigator’s judgment, make the participant inappropriate for the study, e.g. acute coronary syndrome, chronic infections such as TB and HIV; proven bacterial pneumonia, other serious infection, sepsis, and/or septic shock; alanine transaminase/aspartate transaminase (ALT/AST) >5 times the upper limit of normal; estimated glomerular filtration rate (eGFR) <60 mL/min/1.73 m²); pregnancy; and anticipated survival <72 hours as assessed by the Investigator.

The primary endpoint will be the percentage of patients in each of a 6-point ordinal scale of disease severity at end of treatment (Day 15). A sample size of x patients (1:1 randomization) will have 80% power to detect an odds ratio of xx with two-sided alpha=0.1 and power=80%. Analysis of the primary endpoint will use the proportional odds model.

Secondary endpoints will include: the percentage of patients in each of an ordinal scale of disease severity with time (Days 1-15); the proportion of patients requiring ICU admission and mechanical ventilatory support; the change in National Early Warning Score (NEWS) from baseline; and the safety as assessed by reporting of adverse events, changes in clinical laboratory parameters (e.g., Hb, ALT, AST, Scr, bilirubin).

PF-06650833 has been well-tolerated with an acceptable safety profile based on clinical studies conducted to date in healthy participants and patients with RA. Nonclinical efficacy and clinical biomarker data suggest that a continuous high degree of inhibition of IRAK4 is required for efficacy. An exploratory exposure response model suggests that maintaining PF-06650833 minimum observed concentration (Cₘᵢₙ) above the in vitro 90% inhibitory concentration ([IC₉₀]) is desirable for maximal CRP reduction. A dose of 400 mg PF-06650833 MR tablets administered once QD expected to inhibit TLR7/8 ligand-induced IL-6 production by approximately 90 and 95% at Cₘᵢₙ and Cₘₐₓ, respectively, which would translate to about 50-60% reduction in CRP. Given the high mortality in COVID-19 complicated by ARDS, and the overall case mortality rate in in COVID-19 patients, a PF-06650833 dose of 400 mg MR tablets QD, fasted, is expected to provide maximal cytokine suppression while maintaining an overall positive benefit-risk profile.

### Example 3

### 200 Mg PF-06650833 Q6H Phase 2 Clinical Study

The objective of the study will be to assess the safety and efficacy of PF-06650833 in treating patients with COVID-19 pneumonia and ARDS at time of presentation. Inhibition of IL- 1 receptor and TLR family signaling by PF-06650833 will reduce excessive inflammatory signaling in ARDS due to SARS-CoV-2 and mitigate further lung injury and death.

The study will be a randomized, open-label, single arm, historical-controlled, Phase 2 study of the efficacy and safety of PF-06650833 in hospitalized adult patients with COVID-19 and ARDS, and who need mechanical ventilation to maintain adequate oxygenation. Patients randomized to active treatment will receive PF-06650833 200 mg immediate release (IR) formulation every 6 hours (Q6H), administered orally, for up to 28 days, or until discharge from the hospital or intensive care unit (ICU). All dosing of study drug will be in addition to current standard of care treatment, that must include some treatment targeting SARS-CoV- (e.g., anti-malarial drugs such as chloroquine / hydroxychloroquine, and / or anti-viral therapies, some of which may also be experimental). The comparator group will consist of historical control ARDS patients who only received standard of care treatment. Patients will be assessed daily while hospitalized for a variety of clinical, safety, PK (as available), and laboratory parameters.

The inclusion criteria will be: male or female adult patients with laboratory-confirmed novel coronavirus (SARS-CoV-2) infection <72 hours prior to randomization; clinical findings and an imaging study consistent with ARDS; and a requirement for mechanical ventilation, ECMO, or high flow ventilatory devices.

The exclusion criteria will be: other medical condition other than COVID-19 or laboratory abnormality that may increase the risk of study participation or, in the investigator’s judgment, make the participant inappropriate for the study, e.g. acute coronary syndrome, chronic infections such as TB and HIV; proven bacterial pneumonia, other serious infection, sepsis, and/or septic shock; alanine transaminase/aspartate transaminase (ALT/AST) >5 times the upper limit of normal; estimated glomerular filtration rate (eGFR) <60 mL/min/1.73 m²); pregnancy; and anticipated survival <72 hours as assessed by the Investigator.

The primary endpoint will be the overall survival rate (Day 28). Assuming a true survival rate of 75% for IRAK4+SOC, an historical control rate of 50%, with a one-sided alpha of 0.1 and 80% power and exact binomial test, a sample size of 21 patients are needed. Study will be statistically significant if at least 14 patients survive out of 21 patients.

Secondary endpoints will include: the percentage of patients in each of a 6-point ordinal scale of disease severity with time; the proportion of patients requiring ICU admission and mechanical ventilatory support; the change in National Early Warning Score (NEWS) from baseline; and the safety as assessed by reporting of adverse events, changes in clinical laboratory parameters (e.g., hemoglobin, liver transaminases, serum creatinine, bilirubin).

PF-06650833 has been well-tolerated with an acceptable safety profile based on clinical studies conducted to date in healthy participants and patients with RA. Our exploratory exposure-response model suggests that maintaining PF-06650833 minimum observed concentration (Cₘᵢₙ) above the in vitro 90% inhibitory concentration ([IC₉₀]) is desirable for maximal CRP reduction. A dose of 200 mg PF-06650833 IR suspension administered Q6H expected to inhibit TLR7/8 ligand-induced IL-6 production by more than 90%, which would translate to about 50-60% reduction in CRP. Given the high mortality in COVID-19 complicated by ARDS, and the overall case mortality rate in COVID-19 patients, a PF-06650833 dose of 200 mg IR formulation Q6H is expected to provide maximal cytokine suppression while maintaining an overall positive benefit-risk profile.

### Example 4

### 400 Mg PF-06650833 QD Phase 2 Clinical Study (28 Days), An Exploratory, Randomized, Open Label, Case-Controlled, Single Center, Phase 2 Trial Assessing the Efficacy and Safety of PF-06650833 in Hospitalized Patients with Covid-19 Pneumonia and Exuberant Inflammation

Coronavirus disease - 2019 (COVID-19) is a viral disease caused by a novel coronavirus, SARS-CoV-2, that can cause a severe acute respiratory syndrome (ARDS). Many patients rapidly (within 1 - 2 weeks of infection) develop dyspnea and pneumonia and require hospitalization for respiratory support. Of these hospitalized patients, 20-30% have required admission to intensive care units (ICUs) for ventilatory support due to development of ARDS, with ventilatory failure being a major cause of overall mortality due to COVID-19. There are currently no established treatments for COVID-19, and there is therefore a dire need for therapeutic interventions that prevent the progression of infection to severe disease and death. Preclinical data from beta-coronaviruses similar to SARS-CoV-2 suggest that the pathogenic characteristics of progressive disease are dominated by an intense inflammatory response. The ultimate result is progression destruction of the alveolar epithelium leading to ARDS. Moreover, the exudative phase of ARDS is thought to be due to an influx of myeloid cells (neutrophils and macrophages) and elevations of inflammatory cytokines, with higher levels of both IL-6 and IL-8 levels being correlated with increased mortality. Therefore, immunomodulatory therapy may be beneficial in reducing the deleterious effects of lung inflammation and mitigating progressive lung injury.

An important consideration in selecting an immunomodulatory target in this setting is the desire to be confident that inhibiting the pathway will permit ongoing viral clearance, while blocking harmful collateral damage. Interleukin-1 receptor associated kinase (IRAK) 4 is a serine, threonine kinase that is a key intracellular signaling node downstream of the mydossome associated Toll-Like (TLR 1, 2, 4, 5, 6, 7, 8, 9 and 10) and the interleukin (IL)-1 family receptors (IL-1R, IL-18R and IL-33R). There is emerging evidence of increased activation of these pathways in patients who succumb to SARS-COVID-19 compared to survivors. Rare genetic variants in humans lacking IRAK4 or MYD88 are susceptible to infections with pyogenic bacteria, but there is no evidence of increased viral susceptibility, presumably due to redundant innate pathways recognizing viral nucleic acids upstream of NF-κB. Furthermore, kinase inhibition preserves the IRAK4 scaffolding function within the myddosome and NF-κB activation downstream of TLR activation, suggesting a less immunosuppressive profile than the genetic deletion. In addition to inflammatory signals coming directly from the virus and anti-viral host response, a third driver of inflammatory tone is the massive production of cellular debris presenting damage associated molecular patterns (DAMPs) that also signal through the TLR family of receptors.

PF-06650833 is an investigational, selective and reversible inhibitor of IRAK4 that has demonstrated efficacy in a 12-week study in patients with moderately to severely active RA. Importantly, data generated in clinical studies in healthy volunteers and RA patients demonstrate that IRAK4 dose-dependently inhibits blood transcriptional programs for type I interferons and IL-6, and its most prominent cellular effect is a decrease in inflammatory monocytes. IRAK4 inhibitors also reduce chemokine production from human bronchial epithelial cells, specifically IL-1 β-stimulated release of CXCL8 and IL-6.

Inhibition of IL-1R and TLR family signaling by PF-06650833 will reduce excessive inflammatory responses to SARS-CoV-2 and mitigate lung injury leading to ARDS.

PF-06650833 has been generally well-tolerated with an acceptable safety profile based on clinical studies conducted to date in healthy participants and patients with RA. An exploratory exposure-response model suggests that maintaining PF-06650833 minimum observed concentration (Cmin) above the in vitro 90% inhibitory concentration ([IC90]) is desirable for maximal CRP reduction. A dose of 400 mg PF-06650833 MR tablets administered once QD is expected to inhibit TLR7/8 ligand-induced IL-6 production by approximately 90 and 95% at Cmin and Cmax, respectively, which would translate to about 50-60% reduction in CRP. Given the high mortality in COVID-19 complicated by ARDS, and the overall case mortality rate in in COVID-19 patients, a PF-06650833 dose of 400 mg MR tablets QD, fasted, is expected to provide maximal cytokine suppression while maintaining an overall positive benefit-risk profile.

The study will be a randomized, single-center, open-label, contemporary case-controlled, parallel group Phase 2 study of the efficacy and safety of PF-06650833 in hospitalized adult male and female patients with COVID-19 and pneumonia, who do not need mechanical ventilation to maintain adequate oxygenation. Up to approximately 57 patients to receive active treatment or standard-of-care (SoC) treatment only in a 2:1 ratio.

atients with confirmed SARS-CoV-2 infection, and meeting all other inclusion and exclusion criteria, who are randomized to active treatment will receive PF-06650833 400 mg modified release (MR) tablets once daily (QD) administered orally, fasted, for up to 28 days, or until discharge from the hospital or admission to the intensive care unit (ICU). All dosing of study drug will be in addition to current standard of care treatment, that must include some treatment targeting SARS-CoV-2 (eg, anti-malarial drugs such as chloroquine / hydroxychloroquine, and / or anti-viral therapies, some of which may also be experimental). The comparator group will consist of patients who will only receive standard of care treatment. Patients will be assessed daily while hospitalized for a variety of clinical, biomarker, safety, PK (as available), and laboratory parameters.

The inclusion criteria includes adult male and female patients, including women of childbearing potential, between 18 and 70 years of age, inclusive, capable of giving signed informed consent. Patients will have laboratory-confirmed novel coronavirus (SARS-CoV-2) infection and evidence of pneumonia assessed by all of the following: radiographic imaging (chest x-ray, chest CT scan, etc.); clinical assessment (evidence of rales/crackles on exam); and SpO2 ≤ 94% on room air. Patients will also have increased inflammation as assessed by at least one of the following being greater than the upper limit of normal (as available): hsCRP; ferritin; procalcitonin; D-dimers; fibrinogen; LDH; and PT/PTT.

The exclusion criteria will include another medical condition other than COVID-19 or laboratory abnormality that may increase the risk of study participation or, in the investigator’s judgment, make the participant inappropriate for the study, such as: acute coronary syndrome; chronic infections such as TB and HIV; requirement for mechanical ventilation, extracorporeal membrane oxygenation, or high flow ventilatory devices for respiratory support; PaO₂ / FiO₂ ratio < 300; proven bacterial pneumonia, other serious infection, sepsis, and / or septic shock; corticosteroids equivalent to prednisone or methylprednisolone > 20 mg/day; alanine transaminase/aspartate transaminase (ALT/AST) > 5 times the upper limit of normal; platelet count < 50,000/mm³; absolute neutrophil count < 1500/mm3; estimated glomerular filtration rate (eGFR) < 60 mL/min/1.73 m²); pregnancy; immunocompromised patients, patients with known immunodeficiencies or taking other immunosuppressives; and anticipated survival < 72 hours as assessed by the Investigator.

All standard of care treatments for hospitalized patients with COVID-19 are permitted during the period of treatment with study intervention. All patients should be receiving some treatment targeting SARS-COV-2. This includes both approved and experimental anti-viral treatments, and unapproved used of marketed drugs. To the extent possible, however, the following restrictions should be followed: acetaminophen / paracetamol dose should be limited to ≤ 2.6 g/day; corticosteroids should be limited to 20 mg prednisone/methylprednisolone/day; use of strong inhibitors/inducers of cytochrome P450 (CYP) 3A4 should be avoided to the extent possible. Judicious use is of strong inhibitors of CYP3A4 (eg, anti-viral treatment with ritonavir) is permitted, if necessary, with careful monitoring for adverse effects. All non-pharmacologic and nutritional supportive care measures, including nutritional supplementation and oxygenation support are permitted. All concomitant medications and nondrug treatments, with dose and frequency, should be recorded. Hormonal contraceptives that meet the requirements of this study are allowed to be used in participants who are WOCBP.

Discontinuation criteria includes: institution of mechanical ventilation or ECMO; discharge from the hospital; and SAEs (other than death), including serious infections other than SARS-CoV-2.

The primary objective will be to assess the efficacy of PF-06650833 in treating patients with COVID-19 pneumonia requiring hospital admission and evidence of elevated inflammatory state at time of presentation.

The secondary objectives will be to assess the effects of PF-06650833 on inflammatory biomarkers in patients with COVID-19 pneumonia and to assess the safety of PF-06650833 in patients with COVID-19 pneumonia.

The primary endpoints will include the percentage of patients in each of an ordinal scale of disease severity at the end of treatment (Day 29). The ordinal scale is an assessment of the clinical status at the first assessment of a given study day. The scale is as follows: 1) not hospitalized; 2) hospitalized, not requiring supplemental oxygen; 3) hospitalized, requiring supplemental oxygen; 4) hospitalized, on non-invasive ventilation or high flow oxygen devices; 5) hospitalized, on invasive mechanical ventilation or extracorporeal membrane oxygenation (ECMO); 6) death.

The secondary endpoints will include: the percentage of patients in each of an ordinal scale of disease severity with time (Days 1 - 28); time to clinical improvement (defined as a two-point increase on the ordinal scale); mortality rate; cure rate; proportion of patients requiring ICU admission and mechanical ventilatory support; change in National Early Warning Score (NEWS) from baseline (This score is based on 7 clinical parameters (respiration rate, oxygen saturation, any supplemental oxygen, temperature, systolic blood pressure, heart rate, level of consciousness); change from baseline in inflammatory parameters (e.g., hsCRP, procalcitonin, ferritin, D-dimers, LDH, fibrinogen, PT/PTT); change from baseline in cytokine panel (IL-1, IL-2, IL-6, IL-8, TNF-α, IL-17A, IL-17F, IP-10, CCL5), as available; safety as assessed by reporting of adverse events, changes in clinical laboratory parameters (e.g., hemoglobin, hepatic transaminases, serum creatinine, bilirubin).

### Example 5

### Investigation of IRAK4 Inhibition to Mitigate the Impact of COVID-19 in Severe SARS-CoV-2

Coronavirus disease-2019 (COVID-19) is a viral disease caused by a novel coronavirus, SARS-CoV-2, that has seen pandemic spread since its identification in January 2020 and that can cause a severe acute respiratory syndrome. While a majority of patients with COVID-19 may experience a self-limited, asymptomatic or mild illness, based on data from the Chinese experience in Wuhan, many patients may rapidly (within 1-2 weeks of infection) develop dyspnea and pneumonia and require hospitalization for respiratory support. Of these hospitalized patients, 20-30% have required admission to intensive care units (ICUs) for ventilatory support due to development of acute respiratory distress syndrome (ARDS), with ventilatory failure being a major cause of overall mortality due to COVID-19. Progression from milder respiratory involvement to ARDS is currently believed to be driven in large part to exuberant inflammation due to a cytokine release syndrome (CRS). There are currently no treatments for COVID-19 nor vaccines to prevent its further dissemination. There is therefore a need for therapeutic interventions that prevent the progression of infection to severe disease and death.

Patients with severe SARS-CoV-2 manifest cytokine release syndrome (CRS), associated with systemic inflammation, hemodynamic instability, multiple organ failure, and SARS-CoV-2 related mortality.

Multiple cytokines, including IL-6, are involved in CRS. Indeed, the IL-6 receptor antibody tocilizumab is FDA approved for the treatment of Chimeric Antigen Receptor T (CART) Cell-Induced Severe or Life-Threatening CRS. Anti-IL-6 antibody treatment is being used in some cases of SARS-CoV-2, and treatment with tocilizumab is standard of care as of Apr. 4, 2020. However, the only data to support the use of tocilizumab is a 21-patient retrospective case series from China. A therapy targeting multiple cytokines, including IL-6, may prove effective in SARS-CoV-2-induced CRS.

The exudative phase of CRS is thought to be due to an influx of neutrophils and macrophages and elevations of inflammatory cytokines, with higher levels of not only IL-6, but also IL-1, IL-8, and IL-18. SARS-CoV-nfected airway epithelial cells (AECs) produce large amounts of CCL3, CCL5, CCL2, and CXCL10. High serum levels of pro-inflammatory cytokines (IFN-y, IL-1, IL-6, IL-12, and TGFβ) and chemokines (CCL2, CXCL10, CXCL9, and IL-8) were also observed in SARS patients with severe disease compared to individuals with uncomplicated SARS. In addition to pro-inflammatory cytokines and chemokines, individuals with lethal SARS showed elevated levels of IFN (IFN-α and IFN-_{Y}) and IFN-stimulated genes (ISGs) (CXCL10 and CCL-2) compared to healthy controls or individuals with mild-moderate disease. Collectively, available data suggest that innate immunity pro-inflammatory signaling may be important in CRS and SARS that might be amenable to treatments targeting these pathways.

IRAK4 is a serine, threonine kinase that is a key intracellular signaling node downstream of the myddosome-associated Toll-Like Receptors (TLR) 1, 2, 4, 5, 6, 7, 8, 9 and 10, and the interleukin (IL)-1 family receptors (IL-1R, IL-18R and IL-33R) that mediate much of the human innate immune responses. In addition to inflammatory signals coming directly from the virus and anti-viral host response, the massive production of cellular debris that occurs in SARS present damage-associated molecular patterns (DAMPs) that also stimulate the TLR family of receptors. Thus, IRAK4 is an attractive target as a potential treatment for CRS due to SARS-CoV- infection.

Recent in vitro and in vivo experiments have implied that TLRs play a role in SARS infection and inflammation. It has been shown that pathogenic human coronaviruses induce oxidized phospholipids (OxPL) that promote acute lung injury by increasing lung macrophage cytokine/chemokine production via TLR4. Additionally, the SARS spike protein (S) has been shown to drive high levels of inflammatory cytokines through TLR2; and both TLR4 and TLR9 are induced by SARS-Cov infection and correlate with production of pro-inflammatory cytokines. Finally, SARS-CoV specific GU rich ssRNA fragments induce a high level of TNF-α, IL-6, and IL-12 via TLR7 and TLR8, suggesting that SARS-CoV GU-rich ssRNA may play a role in inducing inflammatory cytokines in SARS infection and ARDS

Inhibition of IRAK4 kinase activity blocks the production of cytokines such as type I interferons (IFN), the inflammatory cytokines IL-6, TNF-α, and IL-1β, as well as additional Th1 and Th17-inducing cytokines such as IL-12 and IL-23 that are key drivers of autoimmune and inflammatory diseases.

PF-06650833 suppressed inflammation in a number of preclinical models In an acute model of endotoxin-induced inflammation, PF-06650833 administered orally was found to inhibit LPS-induced TNFα in a dose-dependent manner in rats. Additionally, PF-06650833 was found to inhibit ear swelling in a dose-dependent manner in a 5-day murine model of imiquimod (a TLR7 agonist)-induced skin inflammation. In the rat collagen-induced arthritis model, PF-06650833 significantly reduced hind paw swelling. These data demonstrate that PF-06650833 is an effective inhibitor of TLR-induced inflammation in vivo.

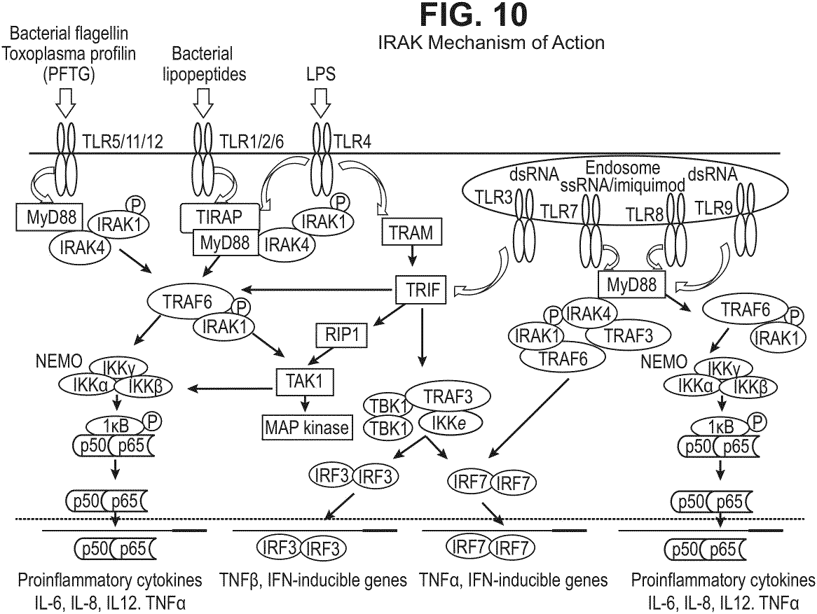

PF-06650833 dose-dependently reduced levels of hsCRP in healthy adults (FIG. 10).

PF-06650833 has been generally well-tolerated with an acceptable safety profile based on clinical studies conducted to date in healthy adults and patients with RA. An exploratory exposure-response model suggests that maintaining PF-06650833 minimum observed concentration (Cₘᵢₙ) above the in vitro 90% inhibitory concentration ([IC₉₀]) is desirable for maximal CRP reduction. A dose of 200 mg PF-06650833 IR suspension administered Q6H is expected to inhibit TLR7/8 ligand-induced IL-6 production by more than 90%, which would translate to about 50-60% reduction in CRP. Given the high mortality in COVID-19 complicated by ARDS, and the overall case mortality rate in COVID-19 patients, a PF-06650833 dose of 200 mg IR formulation Q6H is expected to provide maximal cytokine suppression while maintaining an overall positive benefit-risk profile.

If concomitant administration of a strong inhibitor of CYP3A (eg, ritonavir) cannot be avoided, a dose reduction of PF-06650833 to 200 mg IR QD (for subjects unable to take tablets orally) or 200 mg MR tablet (for subjects able to take tablets orally) is recommended and will provide high target coverage.

The effect of renal impairment on PK of PF-06650833 is not known. Given the low renal excretion of PF-06650833, it is expected to be safe without any dose reduction. However, effect on CV and laboratory parameters should be monitored. PF-06650833 should not be used with a strong CYP3A inhibitor in moderate to severely renally impaired patients

pplicant proposes a randomized, open-label, contemporary case-controlled, parallel group Phase 2 study of the efficacy and safety of PF-06650833, an investigational drug, in hospitalized adult male and female patients with SARS-CoV-2-induced acute respiratory distress syndrome (ARDS) who need mechanical ventilation.

Subjects will be screened within 24 hours of initiation of mechanical ventilation. Subjects with confirmed SARS-CoV-2 infection, and meeting all other inclusion and exclusion criteria, will be randomized within 48 hours of initiation of mechanical ventilation. Subjects who are randomized to active treatment will receive PF-06650833 200 mg immediate release (IR) suspension administered orally (via NG or OG tube) every 6 hours, starting at the time of study enrollment for up to 28 days or to an earlier occurrence of one of the following: death, discharge from the hospital, or 14 days after return to their clinical baseline (as defined by need for supplementary oxygen) prior to SARS-CoV-2. Subjects who are able to take tablets by mouth (PO) will receive 400 mg PF-06650833 as modified release (MR) tablets orally once daily (QD), preferably under fasted conditions (at least 4 hours after and 1.5 hours before a meal). All dosing of study drug will be in addition to current SOC treatment, which may include treatments targeting SARS-CoV-2 (see Concomitant Medications, Section 6.1.7)). Subjects will be assessed daily while hospitalized for a variety of clinical, biomarker, safety, and laboratory parameters.

The study population will be derived from the Intensive Care Unit and will consist of adult male and female subjects, aged 18 -75, inclusive, with laboratory-confirmed SARS-CoV-2 infection, evidence of increased inflammation, and severe acute respiratory syndrome requiring mechanical ventilation or extracorporeal membrane oxygenation. Up to a total of approximately 61 patients will be recruited to receive PF-06650833 in addition to standard of care or standard of care (SOC) treatment alone in a 2:1 ratio.

The primary objective of this Phase 2 study is to assess the efficacy of PF-06650833 in addition to standard-of-care compared to standard-of-care treatment alone in improving outcomes in patients with COVID-19, evidence of increased inflammation, and ARDS requiring mechanical ventilation or extracorporeal membrane oxygenation at time of admission.

The secondary objectives of this study are to determine whether treatment with PF-06650833 in addition to standard-of-care reduces mortality, improves clinical status, decreases hospital and ICU duration, and/or otherwise improves clinical status in severe Covid-19 compared to standard-of-care and to assess the safety of PF-06650833 in patients with severe Covid-19.

Subjects can expect to be in the study for up to 30 days, which will include an up to 2-day period before randomization, and up to 28 days of study drug administration. A follow-up assessment (which may be by phone) may be conducted up to 60 days after randomization to check on subject status.

### Example 6

### Tofacitinib Treatment for Patients With Early Onset SARS-CoV-2 Interstitial Pneumonia Multifocal Interstitial Pneumonia Represents the Most Common Cause of Admission in Intensive Care Units and Death in SARS-CoV-2 Infections, up to 25% of Admitted Patients with Pneumonitis Require Mechanical Ventilation or Oro-Tracheal Intubation within 5-10 Days

Although information about pathological and pathophysiological features of alveolar-interstitial damage is very limited, few available data, mostly collected on other coronavirus infections with similar clinical behaviour (SARS1 , MERS), seem to indicate as primary pathogenic mechanism an intense “cytokine storm” with a consequent inflammatory infiltrate of pulmonary interstitium, macrophage activation, giant cells formation and subsequent extended alveolar damage.

At present, no effective antiviral treatment is available. The most promising antiviral drug, Remdesivir, because of toxicity and limited availability, at least in Italy, is used only in severe refractory cases as rescue and/or compassionate therapy.

Preliminary evidence, however, is accumulating about the efficacy of an aggressive treatment of the corona virus-induced inflammation. Some case series have shown effectiveness of anti-IL6 strategies in reducing the severity of multifocal interstitial pneumonia in patients affected by SARS-CoV-2, implying a major role of IL-6 in the pathogenesis of lung damage in these patients and in clinical study recently approved by the italian regulatory agency (AIFA), Tocilizumab, which targets IL-6 receptor, is used without concomitant anti-viral therapy.

urthermore, intracellular signaling following IL-6 binding to the receptor (IL-6R) occurs mainly via JAK1, which is constitutively bound to the cytoplasmic part of gp130 and activated by gp130 dimerization. The generation of knockout mice confirmed that JAK1 is the dominant kinase activated by IL-6 in vivo and gp130 acts as a signaling receptor for additional cytokines such as IL-11, oncostatin M, ciliary neurotrophic factor, leukemia inhibitory factor, cardiotrophin-like cytokine factor which are considered part of the IL-6 family of cytokines. In-vitro studies show that Tofacitinib, both at 5 and 10 mg, inhibits IL-6/STAT3 signaling.

Thus, based on the above evidence, blocking JAK1 may be clinically rewarding in down-regulating IL-6 driven inflammation in patients with corona-virus infection.

Tofacitinib is a Janus kinase (JAK) inhibitor. JAKs are intracellular enzymes which transmit signals arising from cytokine or growth factor-receptor interactions on the cellular membrane to influence cellular processes of hematopoiesis and immune cell function. Within the signaling pathway, JAKs phosphorylate and activate Signal Transducers and Activators of Transcription (STATs) which modulate intracellular activity including gene expression. Tofacitinib modulates the signaling pathway at the point of JAKs, preventing the phosphorylation and activation of STATs. JAK enzymes transmit cytokine signaling through pairing of JAKs (e.g., JAK1/JAK3, JAK1/JAK2, JAK1/TyK2, JAK2/JAK2). Tofacitinib inhibited the in vitro activities of JAK1/JAK2, JAK1/JAK3, and JAK2/JAK2 combinations with IC₅₀ of 406, 56, and 1377 nM, respectively. Pharmacodynamic data showed that the maximum inhibition of anti-IL6 signaling requires 10 mg bid, at least in normal healthy individuals.

The safety profile of tofacitinib 10 mg BID is well characterized in RA, PsA, and UC patients for a duration of 8 weeks or longer. This dose is currently approved in the US and EU for induction therapy up to 16 weeks in UC patients with potential for longer term use if needed for maintenance of treatment benefit. Therefore, there are minimal safety concerns for short duration treatment with tofacitinib 10 mg BID

The primary objective of the study is to reduce the number of SARS-CoV-2 infected patients who need mechanical ventilation, and/or oro-tracheal intubation, to maintain PaO2/FiO2 greater than150 or, if PaO2 data not available, to maintain SO2 greater than 94% with FiO2 0.5.

The secondary objectives in SARS-CoV-2 infected patients are to prevent: virus-induced cytokine storm; development of severe pulmonary function deterioration; multiple organ dysfunction; and death.

Applicant proposes a prospective, singly cohort, open label Phase 2 clinical study. Patients will be evaluated at baseline (time 0) and followed for 14 days or until discharge. At baseline and every 24 h the following items will be assessed: hemodynamic and respiratory parameters; hemoglobin level; neutrophil and platelet counts; ALT; arterial blood test; glycaemia; pro-calcitonin; and CRP. At baseline and at day +7 and +14, 7 cc of serum will be stored to evaluate the serum levels of: IL-6; surfactant protein D; KL-6; and vWF levels. Patients who will need mechanical ventilation or ICU transfer within 24 hours from Hospital admission will be excluded from analysis. After 14 days from stopping the drug all patients will be evaluated through direct clinical examination or phone calling (if discharged). Any significant clinical event will be recorded in the CRF.

Patients infected with SARS-CoV-2 will receive immediate release (IR) Tofacitinib 10 mgs twice a day (BID) per os. Treatment will be started within 12 hours from admission and maintained for 14 days. The total daily dose will be reduced by 50% to 5 mgs twice a day, in: patients with severe renal impairment (GFR 30-60 ml/min); patients with moderate hepatic impairment (tofacitinib is not recommended in severe hepatic impairment); patients receiving potent inhibitors of CYP3A4 (e.g., itraconazole); and patients receiving one or more concomitant medications that result in both moderate inhibition of CYP3A4 and potent inhibition of CYP2C19 (e.g., fluconazole).

At the 10 mg BID dose, approximately 80% suppression of IL-6 may be expected, in addition to substantial inhibition of multiple other cytokines such as IFNγ, IL-15, IL-21, and IL-27.

t the 10 mg BID dose, plasma tofacitinib concentrations are maintained above the IC₅₀ for IL-6 inhibition throughout a 24-hour dosing interval at steady-state, unlike the lower dose of 5 mg BID. The 10 mg BID dose provides bettter anti-inflammatory effects, than 5 mg BID, for controlling potential increase in cytokines and progression to ARDS. The expected magnitude of cytokine suppression is lower at the 5 mg BID dose, with approximately 60% predicted suppression of IL-6.

Background treatment with an antiviral drug is required during treatment with IR tofacitinib 10 mg BID in COVID-19 infected patients. Some antiviral drugs have potential of drug-drug interactions (DDIs) and adjustment of tofacitinib dose may be required. Examples of antiviral drugs that have been evaluated, or are being used, for treatment of COVID-19 patients, and guidance for use of IR tofacitinib 10 mg BID with each drug, is provided below.

No DDI is expected between tofacitinib and hydroxychloroquine (HCQ) and dose adjustment of tofacitinib is not needed when dosed concomitantly with HCQ. Tofacitinib dose adjustment is not needed when the antibiotic azithromycin is co-administered, whether alone or in combination with HCQ.

While limited data is available for Remdesvir, this nucleoside analog antiviral drug is not expected to impact tofacitinib exposure and no dose adjustment for tofacitinib is recommended.

Available information for Favipiravir indicates that favipiravir is not a potent inhibitor or inducer of CYP3A4, and no dose adjustment of tofacitinib is recommended when co-administered.

Ritonavir and lopinavir are expected to increase tofacitinib exposure due to potent CYP3A4 inhibition. Therefore, when these drugs are co-administered with tofacitinib, individually or in combination, the tofacitinib dose should not exceed 5 mg BID

The available information for Oseltamivir indicates that oseltamivir is extensively metabolized by liver esterases and it is not known to cause potent inhibition or induction of CYP3A4. No dose adjustment of tofacitinib is recommended when co-administered.

No dose adjustment of tofacitinib is needed when co-administered with ribavirin.

All patients should be treated with hydroxychloroquine (400-600 mg/d) and Low Molecular Weight Heparin subcutaneously at prophylactic dosage.

In order to reduce risk for herpes zoster re-activation and bacterial superinfections, prophylaxis with acyclovir and co-trimoxazole will be started from day 1 to 14.

In patients who will need mechanical ventilation, Tofacitinib treatment will be stopped and rescue therapy started (e.g. Remdesavir and/or Tocilizumab), according to local protocols.

### Example 7

### A Multicenter Randomized, Double Blind, Placebo-Controlled Trial Was Performed Comparing Tofacitinib in Addition to the Standard of Care with Placebo and Standard of Care in Hospitalized Patients with Covid-19 Pneumonia

Patients aged 18 years or older with laboratory-confirmed SARS-CoV-2 infection as determined by polymerase-chain-reaction prior to randomization, evidence of Covid-19 pneumonia confirmed by radiographic imaging and who were hospitalized for less than 72 hours and receiving standard of care treatment according to local practice were included. Main exclusion criteria was the need for non-invasive ventilation, invasive mechanical ventilation, or extracorporeal membrane oxygenation (ECMO) on the day of randomization, prior history or current thrombosis, immunocompromised patients, and any current malignancy or lymphoproliferative disorders that required active treatment.

Eligible patients were randomized to receive tofacitinib or placebo in a 1:1 ratio in blocks of four, stratified according to site using a central concealed, web-based, automated randomization system. Patients assigned to tofacitinib received oral tofacitinib 10 mg twice daily for up to 14 days or until hospital discharge, whichever was earlier. A reduced dose regimen of 5 mg twice daily was given for patients with a calculated creatinine clearance (Cr Cl) <50 mL/min/1.73 m2, those with moderate hepatic impairment, and those with concomitant use of a strong CYP3A4 inhibitor, or a combination of a moderate CYP3A4 inhibitor and a strong CYP2C19 inhibitor. The standard of care treatment for Covid-19 at the time of the trial was administered at the discretion of the attending physician and could have included glucocorticoids, antibiotics, anticoagulants and/or antiviral agents.

Baseline assessments included demographics, relevant medical history, and laboratory data. Participants were assessed daily (up to day 28) while hospitalized. Follow-up visits occurred on day 14 and on day 28 for those who were discharged before day 14 or 28.

The primary outcome was the cumulative incidence of death or respiratory failure until day 28. This was defined when participants attained the categories 1, 2 or 3 on the 8-point National Institute of Allergy and Infectious Diseases (NIAID) ordinal scale of disease severity at any time during the 28 days of follow-up. For patients who were enrolled in the trial using high-flow oxygen devices (category 3), they were considered to have attained the primary outcome if they presented clinical worsening to categories 1 or 2. The occurrence of the primary outcome was adjudicated by an independent clinical events classification committee, whose members were unaware of the trial group assignments.

Secondary efficacy outcomes were the cumulative incidence of individual components of the primary outcome (all-cause mortality and respiratory failure), the occurrence of death or respiratory failure at day 28, the NIAID ordinal scale of disease severity at day 14, the NIAID ordinal scale of disease severity at day 28, the status of being alive and not using mechanical ventilation or ECMO at day 14 and day 28, the status of being alive and not hospitalized at day 14 and day 28, the status of being hospitalized and requiring supplemental oxygen at day 28, the cure defined as resolution of fever, cough, and need for ventilatory or oxygen support, the status of being in ICU or on ventilatory support at day 28, the duration of hospital stay, the duration of ICU stay, and the number of days free from mechanical ventilation at 28 days. The occurrence and severity of adverse events were evaluated. These events were coded according to the Medical Dictionary for Regulatory Activities (MedDRA) version 23.1.

The primary efficacy analyses included all randomized participants. The assignment of 260 patients with 1:1 randomization was estimated to provide at least 80% power to detect a between-group difference of 15 percentage points in the primary outcome, assuming an event rate (death or respiratory failure) of 15% in the tofacitinib group and 30% in the placebo group.

Baseline categorical variables are presented as relative and absolute frequencies. Baseline continuous variables are summarized using mean and standard deviation or median and interquartile range (IQR). Results for the primary outcome for efficacy (proportion of death or respiratory failure until day 28) were analyzed through binary regression with Firth correction, with the treatment and inclusion of antiviral therapy for Covid-19 as covariates. The risk ratio between groups and its 95% confidence intervals (CI) were calculated using a generalized linear model with binomial distribution adjusted to antiviral therapy. The antiviral treatments registered on day 1 were used in the statistical model. The effect of the intervention on in-hospital mortality until day 28 is expressed as hazard ratio (HR) derived from Cox regression. Dichotomous secondary outcomes were analyzed similarly to the primary outcome. For ordinal data, a proportional odds model adjusted for inclusion of antiviral therapy at baseline was used. An odds ratio (OR) of less than 1.0 represents a clinical improvement assessed on the ordinal scale. Safety analyzes included all randomized participants who took at least 1 dose of study intervention. A per protocol analysis was performed for the primary outcome excluding participants with major protocol deviations. Pre-specified subgroup analyses included age, sex, concomitant antiviral use, concomitant corticosteroid use, and time from symptom onset to randomization.

The 95% CIs are estimated for all effect measures. The widths of the CIs for the secondary outcomes were not adjusted for multiple comparisons, so the intervals should not be used to infer definitive treatment effects. All analyses were performed using SAS software version 9.4 and R software version 3.6.3 (R Foundation).

The primary outcome was initially the occurrence of death or respiratory failure at day 28. However, it was changed to the occurrence of death or respiratory failure until day 28 since the cumulative incidence of these events during the 28 days was deemed to be more clinically meaningful than the event rate at only one specific timepoint.

The baseline demographic and clinical characteristics were generally well balanced among the trial groups (Table 1).

TABLE 1

Characteristics of the patients at baseline*

| Characteristic | Tofacitinib (n=144) | Placebo (n=145) | Total (n=289) |
|---|---|---|---|
| Age, mean ± SD, y | 55.1 (14.4) | 57 (13.6) | 56 (14.0) |
| Distribution, no. (%) | | | |
| <45 years | 39 (27.1) | 30 (20.7) | 69 (23.9) |
| 45-64 years | 64 (44.4) | 67 (46.2) | 131 (45.3) |
| ≥65 years | 41 (28.5) | 48 (33.1) | 89 (30.8) |
| Female sex, no. (%) | 50 (34.7) | 51 (35.2) | 101 (34.9) |
| Race, no. (%)‡: | | | |
| White | 118 (81.9) | 123 (84.8) | 241 (83.4) |
| Black | 12 (8.3) | 13 (9.0) | 25 (8.7) |
| Mestizo | 11 (7.6) | 7 (4.8) | 18 (6.2) |
| Other or unknown | 3(2.1) | 2 (1.4) | 5 (1.7) |
| Body mass index, median (IQR), kg/m² | 29.4 (26.8-33.2) | 29.7 (26.4-32.7) | 29.7 (26.7-32.9) |
| Median time (IQR) from symptom onset to randomization - days | 10 (7-12) | 9 (7-11) | 10 (7-11) |
| Median time (IQR) from hospital admission to randomization - days | 5 (2-8) | 4 (2-8) | 5 (2-8) |
| Hospitalization at the ICU at randomization, no. (%) | 26 (18.1) | 24 (16.6) | 50 (17.3) |
| Medical history, no. (%) | | | |
| Hypertension | 67 (46.5) | 78 (53.8) | 145 (50.2) |
| Diabetes | 34 (23.6) | 34 (23.4) | 68 (23.5) |
| Dyslipidemia | 30 (20.8) | 20 (13.8) | 50 (17.3) |
| Current/ former tobacco use | 41 (28.5) | 41 (28.3) | 82 (28.4) |
| Score on ordinal scale, no. (%) | | | |
| 3. Hospitalized, requiring high-flow oxygen devices | 19 (13.2) | 18 (12.4) | 37 (12.8) |
| 4. Hospitalized, requiring supplemental oxygen | 91 (63.2) | 90 (62.1) | 181 (62.6) |
| 5. Hospitalized, not requiring supplemental oxygen, requiring ongoing medical care (Covid-19-related or otherwise) | 34 (23.6) | 37 (25.5) | 71 (24.6) |
| Treatments | | | |
| Corticosteroids | 114 (79.2) | 111 (76.6) | 225 (77.9) |
| Antivirals | 20 (13.9) | 18 (12.4) | 38 (13.1) |

ICU, intensive care unit; IQR, interquartile range; SD, standard deviation.
*The characteristics of the patients at baseline were well balanced among the trial groups.
‡Race group was determined by the investigator and recorded on the case-report form.

### Results

The Primary and Secondary Efficacy Outcomes are provided in Table 2.

TABLE 2

| Outcome | Tofacitinib (N = 144) | Placebo (N = 145) | |
|---|---|---|---|
| Primary Outcome | | | Risk ratio (95% Cl) |
| Death or respiratory failure until day 28 - no. (%) | 26 (18.1) | 42 (29.0) | 0.63 (0.41 to 0.97) |
| Secondary Outcomes | | | |
| | | | HR (95% Cl) |
| Mortality until day 28 - no. (%) | 4 (2.8) | 8 (5.5) | 0.49 (0.15 to 1.64) |
| | | | Risk ratio (95% Cl) |
| Respiratory failure until day 28 - no. (%) | 22 (15.3) | 34 (23.4) | 0.65 (0.40 to 1.05) |
| Death or respiratory failure at day 28 | 6 (4.2) | 12 (8.3) | 0.51 (0.20 to 1.31) |
| | | | OR (95% Cl) |
| NIAID ordinal scale at day 14 | | | 0.60 (0.36 to 1.00) |
| Distribution - no (%) | | | |
| 1 | 2 (1.4) | 5 (3.4) | |
| 2 | 7 (4.9) | 9 (6.2) | |
| 3 | 1 (0.7) | 6(4.1) | |
| 4 | 7 (4.9) | 6(4.1) | |
| 5 | 5 (3.5) | 6(4.1) | |
| 6 | 1 (0.7) | 2 (1.4) | |
| 7 | 11 (7.6) | 15 (10.3) | |
| 8 | 110 (76.4) | 96 (66.2) | |
| NIAID ordinal scale at day 28 | | | 0.54 (0.27 to 1.06) |
| Distribution - no (%) | | | |
| 1 | 4 (2.8) | 8 (5.5) | |
| 2 | 1 (0.7) | 4 (2.8) | |
| 3 | 1 (0.7) | 0 (0) | |
| 4 | 4 (2.8) | 1 (0.7) | |
| 5 | 0 (0) | 2 (1.4) | |
| 6 | 0 (0) | 1 (0.7) | |
| 7 | 5 (3.5) | 10 (6.9) | |
| 8 | 129 (89.6) | 119 (82.1) | |
| | | | Risk ratio (95% Cl) |
| Status of being alive and not using mechanical ventilation or ECMO (categories 3 to 8 of the ordinal scale) at day 14 | 135 (93.8) | 131 (90.3) | 1.57 (0.67 to 3.70) |
| Status of being alive and not hospitalized (categories 7 to 8 of the ordinal scale) at day 14 | 121 (84.0) | 111 (76.6) | 1.61 (0.90 to 2.90) |
| Status of being alive and not using mechanical ventilation or ECMO (categories 3 to 8 of the ordinal scale) at day 28 | 139 (96.5) | 133 (91.7) | 2.36 (0.85 to 6.57) |
| Status of being alive and not hospitalized (categories 7 to 8 of the ordinal scale) at day 28 | 134 (93.1) | 129 (89.0) | 1.63 (0.73 to 3.70) |
| Status of being hospitalized, requiring supplemental oxygen (categories 1 to 4 of the ordinal scale) at day 28 | 10 (6.9) | 13 (9.0) | 0.77 (0.33 to 1.77) |
| Cure* | 134 (93.1) | 132 (91.0) | 1.30 (0.56 to 3.01) |
| Status of being in the ICU or on ventilatory support at day 28 | 4 (2.9) | 5 (3.6) | 0.80 (0.23 to 2.76) |

*Cure refers to resolution of fever, cough, and need for ventilatory or oxygen support. CI denotes confidence interval; HR, hazard ratio; ICU, intensive care unit; IQR, interquartile range; NIAID National Institute of Allergy and Infectious Diseases.

1. A method of treating a patient infected with SARS-CoV-2 comprising administering to the patient in need of such treatment a therapeutically effective amount of tofacitinib or a pharmaceutically acceptable salt thereof.

2. The A method of claim 1 wherein the therapeutically effective amount of tofacitinib is administered as 5 mgs of tofacitinib BID or an equivalent amount of tofacitinib in the form of a pharmaceutically acceptable salt thereof.

3. The A method of claim 1 wherein the therapeutically effective amount of tofacitinib is administered as 10 mgs of tofacitinib BID or an equivalent amount of tofacitinib in the form of a pharmaceutically acceptable salt thereof.

4. The A method of claim 1 wherein the therapeutically effective amount of tofacitinib is administered as 11 mgs of tofacitinib QD or an equivalent amount of tofacitinib in the form of a pharmaceutically acceptable salt thereof.

5. The A method of claim 1 , wherein IL-6 levels are reduced by at least 20%.

6. The A method of claim 1 wherein the therapeutically effective amount of tofacitinib is administered orally in an amount from 2.5-15 mgs of tofacitinib, or an equivalent amount of tofacitinib in the form of a pharmaceutically acceptable salt thereof, wherein IL-6 levels are reduced by at least 20%.

7. The A method of claim 3 , wherein IL-6 levels are reduced by at least 20%.

8. The A method of claim 7 , wherein IL-6 levels are reduced by at least 50%.

9. The A method of claim 8 , wherein IL-6 levels are reduced by at least 75%.

10. The A method of claim 3 , wherein IL-6 and IL-8 levels are each reduced by at least 35%.

11. The A method of claim 3 , wherein IL-6, IL-8, and TNFα levels are each reduced by at least 35%.

12. The A method of claim 3 , wherein IL-6, IFNα, IFNβ, and TNFα levels are each reduced by at least 35%.

13. The A method of claim 3 , wherein IFNy, IL-6, IL-15, IL-21, and IL-27 levels are each reduced by at least 20%.

14. The A method of claim 3 , wherein IFNα, IFNγ, IL-2, IL4, IL-6, IL-7, IL-10, IL-12, IL13, IL-15, and IL-23 levels are each reduced by at least 20%.

15. A method of treating a patient infected with SARS-CoV-2 comprising administering to the patient in need of such treatment 400 mgs of 1-(((2S,3S,4S)-3-Ethyl-4-fluoro-5-oxopyrrolidin-2-yl)methoxy)-7-methoxvisoquinoline-6- carboxamide QD or an equivalent amount of 1-(((2S,3S,4S)-3-Ethyl-4-fluoro-5-oxopyrrolidin-2-yl)methoxy)-7-methoxyisoquinoline-6- carboxamide PF-06650833 in the form of a pharmaceutically acceptable salt thereof.

16. A method of treating a patient infected with SARS-CoV-2 comprising administering to the patient in need of such treatment 200 mgs of 1-(((2S,3S,4S)-3-Ethyl-4-fluoro-5-oxopyrrolidin-2-yl)methoxy)-7-methoxyisoquinoline-6- carboxamide Q6H or an equivalent amount of 1-(((2S,3S,4S)-3-Ethyl-4-fluoro-5-oxopyrrolidin-2-yl)methoxy)-7-methoxyisoquinoline-6- carboxamide in the form of a pharmaceutically acceptable salt thereof.

17. (canceled)

18. A method of treating a patient infected with SARS-CoV-2 comprising administering to the patient in need of such treatment 100-800 mgs of 1-(((2S,3S,4S)-3-Ethyl-4-fluoro-5-oxopyrrolidin-2-yl)methoxy)-7-methoxyisoquinoline-6-carboxamide , or an equivalent amount of 1-(((2S,3S,4S)-3-Ethyl-4-fluoro-5-oxopyrrolidin-2-yl)methoxy)-7-methoxyisoquinoline-6- carboxamide in the form of a pharmaceutically acceptable salt thereof, wherein IL-6 levels are reduced by at least 75%.

19. The A method of claim 15 , wherein IL-6 levels are reduced by at least 75%.

20. The A method of claim 16 , wherein IL-6 levels are reduced by at least 75%.

21. The A method of claim 15 , wherein IL-6 IL-1, and TNFα levels are each reduced by at least 20%.

22. The A method of claim 16 , wherein IL-6 IL-1, and TNFα levels are each reduced by at least 20%.

23. The A method of claim 15 , wherein IL-6 IL-1, TNFα, IFNα, and IFNγ levels are each reduced by at least 20%.

24. The A method of claim 16 , wherein IL-6 IL-1, TNFα, IFNα, and IFNγ levels are each reduced by at least 20%.

25. The A method of claim 15 , wherein IL-6 IL-1, TNFα, IFNα, IFNγ, and CXCL8 levels are each reduced by at least 20%.

26. The A method of claim 16 , wherein IL-6 IL-1, TNFα, IFNα, IFNγ, and CXCL8 levels are each reduced by at least 20%.

27. The A method of claim 15 , wherein CRP levels are reduced by at least 50% and wherein IL-6 IL-1, TNFα, IFNα, IFNγ, and CXCL8 levels are each reduced by at least 20%.

28. The A method of claim 16 , wherein CRP levels are reduced by at least 50% and wherein IL-6 IL-1, TNFα, IFNα, IFNγ, and CXCL8 levels are each reduced by at least 20%.
